(19) United States
(12) Patent Application Publication
(10) Pub. No.: US 2022/0288277 A1
(43) Pub. Date: Sep. 15, 2022

(54) SURFACE COATINGS AND IMPLANTABLE DEVICES COMPRISING DIMERIC STEROID PRODRUGS, AND USES THEREOF

(71) Applicant: Ripple Therapeutics Corporation, Toronto (CA)

(72) Inventors: Ian Charles PARRAG, Mississauga (CA); Matthew Alexander John STATHAM, Milton (CA); Dimitra LOUKA, Toronto (CA); Kyle Giovanni BATTISTON, Toronto (CA); J. Paul SANTERRE, Toronto (CA); Wendy Alison NAIMARK, Toronto (CA); Bernadette ILAGAN, Woodbridge (CA); Gillian Claire MACKEY, Toronto (CA); Hans Christian FISCHER, Toronto (CA); Jamie Robert SWENOR, Toronto (CA); Kelli-Anne Nicole WOOTTON, Toronto (CA)

(21) Appl. No.: 17/625,708

(22) PCT Filed: Jul. 9, 2020
(86) PCT No.: PCT/IB2020/000663

§ 371 Date: Jan. 7, 2022

Related U.S. Application Data
(60) Provisional application No. 62/880,737, filed on Jul. 31, 2019.
(60) Provisional application No. 62/872,506, filed on Jul. 10, 2019.

(51) Int. Cl. A61L 27/54; A61L 27/28

(52) U.S. Cl. CPC A61L 27/54; A61L 27/28; A61L 2300/222; A61L 2300/43; A61L 2400/18

(57) ABSTRACT

The disclosure features surface coatings formed from dimeric steroid prodrugs for the extended delivery of a drug from a surface, and for the treatment of a disease or condition. Also provided herein are drug depots formed from dimeric steroid prodrugs for the extended delivery of a drug for use in combination with implantable medical devices. Said dimeric steroid prodrugs are represented by the formula D1-L-D2, wherein D1 and D2 are independently a steroid radical and L is a linker covalently linking D1 to D2.

### CROSS-REFERENCE

This application claims the benefit of U.S. Provisional Application No. 62/872,506, filed Jul. 10, 2019, and U.S. Provisional Application No. 62/880,737, filed Jul. 31, 2019, which are each hereby incorporated by reference in their entirety herein.

### BACKGROUND OF THE DISCLOSURE

The appropriate biological response to the surface of a device is crucial for biocompatibility. A medical device, or the coating thereof can also serve as a repository for delivery of a biologically active agent (e.g., as with medical devices including drug depots). A coating or drug depot that is used to control release of the drug must avoid triggering an adverse biological response (e.g. not induce an inflammatory response), must produce the desired release profile, and must not adversely affect the mechanical or other critical properties required of the medical device. Further, when the active agent is a pharmaceutical drug, it is often desirable to release the drug locally from the medical device over an extended period of time.

There exists a need for drug delivery platforms which provide for delivery of biologically active agents with a defined profile of release.

### SUMMARY OF THE DISCLOSURE

Provided in certain embodiments herein are multi-component systems, such as comprising a first component and a second component. In some embodiments, the first component comprises an implantable article, such as an implantable sensor, an implantable drug depot or delivery device, or other implantable article or device, such as an implantable article or device that is observed to or is suspected may result in an adverse biological response (e.g., an inflammatory response). In certain embodiments, the second component is in proximal relation to the first component or is configured to be in proximal relation to the first component when the first component is implanted in an individual. In some embodiments, the second component is a coating configured on a surface of the first component (e.g., at least partially coating one or more surface of the first component). In certain embodiments, the second component is a second article (e.g., a co-implant) that is affixed or adhered to the first component (e.g., a surface thereof). In other embodiments, the second component is comprised of a distinct article that, when implanted into an individual, is implanted in such a manner as to be configured in proximal relation to the first component. In certain instances, when the second component is configured in a proximal relation to the first component, an adverse biological response (e.g., inflammatory response) is prevented or inhibited (e.g., reduced relative to a response that would be observed if the first component was administered in the absence of the second component). In certain embodiments, also provided herein, is the second component, independent of its relationship with the first component.

In certain embodiments, a second component described herein comprises any suitable compound provided herein, such as a compound of formula (A-VIII):

D1-L-D2 (A-VIII),

or a pharmaceutically acceptable salt thereof.

In certain embodiments, each of D1 and D2 is, independently, a radical formed from a steroid (e.g., also referred to herein as a steroid or a steroid radical). In certain embodiments, L is a linker joining D1 to D2. In specific embodiments, L is a linker that covalently joins D1 to D2.

In some embodiments, the second component comprises any suitable amount of the compound (e.g., of formula (A-VIII)). In some embodiments, the second component comprises at least 50% (w/w) (e.g., at least 60% (w/w), at least 70% (w/w), at least 80% (w/w), at least 90% (w/w), at least 95% (w/w), at least 98% (w/w), at least 99% (w/w), or the like). In some embodiments, the second component is free or substantially free of a controlled release excipient, such as a controlled release (e.g., polymer) matrix. In certain embodiments, (e.g., even without or with low concentrations of a controlled-release excipient) components (e.g., coatings or co-implants) comprising (e.g., high concentrations, such as described herein) of compound have good release profiles and/or kinetics. For example, in some instances, extended release of the compound and/or its component parts (e.g., D1 and D2 in their free (non-radical) forms) in tissue (e.g., of an individual), serum (e.g., of an individual or bovine serum (e.g., as a standard utilized to measure release kinetics and/or profile)), or a buffer (such as phosphate buffered saline, PBS). Such release profiles are determined at any suitable temperature, such as about 37° C. (e.g., in the body of an individual, or as a temperature to mimic the temperature of an individual). In some embodiments, extended release of the compound and/or its component parts (e.g., in free form) is achieved over a period of at least 1 month, at least 2 months, at least 3 months, or more (e.g., under the conditions described herein). In certain embodiments, release of the compound and/or component parts thereof is zero order or near zero order.

In a specific embodiment, provided herein is an article (e.g., an implantable device, such as a sensor) is coated with a second component, such as described herein. In other specific embodiments, provided herein is a first article (e.g., an implantable device, such as a sensor) with a second article affixed to the first article, such as wherein the second article is or comprises a second component, such as described herein. In still another embodiment, provided herein is a kit comprising a first article (e.g., an implantable device, such as a sensor) and a second article, such as comprising a second component, such as described herein. In certain embodiments, provided herein is a composition comprising a first article (e.g., an implantable device, such as a sensor) and a second article, such as comprising a second component, such as described herein. In some embodiments, provided herein is a method of implanting a first article (e.g., an implantable device, such as a sensor) in into an individual (e.g., in need thereof) comprising a first article; the process comprising (1) implanting the first article into the individual; and (2) implanting the second article (e.g., such as comprising a second component, such as described herein) into the individual, the second article being implanted in proximity to the first article. In specific embodiments, the second article is implanted close enough to the first article such as to prevent or inhibit an adverse biological response (e.g., inflammatory response) (e.g., such adverse biological response being reduced relative to a response that would be observed if the first component was implanted in the absence of the second component).

In certain embodiments, provided herein is a system comprising an article body and a steroid material. In some embodiments, provided herein is an article comprising an article body and a steroid material. In some embodiments, the steroid material comprising a compound of formula (A-VIII):

D1-L-D2 (A-VIII)

or a pharmaceutically acceptable salt thereof.

In some embodiments, each of D1 and D2 is, independently, a steroid radical; and L is a linker covalently linking D1 to D2. In some embodiments, the steroid material comprises the compound of formula (A-VIII) in an amount of at least 90% (w/w).

In some embodiments, provided herein is a system comprising an article body and a steroid material, the steroid material comprising a compound of formula (A-VIII):

D1-L-D2 (A-VIII)

or a pharmaceutically acceptable salt thereof,

wherein

(i) each of D1 and D2 is, independently, a steroid radical; and L is a linker covalently linking D1 to D2, and

(ii) the steroid material comprises the compound of formula (A-VIII) in an amount of at least 90% (w/w).

In some embodiments, provided herein is an article comprising an article body and a steroid material, the steroid material comprising a compound of formula (A-VIII):

D1-L-D2 (A-VIII)

or a pharmaceutically acceptable salt thereof,

wherein

(i) each of D1 and D2 is, independently, a steroid radical; and L is a linker covalently linking D1 to D2, and

(ii) the steroid material comprises the compound of formula (A-VIII) in an amount of at least 90% (w/w).

In some embodiments, the steroid material is in the form of a second body. In some embodiments, the second body is packaged with the article body. In some embodiments, the second body is packaged as a kit with the article body. In some embodiments, the second body is separate from the article body. In some embodiments, the second body is affixed to the article body. In some embodiments, the second body is affixed directly to the article body. In some embodiments, the second body is affixed to the article body with an adhesive, a clamp, a washer, a bolt, or the like (e.g., a screw).

In some embodiments, the steroid material is a surface coating, a drug depot, article, or other material or form described herein such as comprising a compound described herein.

In some embodiments, the steroid material is in the form of a coating. In some embodiments, the steroid material is a coating on the article body. In some embodiments, the steroid material coats at least one surface of the article body. In some embodiments, the steroid material is a coating on at least partially covers the article body. In some embodiments, the steroid material is in the form of a coating, the coating being on (e.g., at least partially covering) at least one surface of the article body.

In some embodiments, the article body is an implant. In some embodiments, the article body is a sensor implant. In some embodiments, the article body is an implantable device. In some embodiments, the article body is an implantable medical device. In some embodiments, the article body is an implantable device provided herein, such as, for example, prostheses, a mesh, a stent, or the like.

In some embodiments, the steroid material comprises less than 5 wt. %, less than 2 wt. %, or less than 1 wt. % of a controlled release excipient. In some embodiments, the steroid material (e.g., the second body or the coating) is free of a controlled release excipient.

In some embodiments, the steroid material provides controlled release of free steroid therefrom. In some embodiments, the steroid material provides release of free steroid therefrom without the need of a controlled release excipient. In some embodiments, the steroid material (e.g., the article body or the coating) releases D1 and D2 at a controlled rate. In some embodiments, the steroid material (e.g., the article body or the coating) releases D1 and D2 at a steady rate. In some embodiments, the steroid material (e.g., the article body or the coating) releases D1 and D2 at a controlled and steady rate. In some embodiments, the steroid material (e.g., the article body or the coating) releases D1 and D2 through continuous dissolution of the outermost layer(s) of the steroid material. In some embodiments, the steroid material (e.g., the article body or the coating) releases D1 and D2 through surface erosion. In some embodiments, the steroid material (e.g., the article body or the coating) releases D1 and D2 at a dissolution rate of the steroid material (e.g., the article body or the coating) in bovine serum or phosphate buffered saline (PBS). In some embodiments, the steroid material (e.g., the article body or the coating) releases D1 and D2 at 37° C. in bovine serum or in phosphate buffered saline (PBS) at a rate such that t₁₀ is greater than or equal to 1/10 of t₅₀.

In some embodiments, the steroid material (e.g., the article body or the coating) comprises at least 0.01% (w/w), 0.1% (w/w), 1% (w/w), 10% (w/w), or more of one or more plasticizing agents. In some embodiments, the steroid material (e.g., the article body or the coating) comprises at most 10% (w/w), 1% (w/w), 0.1% (w/w), 0.01% (w/w), or less of one or more plasticizing agents. In some embodiments, the steroid material (e.g., the article body or the coating) comprises from 0.01 to 10% (w/w) of one or more plasticizing agents.

In some embodiments, the steroid material is a surface coating. In some embodiments, the coating coats the entire article body (e.g., a washer). In some embodiments, the surface coating coats a fraction of the article body. In some embodiments, the surface coating coats at most half, one-quarter, one-eighth, one-sixteenth, or less of the article body. In some embodiments, the surface coating coats at least one-sixteenth, one-eighth, one-fourth, half, or more of the article body.

In some embodiments, the surface coating is a continuous layer on the article body. In some embodiments, the surface coating is a continuous layer on at least a portion of the article body. In some embodiments, the surface coating does not contain cracks, fissures, gaps, or the like. In some embodiments, the surface coating dissolves such that the layer of surface coating remains continuous.

In some embodiments, the steroid material is a co-implant. In some embodiments, the co-implant is a drug depot. In some embodiments, the steroid material is a drug depot co-implanted with the article body.

In some embodiments, the co-implant (e.g., the drug depot co-implanted with the article body) is separate from the article body. In some embodiments, the co-implant is implanted separate from the article body.

In some embodiments, the co-implant (e.g., the drug depot co-implanted with the article body) is selected from a pellet, a cylinder, a hollow tube, a microparticle, a nanoparticle, or a shaped article.

In some embodiments, the co-implant is implanted on or with the article body.

In some embodiments, the article body is an implantable medical device. In some embodiments, the co-implant (e.g., the drug depot co-implanted with the article body) is affixed to the article body. In some embodiments, the co-implant is adhesively affixed, screwed, bolted, or otherwise attached to the article body.

In some embodiments, the co-implant is coated with the steroid material. In some embodiments, the co-implant is a coated device or a coated article. In some embodiments, the co-implant is coated medical hardware. In some embodiments, the coated medical hardware is a component of a device. In some embodiments, the device is a medical device. In some embodiments, the device is an implantable medical device.

In some embodiments, D1 and D2 are each a steroid provided herein, or pharmaceutically acceptable salts thereof, in their free form. In some embodiments, D1 and D2 are each an anti-inflammatory steroid, or pharmaceutically acceptable salts thereof, in their free form. In some embodiments, D1 and D2 are each an anti-inflammatory steroid provided herein, or pharmaceutically acceptable salts thereof, in their free form. In some embodiments, D1 and D2 are each a corticosteroid (e.g., as provided herein), or pharmaceutically acceptable salts thereof, in their free form. In some embodiments, D1 and D2 are each a glucocorticoid (e.g., as provided herein), or pharmaceutically acceptable salts thereof, in their free form. In some embodiments, D1 and D2 are each independently selected from dexamethasone, triamcinolone, triamcinolone acetonide, prednisolone, hydrocortisone, betamethasone, and prednisone, pharmaceutically acceptable salts thereof, in their free form. In some embodiments, D1 and D2 are each dexamethasone, or pharmaceutically acceptable salts thereof, in their free form. In some embodiments, D1 and D2 are each intraocular pressure (IOP) lowering steroids, or pharmaceutically acceptable salts thereof, in their free form. In some embodiments, D1 and D2 are each intraocular pressure (IOP) lowering steroids provided herein, or pharmaceutically acceptable salts thereof, in their free form. In some embodiments, D1 and D2 are each anecortave, or pharmaceutically acceptable salts thereof, in their free form.

Provided in some embodiments herein is a method of providing an implant into an individual. In some embodiments, the method comprises implanting an implant article into the individual at an implant location. In some embodiments, the method comprises implanting a steroid material into the individual. In some embodiments, the steroid material is implanted in proximity to the implant location. In some embodiments, the steroid material is implanted within 20 mm, within 10 mm, within 5 mm, within 3 mm, or less of the implant location. In some embodiments, the steroid material is implanted within 3 mm, within 5 mm, within 10 mm, within 20 mm, or more of the implant location. In some embodiments, the steroid material is implanted within 3 mm to 20 mm of the implant location. In some embodiments, the steroid material is as described elsewhere herein.

In certain embodiments, provided herein is a method of providing an implant into an individual, the method comprising (i) implanting an implant article into the individual at an implant location, and (ii) implanting a steroid material into the individual, the steroid material being implanted in proximity (e.g., within 20 mm, within 10 mm, within 5 mm, within 3 mm, or less) to the implant location, the steroid material is as described elsewhere herein.

In some embodiments, the implant article and the steroid material are administered concurrently. In some embodiments, the steroid material is affixed to or coated on the implant article. In some embodiments, the implant article and the steroid material are administered concurrently, and the steroid material is affixed to the implant article. In some embodiments, the implant article and the steroid material are administered concurrently, and the steroid material is coated on the implant article. In some embodiments, the implant article and/or the steroid material are administered post-surgery.

In some embodiments, the implant article and the steroid material are administered sequentially. In some embodiments, the implant article is implanted before the steroid material. In some embodiments, the implant article is implanted after the steroid material. In some embodiments, the implant article and/or the steroid material are administered post-surgery.

In some embodiments, the steroid material remains implanted in the individual for the duration of a post-operative procedure. In some embodiments, the steroid material remains implanted in the individual for the duration of the life-span of the article body. In some embodiments, the steroid material remains implanted in the individual for the duration of the recovery period of the individual. In some embodiments, the steroid material remains implanted in the individual for at least 1 day, 1 week, 2 weeks, 1 month, or longer. In some embodiments, the steroid material remains implanted in the individual for at most 1 month, 2 weeks, 1 week, 1 day, or less.

In some embodiments, the steroid material, or a release product thereof, is uptaken by the individual for the duration of the recovery period of the individual. In some embodiments, at least a portion of the steroid material, or a release product thereof, is uptaken by the individual at a rate sufficient to produce a physiological effect in the individual. In some embodiments, at least a portion of the steroid material is uptaken by the individual at a rate sufficient to produce a physiological effect in or around the implant location of the individual. In some embodiments, the rate at 37° C. in 100% bovine serum or at 37° C. in PBS is such that t₁₀ is greater than or equal to 1/10 of t₅₀.

In some embodiments, the steroid material, or a release product thereof, reduces inflammation, reduces pressure, or the like in or around the implant location. In some embodiments, the steroid material, or a release product thereof, reduces an inflammatory response, lower intraocular pressure (IOP), or the like in or around the implant location. In some embodiments, the implant location is described elsewhere herein.

In some embodiments, inflammation in or around the implant location is reduced by at least 10%, by at least 20%, by at least 30%, by at least 50%. In some embodiments, pressure in or around the implant location is reduced by at least 10%, by at least 20%, by at least 30%, by at least 50%).

In some embodiments, inflammation in or around the implant location is measured by fibrotic layer thickness (e.g., μm), collagen content (e.g., μM), hydroxyproline content (e.g., μM), inflammatory cell count (e.g. number of macrophages, foreign body giant cells, etc.), inflammatory cell type (e.g. myofibroblasts), inflammatory cytokines (e.g. IL-1β, TNF-α, etc.), or the like.

In certain embodiments, the invention features an article including a surface coating, wherein the surface coating includes a compound of formula (A-VIII):

D1-L-D2 (A-VIII),

or a pharmaceutically acceptable salt thereof, wherein (i) each of D1 and D2 is, independently, a radical formed from a steroid; and L is a linker covalently linking D1 to D2, (ii) at least 90% (w/w) (e.g., at least 92%, 94%, 96%, 98%, 99%, or more (w/w)) of the surface coating is the compound of formula (A-VIII), (iii) the surface coating is free of controlled release excipient, and (iv) D1 and D2 is released from the coated surface at 37° C. in 100% bovine serum or at 37° C. in PBS at a rate such that t₁₀ is greater than or equal to 1/10 of t₅₀.

In some embodiments, the surface coating is a controlled release surface coating.

In certain instances, the invention features an implantable medical device including a drug depot, wherein the drug depot includes a compound of formula (A-VIII):

D1-L-D2 (A-VIII),

or a pharmaceutically acceptable salt thereof, wherein (i) each of D1 and D2 is, independently, a radical formed from a steroid; and L is a linker covalently linking D1 to D2, (ii) at least 70% (w/w) (e.g., at least 75%, 80%, 85%, 90% 92%, 94%, 96%, 98%, or 99% (w/w)) of the drug depot is the compound of formula (A-VIII), (iii) the drug depot is free of controlled release polymer, and (iv) D1 and D2 is released from the drug depot at 37° C. in 100% bovine serum or at 37° C. in PBS at a rate such that t₁₀ is greater than or equal to 1/10 of t₅₀.

In one embodiment, the implantable medical device includes a reservoir or holder for retaining the drug depot. The reservoir can include a drug depot in the form of a fiber, fiber mesh, woven fabric, non-woven fabric, pellet, cylinder, hollow tube, microparticle, nanoparticle, or shaped article. In particular embodiments, the holder includes a drug depot in the form of a sheath, collar, ring, washer, fibrous pouch, or threaded shaped article configured for placement within or upon the holder such that the drug depot is retained by the implantable medical device or becomes a direct component of the medical device. In other embodiments, the drug depot is adhesively affixed to said implantable medical device. The drug depot adhesively affixed to the implantable medical device can be in the form of a fiber, fiber mesh, woven fabric, non-woven fabric, wafer, sheet, film, pellet, cylinder, hollow tube, microparticle, nanoparticle, or shaped article. In other embodiments, the drug depot is not adhesively affixed and is held by the device through mechanical components (e.g. screw, nut, bolt, washers) or becomes a part of the device on its own (e.g. a fibrous pouch around the device).

In a related aspect, the invention features an article including a surface coating, wherein the surface coating includes a compound of formula (A-VIII):

D1-L-D2 (A-VIII),

or a pharmaceutically acceptable salt thereof, wherein (i) each of D1 and D2 is, independently, a radical formed from a steroid; and L is a linker covalently linking D1 to D2, (ii) at least 90% (w/w) (e.g., at least 92%, 94%, 96%, 98%, or 99% (w/w)) of the surface coating is the compound of formula (A-VIII), (iii) the surface coating includes from 0.01 to 10% (w/w) (e.g., from 0.1 to 1%, 0.5 to 2%, 1 to 5%, or 2 to 8% (w/w)) of one or more plasticizing agents, and (iv) the surface coating is free of controlled release excipient.

In particular embodiments, the one or more plasticizing agents are selected from glycerol, ethylene glycol, diethylene glycol, triethylene glycol, tetraethylene glycol, polyethylene glycol, propylene glycol, triacetin, sorbitol, mannitol, xylitol, fatty acids, monosaccharides (e.g., glucose, mannose, fructose, sucrose), ethanolamine, urea, triethanolamine, vegetable oils, lecithin, and waxes.

In certain embodiments, the invention features an article including a surface coating, wherein the surface coating includes a compound of formula (A-VIII):

D1-L-D2 (A-VIII),

or a pharmaceutically acceptable salt thereof, wherein (i) each of D1 and D2 is, independently, a radical formed from a steroid; and L is a linker covalently linking D1 to D2, (ii) at least 90% (w/w) (e.g., at least 92%, 94%, 96%, 98%, 99%, or more (w/w)) of the surface coating is the compound of formula (A-VIII), and wherein the surface coating is applied to a surface of the article by (a) dissolving the compound of formula (A-VIII) in a solvent to form a solution, (b) applying the solution to the surface of the article, (c) evaporating solvent on the surface of the article to form the surface coating, and (d) repeating (b) and (c).

In particular embodiments, the solvent is selected from tetrahydrofuran, N,N-dimethylformamide, diethylamine, chloroform, methyl t-butyl ether, toluene, benzene, ether, p-xylene, carbon disulfide, carbon tetrachloride, cyclohexane, pentane, hexane, heptane, dioxane, ethylacetate, dimethoxyethane, ethyl benzoate, anisol, chlorobenzene, pyridine, acetone, dimethylsulfoxide, acetonitrile, ethanol, n-propanol, toluene, methanol, benzyl alcohol, and mixture thereof. The concentration of the compound of formula (A-VIII) in the solution can be between 10 and 250 mg/mL (e.g., between 10 and 50, 25 and 75, 60 and 120, 80 and 150 mg/mL, or 125 and 250 mg/mL). In particular embodiments, (b) and (c) are repeated from 2 to 100 times (e.g., repeated 2 to 25, 10 to 50, 20 to 75, or 40 to 100 times). (b) can include dip coating, drop coating, drop and drag coating, or spray coating the solution onto the surface of the article or electrospinning or electrospraying the solution to form the surface coating. In certain embodiments, (c) includes evaporating the solvent to form the surface coating having a glassy state composition. In other embodiments, following (c) the surface coating is annealed. In some embodiments, the glassy state composition is an amorphous composition.

In a related aspect, the invention features an article including a surface coating, wherein the surface coating includes a compound of formula (A-VIII):

D1-L-D2 (A-VIII)

or a pharmaceutically acceptable salt thereof, wherein (i) each of D1 and D2 is, independently, a radical formed from a steroid; and L is a linker covalently linking D1 to D2, and (ii) at least 90% (w/w) of the surface coating is the compound of formula (A-VIII), wherein the surface coating is applied to a surface of the article by (a) depositing a solid including the compound of formula (A-VIII) on a surface to be coated, and (b) applying a heat press to the solid to form the surface coating.

In certain embodiments, (a) includes depositing a powder comprising the compound of formula (A-VIII) on the surface to be coated. In other embodiments, (a) includes forming a melt of the compound of formula (A-VIII) on the surface to be coated. In some embodiments, the solid is deposited on the surface to be coated as a powder. In some embodiments, the solid is processed into a pre-melting or an intermediate glassy state solid prior to being deposited on the surface to be coated.

In an embodiment of any of the above surface coatings or drug depots, the compound, D1, or D2 are released from the coating or drug depot through surface erosion.

In certain embodiments of any of the above surface coatings or drug depots, the surface erosion releases less than 20% (e.g., less than 18%, 15%, 12%, 10%, or 5%) of D1 or D2 (as a percentage of the total drug, D1 or D2, present in the coating or drug depot in prodrug form) at 37° C. in 100% bovine serum over 5 days, 6 days, 7 days, 8 days, 9 days, 10 days, 12 days, or more (e.g., less than 10% of D1 or D2 at 37° C. in 100% bovine serum over 5 days). In other embodiments of any of the above coatings or drug depots, the surface erosion releases less than 2.0% (e.g., less than 1.8%, 1.5%, 1.2%, 1.0%, or 0.5%) of D1 or D2 (as a percentage of the total drug, D1 or D2, present in the coating or drug depot in prodrug form) at 37° C. in PBS over 5 days, 7 days, 10 days, 14 days, or more (e.g., less than 2% of D1 or D2 at 37° C. in PBS over 5 days). In still other embodiments of any of the above coatings or drug depots, the surface erosion releases greater than 20% (e.g., greater than 22%, 24%, 26%, 28%, or 30%) of D1 or D2 (as a percentage of the total drug, D1 or D2, present in the coating or drug depot in prodrug form) at 37° C. in 100% bovine serum over not fewer than 6 days, 8 days, 10 days, or 12 days (e.g., greater than 24% of D1 or D2 at 37° C. in 100% bovine serum over 10 days). In other embodiments of any of the above coatings or drug depots, the surface erosion releases greater than 5.0% (e.g., greater than 6.0%, 8.0%, 10%, 12%, or 15%) of D1 or D2 (as a percentage of the total drug, D1 or D2, present in the coating or drug depot in prodrug form) at 37° C. in PBS over not fewer than 6 days, 8 days, 10 days, or 12 days (e.g., greater than 5% of D1 or D2 at 37° C. in PBS over 10 days). In other embodiments, the compound (D1 and/or D2) is released from the coating or drug depot at a rate such that t₁₀ is greater than or equal to 1/10 of t₅₀.

In certain embodiments, the surface coating or drug depot further includes from 0.1% to 10% (w/w) of one or more additives, in which the one or more additives are antioxidants, binders, lubricants, radio-opaque agents, and mixtures thereof.

In an embodiment of any of the above surface coatings or drug depots, the coating or drug depot has a glassy state and is formed from a compound of the disclosure.

In an embodiment of any of the above articles, L has a molecular weight of from 80 to 800 Da, e.g., 80 to 100 Da, 80 to 200 Da, 80 to 300 Da, 80 to 400 Da, 80 to 500 Da, 80 to 600 Da, or 80 to 700 Da. In another embodiment of any of the above articles, L is covalently linked to D1 and to D2 via one or more ester, carbonate, carbonate ester, or anhydride linkages. In particular embodiments, L is covalently linked to D1 and to D2 via one or more carbonate linkages.

In a particular embodiment of any of the above articles, L includes the radical —C(O)—(R^{A})—C(O)— or —O—(R^{A})—O—; R^{A} is a radical of a polyol and includes at least one free hydroxyl group or R^{A} is C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O)_{q}CH₂CH₂—, —(CH₂CH₂CH₂CH₂O)ᵣCH₂CH₂CH₂CH₂—, or —(CH₂CH(CH₃)O)ₛCH₂CH(CH₃)—; and q, r, and s are integers from 1 to 10 (e.g., 1 to 10, 1 to 5, or 5 to 10).

In an embodiment of any of the above articles, each of D1 and D2 is an anabolic steroid, an androgenic steroid, a progestin steroid, an estrogen steroid, a cancer treatment steroid, an antibiotic steroid, a glucocorticoid steroid, a benign steroid, an anti-angiogenic steroid, an intraocular pressure (IOP) lowering steroid, a cholic acid-related bile acid steroid, a cholesterol-derivative, other steroid, a pheromone, a steroid metabolite, a progestin, a neurosteroid, and a corticosteroid. In some embodiments, the steroid is a mineralocorticoid steroid. In a particular embodiment of any of the above articles, the compound is further described by one of formulas (II)-(LXXVIII), described herein. In another embodiment of any of the above articles, each of D1 and D2 is, independently, described by any one of formulas (I-a) to (I-vvv), described herein.

In the articles or drug depots of the disclosure, D1 and D2 can be formed from the same steroid, or D1 and D2 can be formed from different steroids.

In a particular embodiment of any of the above articles or drug depots, the article includes a mixture of two or more compounds of formula (A-VIII).

In one embodiment of any of the above articles or drug depots, the compound of formula (A-VIII) is further described by the formula (A-IV):

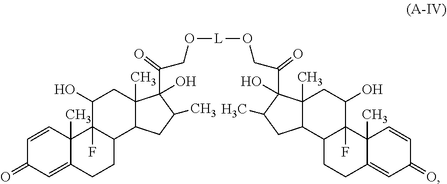

or a pharmaceutically acceptable salt thereof, in which L is —C(O)O—(R^{A})—OC(O)—; R^{A} includes C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10. In some embodiments, O—(R^{A})—O is a radical formed from an alkane diol (e.g., a C₁₋₁₀ diol), diethylene glycol, triethylene glycol, tetraethylene glycol, or pentaethylene glycol.

In an embodiment of any of the above articles or drug depots, the surface coating or drug depot is formed in or on the surface of the article or the implantable medical device and annealed. Drug depots or articles bearing surface coatings of the disclosure can be annealed by heating the compound of formula (A-VIII) above its glass transition temperature, Tg, (e.g., depending upon the compound, heating to 110-145° C., 130-185° C., 150-215° C., or 180-240° C.) for a period of from 5 minutes to 48 hours (e.g., from 5 minutes to 1 hours, from 1 to 4 hours, from 2 to 12 hours, or from 10 to 48 hours), and then cooled to form the annealed article or drug depot. In some embodiments, drug depots or articles bearing surface coatings of the disclosure can be annealed by heating the compound of formula (A-VIII) above its glass transition temperature, Tg, (e.g., depending upon the compound, heating to at least 50° C., 100° C., 150° C., 200° C., 250° C., or more (e.g., 110-145° C., 130-185° C., 150-215° C., or 180-240° C.) for a period of from 1 second to 48 hours (e.g., from 1 second to 15 minutes, from 5 minutes to 1 hour, from 1 to 4 hours, from 2 to 12 hours, or from 10 to 48 hours), and then cooled to form the annealed article or drug depot. In certain embodiments, a surface coating of a desired thickness is formed by applying multiple layers of a coating solution to the surface of an article to form a multi-layered surface coating, and the multi-layered surface coating is then annealed. The annealing of the multi-layered surface coating can reduce brittleness, and reduce the risk of cracking, flaking, or delamination of one or more of the layers. In some embodiments, the articles or drug depots provided herein do not crack, flake, delaminate, or the like.

In certain embodiments of the above articles or drug depots, the surface of the article or implantable metical device is ceramic or metallic. In other embodiments of the above articles or drug depots, the surface of the article or implantable medical device is polymeric (e.g., a surface formed from a polysilicone, polyurethane, or polyimide). In particular embodiments, the polymeric surface comprises an electrically conducting polymer. In other embodiments, the polymeric surface comprises an electrically insulating polymer.

In other embodiments of the above articles, the surface coating has a thickness between 0.5 to 120 μm (e.g., between 0.5 to 5, 1 to 10, 5 to 50, or 25 to 120 am).

The article can be a medical device and the surface coating resides on the surface of the medical device. For example, the article can be blood dwelling medical device (e.g., a heart valve, vascular stent, endovascular coil, or catheter), urine dwelling medical device (e.g., a drainage catheter or ureteral stent), and/or subcutaneously dwelling medical device (e.g., an implantable sensor). In some embodiments, the implantable medical device can be blood dwelling medical device (e.g., a heart valve, vascular stent, endovascular coil, or catheter), urine dwelling medical device (e.g., a drainage catheter or ureteral stent), and/or subcutaneously dwelling medical device (e.g., an implantable sensor). The article or implantable medical device can be an implantable device selected from prostheses pacemakers, electrical leads, defibrillators, artificial hearts, ventricular assist devices, anatomical reconstruction prostheses, artificial heart valves, heart valve stents, pericardial patches, surgical patches, coronary stents, vascular grafts, vascular and structural stents, vascular or cardiovascular shunts, biological conduits, pledges, sutures, annuloplasty rings, staples, valved grafts, dermal grafts for wound healing, orthopedic spinal implants, ophthalmic implants, intrauterine devices, maxial facial reconstruction plating, intraocular lenses, clips, and sternal wires. In particular embodiments, the implantable device is an orthopedic device selected from a wire, pin, rod, nail, screw, disk, plate, bracket, or splint. The implantable medical device can be any implantable medical device described herein. In other embodiments, the article or implantable device is selected from dental devices, drug delivery devices, grafts, stents, implantable cardioverter-defibrillators, heart valves, vena cava filters, endovascular coils, catheters, shunts, wound drains, drainage catheters, infusion ports, cochlear implants, endotracheal tubes, tracheostomy tubes, ventilator breathing tubes, implantable sensors, ophthalmic devices, orthopedic devices, dental implants, periodontal implants, breast implants, penile implants, maxillofacial implants, cosmetic implants, valves, appliances, scaffolding, suturing material, needles, hernia repair meshes, tension-free vaginal tape and vaginal slings, prosthetic neurological devices, ear tubes, a wound dressing, a bandage, a gauze, a tape, a pad, a sponge, a contraceptive device, and feminine hygiene products. In some embodiments, the implantable medical device is a subcutaneously implantable sensor.

In a related aspect, the invention features a method for administering a steroid at an implantation site in a subject, said method comprising implanting into the subject at the site (a) implantable medical device and (b) a drug depot comprising a compound of formula (A-VIII):

D1-L-D2 (A-VIII),

or a pharmaceutically acceptable salt thereof, wherein (i) each of D1 and D2 is, independently, a radical formed from a steroid; and L is a linker covalently linking D1 to D2, (ii) at least 70% (w/w) (e.g., at least 75%, 80%, 85%, 90% 92%, 94%, 96%, 98%, or 99% (w/w)) of the drug depot is the compound of formula (A-VIII), (iii) the drug depot is free of controlled release polymer, and (iv) D1 and D2 is released from the drug depot at 37° C. in 100% bovine serum or at 37° C. in PBS at a rate such that t₁₀ is greater than or equal to 1/10 of t₅₀.

In one embodiment, the drug depot is in the form of a fiber, fiber mesh, woven fabric, non-woven fabric, pellet, cylinder, hollow tube, microparticle, nanoparticle, or shaped article. In particular embodiments, the implantable medical device and the drug depot are implanted simultaneously. In certain embodiments, the method includes implanting into the subject an implantable medical device of the invention bearing a drug depot retained by or affixed to the implantable medical device. In other embodiments, the implantable medical device and the drug depot are implanted separately. In some embodiments, the drug depot is implanted at or near the site two, three, four, five, or more times over the course of one month to one year.

In an embodiment of any of the above methods, the compound, D1, or D2 are released from the drug depot through surface erosion.

In certain embodiments of any of the above methods, the surface erosion releases less than 20% (e.g., less than 18%, 15%, 12%, 10%, or 5%) of D1 or D2 (as a percentage of the total drug, D1 or D2, present in the drug depot in prodrug form) at 37° C. in 100% bovine serum over 5 days, 6 days, 7 days, 8 days, 9 days, 10 days, or 12 days (e.g., less than 10% of D1 or D2 at 37° C. in 100% bovine serum over 5 days). In other embodiments of any of the above methods, the surface erosion releases less than 2.0% (e.g., less than 1.8%, 1.5%, 1.2%, 1.0%, or 0.5%) of D1 or D2 (as a percentage of the total drug, D1 or D2, present in the drug depot in prodrug form) at 37° C. in PBS over 5 days, 7 days, 10 days, or 14 days (e.g., less than 2% of D1 or D2 at 37° C. in PBS over 5 days). In still other embodiments of any of the above methods, the surface erosion releases greater than 20% (e.g., greater than 22%, 24%, 26%, 28%, or 30%) of D1 or D2 (as a percentage of the total drug, D1 or D2, present in the drug depot in prodrug form) at 37° C. in 100% bovine serum over not fewer than 6 days, 8 days, 10 days, 12 days, or more (e.g., greater than 24% of D1 or D2 at 37° C. in 100% bovine serum over 10 days). In other embodiments of any of the above methods, the surface erosion releases greater than 5.0% (e.g., greater than 6.0%, 8.0%, 10%, 12%, or 15%) of D1 or D2 (as a percentage of the total drug, D1 or D2, present in the drug depot in prodrug form) at 37° C. in PBS over not fewer than 6 days, 8 days, 10 days, or 12 days (e.g., greater than 5% of D1 or D2 at 37° C. in PBS over 10 days). In other embodiments, the compound (D1 and/or D2) is released from the drug depot at a rate such that t₁₀ is greater than or equal to 1/10 of t₅₀.

In certain embodiments, the drug depot further includes from 0.1% to 10% (w/w) of one or more additives, in which the one or more additives are antioxidants, binders, lubricants, radio-opaque agents, and mixtures thereof.

In an embodiment of any of the above methods, the drug depot has a glassy state and is formed from a compound of the disclosure.

In an embodiment of any of the above methods, L can have a molecular weight of from 80 to 800 Da, e.g., 80 to 100 Da, 80 to 200 Da, 80 to 300 Da, 80 to 400 Da, 80 to 500 Da, 80 to 600 Da, or 80 to 700 Da. In another embodiment of any of the above methods, L is covalently linked to D1 and to D2 via one or more ester, carbonate, carbonate ester, or anhydride linkages. In particular embodiments, L is covalently linked to D1 and to D2 via one or more carbonate linkages.

In a particular embodiment of any of the above methods, L includes the radical —C(O)—(R^{A})—C(O)— or —O—(R^{A})—O—; R^{A} is a radical of a polyol and includes at least one free hydroxyl group or R^{A} is C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O)_{q}CH₂CH₂—, —(CH₂CH₂CH₂CH₂O)ᵣCH₂CH₂CH₂CH₂—, or —(CH₂CH(CH₃)O)ₛCH₂CH(CH₃)—; and q, r, and s are integers from 1 to 10 (e.g., 1 to 10, 1 to 5, or 5 to 10).

In an embodiment of any of the above methods, each of D1 and D2 is an anabolic steroid, an androgenic steroid, a progestin steroid, an estrogen steroid, a mineralocorticoid steroid, a cancer treatment steroid, an antibiotic steroid, a glucocorticoid steroid, a benign steroid, an anti-angiogenic steroid, an intraocular pressure (IOP) lowering steroid, a cholic acid-related bile acid steroid, a cholesterol-derivative, other steroid, a pheromone, a steroid metabolite, a progestin, a neurosteroid, and a corticosteroid. In a particular embodiment of any of the above methods, the compound is further described by one of formulas (II)-(LXXVIII), described herein. In another embodiment of any of the above methods, each of D1 and D2 is, independently, described by any one of formulas (I-a) to (I-vvv), described herein.

In the drug depots of the disclosure, D1 and D2 can be formed from the same steroid, or D1 and D2 can be formed from different steroids.

In a particular embodiment of any of the above methods, the drug depot includes a mixture of two or more compounds of formula (A-VIII).

In one embodiment of any of the above methods, the compound of formula (A-VIII) is further described by the formula (A-IV):

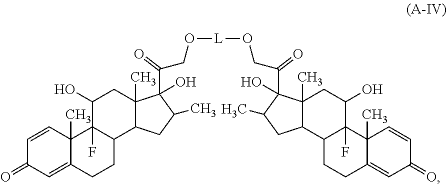

or a pharmaceutically acceptable salt thereof, in which L is —C(O)O—(R^{A})—OC(O)—; R^{A} includes C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10. In some embodiments, O—(R^{A})—O is a radical formed from an alkane diol (e.g., a C₁₋₁₀ diol), diethylene glycol, triethylene glycol, tetraethylene glycol, or pentaethylene glycol.

In certain embodiments of the methods provided herein, the surface of the implantable medical device is ceramic or metallic. In other embodiments of the above methods, the surface of the implantable medical device is polymeric (e.g., a surface formed from a polysilicone, polyurethane, or polyimide). In particular embodiments, the polymeric surface comprises an electrically conducting polymer. In other embodiments, the polymeric surface comprises an electrically insulating polymer.

The implantable medical device can be a blood dwelling medical device (e.g., a heart valve, vascular stent, endovascular coil, or catheter), urine dwelling medical device (e.g., a drainage catheter or ureteral stent), and/or subcutaneously dwelling medical device (e.g., an implantable sensor). The implantable device can be selected from prostheses pacemakers, electrical leads, defibrillators, artificial hearts, ventricular assist devices, anatomical reconstruction prostheses, artificial heart valves, heart valve stents, pericardial patches, surgical patches, coronary stents, vascular grafts, vascular and structural stents, vascular or cardiovascular shunts, biological conduits, pledges, sutures, annuloplasty rings, staples, valved grafts, dermal grafts for wound healing, orthopedic spinal implants, ophthalmic implants, intrauterine devices, maxial facial reconstruction plating, intraocular lenses, clips, and sternal wires. In particular embodiments, the implantable device is an orthopedic device selected from a wire, pin, rod, nail, screw, disk, plate, bracket, or splint. The implantable medical device can be any implantable medical device described herein. In other embodiments, the implantable device is selected from dental devices, drug delivery devices, grafts, stents, implantable cardioverter-defibrillators, heart valves, vena cava filters, endovascular coils, catheters, shunts, wound drains, drainage catheters, infusion ports, cochlear implants, endotracheal tubes, tracheostomy tubes, ventilator breathing tubes, implantable sensors, ophthalmic devices, orthopedic devices, dental implants, periodontal implants, breast implants, penile implants, maxillofacial implants, cosmetic implants, valves, appliances, scaffolding, suturing material, needles, hernia repair meshes, tension-free vaginal tape and vaginal slings, prosthetic neurological devices, ear tubes, and a wound dressing. In particular embodiments, the implantable medical device is a subcutaneously implantable sensor.

In some embodiments, the method includes ameliorating inflammation at an implantation site in a subject, wherein the compound of formula (A-VIII) is further described by the formula (A-IV):

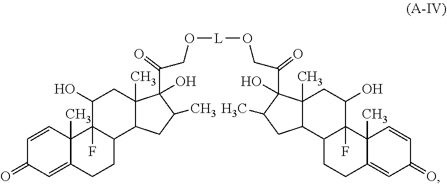

or a pharmaceutically acceptable salt thereof, in which L is —C(O)O—(R^{A})—OC(O)—; R^{A} includes C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10. In some embodiments, O—(R^{A})—O is a radical formed from an alkane diol (e.g., a C₁₋₁₀ diol), diethylene glycol, triethylene glycol, tetraethylene glycol, or pentaethylene glycol. In particular embodiments, the implantable medical device is a subcutaneously implantable sensor. In certain embodiments, the method includes implanting into the subject an implantable medical device of the invention bearing a drug depot retained by or affixed to the implantable medical device. In one embodiment, the implantable medical device includes a reservoir or holder for retaining the drug depot. The reservoir can include a drug depot in the form of a fiber, fiber mesh, woven fabric, non-woven fabric, pellet, cylinder, hollow tube, microparticle, nanoparticle, or shaped article. In particular embodiments, the holder includes a drug depot in the form of a sheath, collar, ring, washer, fibrous pouch, or threaded shaped article configured for placement within or upon the holder such that the drug depot is retained by the implantable medical device. In other embodiments, the drug depot is adhesively affixed to said implantable medical device. The drug depot adhesively affixed to the implantable medical device can be in the form of a fiber, fiber mesh, woven fabric, non-woven fabric, wafer, sheet, film, pellet, cylinder, hollow tube, microparticle, nanoparticle, or shaped article. In other embodiments, the drug depot is not adhesively affixed and is held by the device through mechanical components (e.g. screw, nut, bolt, washers) or becomes a part of the device on its own (e.g. a fibrous pouch around the device).

The invention further features a method for making an implantable medical device bearing a drug depot retained by or affixed to the implantable medical device, the method including (i) providing the implantable medical device and the drug depot; and (ii) affixing the drug depot to the implantable medical device. For example, the depot can be affixed to the implantable medical device by (a) applying heat or an organic solvent to the surface of the depot to form a sticky surface; and (b) contacting the sticky surface to the implantable medical device to affix the depot to the implantable medical device. In some embodiments, the depot is in the form of a film affixed to the implantable medical device. In another embodiment, the depot is affixed to the implantable medical device by electrospinning fibers of the depot onto a surface of the implantable medical device. In some embodiments, the affixed fibers circumscribe the implantable medical device. In some embodiments, the depot and a polymeric portion of the implantable medical device are heat processed and co-extruded to form a depot affixed the surface of the polymeric portion of the implantable medical device.

Provided herein is a method of forming a surface coating on an article of the invention, the method including: (a) dissolving a compound of formula (A-VIII):

D1-L-D2 (A-VIII),

or a pharmaceutically acceptable salt thereof, in a solvent to form a solution, wherein each of D1 and D2 is, independently, a radical formed from a steroid; and L is a linker covalently linking D1 to D2, (b) applying the solution to the surface of the article, (c) evaporating solvent on the surface of the article to form the surface coating, and (d) repeating (b) and (c).

In a related aspect, provided herein is a method of forming a surface coating on an article of the invention, the method including: (a) dissolving a compound of formula (A-VIII):

D1-L-D2 (A-VIII),

or a pharmaceutically acceptable salt thereof, in a solvent to form a solution, wherein each of D1 and D2 is, independently, a radical formed from a steroid; and L is a linker covalently linking D1 to D2, (b) applying the solution to the surface of the article, (c) evaporating solvent on the surface of the article to form the surface coating, and (d) annealing the surface coating. In an embodiment of any of the above articles, the surface coating is formed on the surface of the article and annealed. Articles bearing surface coatings of the disclosure can be annealed by heating the compound of formula (A-VIII) above its glass transition temperature, Tg, (e.g., depending upon the compound, heating to 110-145° C., 130-185° C., 150-215° C., or 180-240° C.) for a period of from 5 minutes to 48 hours (e.g., from 5 minutes to 1 hours, from 1 to 4 hours, from 2 to 12 hours, or from 10 to 48 hours), and then cooled to form the annealed article.

In certain embodiments, provided herein is a method of forming a surface coating on an article of the invention, the method including: (a) dissolving a compound of formula (A-VIII):

D1-L-D2 (A-VIII),

or a pharmaceutically acceptable salt thereof, and (b) dissolving a plasticizing agent in a solvent to form a solution, wherein each of D1 and D2 is, independently, a radical formed from a steroid; and L is a linker covalently linking D1 to D2, (c) applying the solution to the surface of the article, (d) evaporating solvent on the surface of the article to form the surface coating. In particular embodiments, the one or more plasticizing agents are selected from glycerol, ethylene glycol, diethylene glycol, triethylene glycol, tetraethylene glycol, polyethylene glycol, propylene glycol, triacetin, sorbitol, mannitol, xylitol, fatty acids, monosaccharides (e.g., glucose, mannose, fructose, sucrose), ethanolamine, urea, triethanolamine, vegetable oils, lecithin, and waxes.

In any of the methods provided herein the solvent can be selected from tetrahydrofuran, N,N-dimethylformamide, diethylamine, dichloromethane, chloroform, methyl t-butyl ether, toluene, benzene, ether, p-xylene, carbon disulfide, carbon tetrachloride, cyclohexane, pentane, hexane, heptane, dioxane, ethylacetate, dimethoxyethane, ethyl benzoate, anisol, chlorobenzene, pyridine, acetone, dimethylsulfoxide, acetonitrile, ethanol, n-propanol, toluene, methanol, benzyl alcohol, and mixture thereof.

The concentration of the compound of formula (A-VIII) in the solution can be between 10 and 250 mg/mL (e.g., between 10 and 50, 25 and 75, 60 and 120, 80 and 150 mg/mL, or 125 and 250 mg/mL). In some embodiments, (b) and (c) are repeated from 2 to 100 times (e.g., repeated 2 to 25, 10 to 50, 20 to 75, or 40 to 100 times). (b) can include dip coating, drop coating, drop and drag coating, or spray coating the solution onto the surface of the article or electrospinning or electrospraying the solution to form the surface coating. In certain embodiments, (c) includes evaporating the solvent to form the surface coating having a glassy state composition.

In certain embodiments, the invention features a method of forming a surface coating on an article of the invention, the method including: (a) placing a powder comprising a compound of formula (A-VIII):

D1-L-D2 (A-VIII),

or a pharmaceutically acceptable salt thereof, onto the surface of an article; and (b) annealing the powder (e.g., amorphous solid) to form the surface coating, wherein each of D1 and D2 is, independently, a radical formed from a steroid; and L is a linker covalently linking D1 to D2. In certain embodiments, the invention features a method of forming a surface coating on an article of the invention, the method including: (a) placing a powder comprising a compound of formula (A-VIII):

D1-L-D2 (A-VIII),

or a pharmaceutically acceptable salt thereof, onto the surface of an article; and (b) melting the powder (e.g., crystalline solid) to form the surface coating, wherein each of D1 and D2 is, independently, a radical formed from a steroid; and L is a linker covalently linking D1 to D2. In particular embodiments, the powder further includes one or more plasticizing agents selected from glycerol, ethylene glycol, diethylene glycol, triethylene glycol, tetraethylene glycol, polyethylene glycol, propylene glycol, triacetin, sorbitol, mannitol, xylitol, fatty acids, monosaccharides (e.g., glucose, mannose, fructose, sucrose), ethanolamine, urea, triethanolamine, vegetable oils, lecithin, and waxes. In some embodiments, the powder coating and annealing are repeated from 2 to 100 times (e.g., repeated 2 to 25, 10 to 50, 20 to 75, or 40 to 100 times).

In certain embodiments, provided herein is a method of forming a surface coating on an article of the invention, the method including: (a) providing a compound of formula (A-VIII):

D1-L-D2 (A-VIII),

or a pharmaceutically acceptable salt thereof, to a surface, wherein each of D1 and D2 is, independently, a radical formed from a steroid; and L is a linker covalently linking D1 to D2, (b) depositing the compound on a surface of the article, and (c) applying a heat press to the solid to form the surface coating. In some embodiments, the compound is a solid. In some embodiments, the solid is applied directly to the surface. In some embodiments, the solid is dissolved in a solvent, a solution, or the like. In some embodiments, the solvent, solution, or the like is applied to the surface. In some embodiments, the solvent is evaporated to provide the solid on the surface (e.g., and then the solvent is heat pressed to form the surface coating).

In certain embodiments, provided herein is a method of forming a surface coating on an article of the invention, the method including: (a) providing a solid including a compound of formula (A-VIII):

D1-L-D2 (A-VIII),

or a pharmaceutically acceptable salt thereof, in a solvent to form a solution, wherein each of D1 and D2 is, independently, a radical formed from a steroid; and L is a linker covalently linking D1 to D2, (b) depositing the solid on a surface of the article, and (c) applying a heat press to the solid to form the surface coating.

In certain embodiments, (b) includes depositing a powder including the compound of formula (A-VIII) on the surface. In other embodiments, (b) includes heating the solid to form a melt of the compound of formula (A-VIII) on the surface. In some embodiments, at least 90% (w/w) (e.g., at least 92%, 94%, 96%, 98%, or 99% (w/w)) of the solid is the compound of formula (A-VIII). Optionally, following (c), the surface coating is annealed. In some embodiments of the above method, (c) includes applying heat and pressure to the solid for less than 10 minutes, 5 minutes, 3 minutes, 2 minutes, 1 minute, or 30 seconds. In some embodiments, (c) includes applying heat and pressure to the solid for more than 10 mins, 20 mins, or more. In some embodiments, (c) includes applying heat and pressure to the solid for less than 10 minutes, 5 minutes, 3 minutes, 2 minutes, 1 minute, 30 seconds, or less at a temperature of more than about 50° C., 100° C., 110° C., 120° C., 130° C., 140° C., 150° C., 160° C., 170° C., 180° C., 190° C., 200° C., 250° C., or more. In some embodiments, (c) includes applying heat and pressure to the solid for less than 5 minutes, 3 minutes, 2 minutes, 1 minute, 30 seconds, or less at a temperature of more than about 150° C., 160° C., 170° C., 180° C., or more In some embodiments, (c) includes applying heat and pressure to the solid for less than 30 seconds. For example, (c) can include heating the solid to at least 150° C. (e.g., depending upon the compound, heating to 150-215° C. or 180-240° C.). (c) can include placing the solid under a pressure of at least 500 PSI (e.g., at least 500 PSI, at least 600 PSI, at least 700 PSI, at least 800 PSI, at least 900 PSI, at least 1,000 PSI, at least 1,500 PSI, at least 2,000 PSI, at least 2,500 PSI, at least 3,000 PSI, or more (e.g., from about 500 PSI to about 3,000 PSI, about 1,000 PSI to about 2,000 PSI, or about 1,000 PSI to about 1,500 PSI)). In certain embodiments, the surface of the article is ceramic, polymeric, or metallic. In some embodiments, the surface coating has a thickness between 0.5 to 120 μm (e.g., between 0.5 to 5, 1 to 10, 5 to 50, or 25 to 120 am).

In some embodiments, the annealing process is performed with heat. In some embodiments, the annealing process is performed with pressure. In some embodiments, the annealing process is performed with heat and pressure. In some embodiments, the annealing process is performed with heat and pressure on flat surfaces. In some embodiments, the annealing process is performed with heat on flat, oblong, spherical, cubic, polygonic, or the like surfaces.

In some embodiments, the compound, D1, or D2 are released from the coating through surface erosion.

Provided in some embodiments herein is a surface coating having a glassy state formed from a compound of the disclosure.

Provided in some embodiments herein is an implantable medical device including a coating of the disclosure, in which the coating resides on the surface of the implantable medical device.

In another embodiment of any of the above articles, depots, and/or methods, the compound is further described by one of formulas (II)-(LXXVIII), described herein.

In still another embodiment of any of the above articles, depots, and/or methods, R^{A} is —(CH₂CH₂O)_{q}CH₂CH₂—, q is an integer of 1 to 10, and upon hydrolysis each of D1 and D2, independently, form dexamethasone, triamcinolone, betamethasone, prednisolone, prednisone, fluocinolone, fluocinolone acetonide, mometosone, mometosone furoate, anecortave, hydrocortisone, triamcinolone acetonide, methylprednisolone, budesonide, fusidic acid, aldosterone, or fludrocortisone.

In some embodiments, the compound is processed as described herein (e.g., melt processed or solvent processed) to form a glassy state solid. The glassy state solid is subsequently heated above its glass transition temperature, Tg, and annealed for a period of from 5 minutes to 48 hours (e.g., from 5 minutes to 1 hours, from 1 to 4 hours, from 2 to 12 hours, or from 10 to 48 hours) followed by cooling.

In some embodiments (e.g., of the articles, drug depots, and methods provided herein), the article or drug depot is free of controlled release excipient.

In some embodiments (e.g., of the articles, drug depots, and methods provided herein), the article or drug depot is free of a crystallization inhibiting excipient

In some embodiments (e.g., of the articles, drug depots, and methods provided herein), the article or drug depot is free of a binding excipient.

In some embodiments (e.g., of the articles, drug depots, and methods provided herein), the surface coating is a patterned coating on the surface of the article selected from a checkerboard pattern, dot pattern, or striped pattern. In other embodiments, the article has two or more sides and at least one of the sides contains no surface coating.

In some embodiments (e.g., of the articles, drug depots, and methods provided herein), the method includes masking a portion or side of the article to produce a single-sided or patterned coating (e.g., a checkerboard pattern, dot pattern, or striped pattern).

### Definitions

The term “annealing,” as used herein, refers to the process of heating a surface coating or drug depot formed from the compound of formula (A-VIII) above its glass transition temperature, Tg, (e.g., depending upon the compound, heating to at least 50° C. (e.g., 110-145° C., 130-185° C., 150-215° C., or 180-240° C.) for a period sufficient to reduce brittleness, or reduce the risk of cracking, flaking, or delamination of the surface coating or drug depot (e.g., for at least 1 second (e.g., from 5 minutes to 48 hours) followed by cooling. In some embodiments, the period sufficient to reduce brittleness, or reduce the risk of cracking, flaking, or delamination of the surface coating is less than 5 minutes (e.g., less than 5 minutes, less than 4 minutes, less than 3 minutes, less than 2 minutes, less than 1 minute, less than 30 seconds, or less than 5 seconds).

The term “heat press,” as used herein, refers to the process of simultaneously heating and pressing (e.g. adding pressure to) a solid provided herein (e.g., including the compound of formula (A-VIII)) to form a surface coating. For example, the solid can be heated to a temperature greater than 50° C. (e.g., depending upon the compound, heating to 150-215° C. or 180-240° C.), while simultaneously applying pressure to the solid (500-3000 PSI). The process of applying a heat press can dramatically increase drug loadings as described in Example 13. The process of applying a heat press can dramatically improve coating integrity and reduce defects, e.g., as shown in FIGS. 16A, 16B, and 17.

The term “free of controlled release polymer,” as used herein, generally refers to the absence (e.g., less than 5 wt. %, less than 2 wt. %, less than 1 wt. %) of an amount of a polymeric material of greater than 10 KDa in the surface coatings or drug depots provided herein that is sufficient to delay or slow the release of the steroid dimer from the surface coating or drug depot in comparison to the release profile observed for an otherwise identical surface coating or drug depot containing none of the polymeric material, (e.g., where the release profile is measured at 37° C. in 100% fetal bovine serum (FBS)).

The term “free of a crystallization inhibiting excipient,” as used herein, generally refers to the absence (e.g., less than 5 wt. %, less than 2 wt. %, less than 1 wt. %) of an amount of an excipient in the surface coatings or drug depots of the disclosure that is sufficient to reduce the amount of crystalline steroid dimer in the surface coating or drug depot in comparison to the amount of crystalline steroid dimer observed in an otherwise identical surface coating or drug depot containing none of the excipient. The level of crystallinity can be measured using DSC or XRD. In some embodiments, the surface coatings or drug depots of the disclosure are free of a crystallization inhibiting excipient that is a polymeric material of greater than 10 KDa.

The term “free of a binding excipient,” as used herein, generally refers to the absence (e.g., less than 5 wt. %, less than 2 wt. %, less than 1 wt. %) of an amount of an excipient in the surface coatings or drug depots of the disclosure that is sufficient to delay or slow the release of the steroid dimer from the surface coating or drug depot in comparison to the release profile observed for an otherwise identical surface coating or drug depot containing none of the binding excipient, where the release profile is measured at 37° C. in 100% FBS.

The term “anti-angiogenic steroid” refers to a steroid that halts the process of developing new blood vessels (i.e., angiogenesis). Examples of anti-angiogenic steroids include anecortave acetate, anecortave, 11-epicortisol, 17α-hydroxyprogesterone, tetrahydrocortexolone, and tetrahydrocortisol.

The term “benign steroid” as used herein, refers to low glucocorticoid activity and low mineral corticoid activity. Benign steroids include, without limitation, cholesterol, bile acids (such as cholic acid), and phytosterols (such as beta-sitosterol). Exemplary benign steroids include cholesterol, 11-deoxycortisol, 11-deoxycorticosterone, pregnenolone, cholic acid, chenodeoxycholic acid, ursodeoxycholic acid, obeticholic acid, tetrahydrocortisone, tetrahydrodeoxycortisol, tetrahydrocorticosterone, 5α-dihydrocorticosterone, and 5α-dihydropregesterone.

The term “cholesterol-derivative” refers to steroids that are derived from cholesterol. Examples of cholesterol-derivatives are 22R-hydroxycholesterol, and 20α-22R-dihydroxycholesterol.

The term “cholic acid-related bile acid steroid” refers to a steroid that is derived from cholic acid. Examples of cholic acid-related bile acid steroids are deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, and tauroursodeoxycholic acid.

The term “cylinder,” as used herein, refers to the shape of the drug depots of the disclosure that has parallel sides and a circular or oval cross section, or a shaped cross section (e.g., a star shaped cross section). A mean diameter of the cylinder can range from about 0.01 to 1 mm diameter, e.g., about 0.01 to 0.2 mm, about 0.1 to 0.3 mm, about 0.1 to 0.4 mm, about 0.2 to 0.5 mm, about 0.1 to 0.6 mm, about 0.1 to 0.7 mm, about 0.1 to 0.8 mm, or about 0.1 to 0.9 mm. A mean length of the cylinder can range from about 0.05 to 20 mm, e.g., about 0.05 to 1 mm, about 0.5 to 2 mm, about 0.5 to 4 mm, about 0.5 to 6 mm, about 0.5 to 8 mm, about 0.5 to 10 mm, about 0.5 to 12 mm, about 0.5 to 14 mm, about 0.5 to 16 mm, or about 0.5 to 18 mm. In some embodiments, the mean diameter of the cylinder is in the range of about 0.01 to 1 mm and the mean length of the cylinder is about 0.1 mm to 4.0 mm. In some embodiments, the mean length of the cylinder is about 0.5 to 10 mm, or about 1 to 10 mm.

The term “fiber,” as used herein, refers to the shape of the drug depots of the disclosure that is elongated or threadlike. A mean diameter of the fiber can range from about 0.01 to 1 mm, e.g., 0.05 to 0.3 mm, 0.1 to 0.3 mm, 0.15 to 0.3 mm, 0.2 to 0.3 mm, 0.25 to 0.3 mm, 0.01 to 0.1 mm, 0.01 to 0.2 mm, 0.01 to 0.3 mm, 0.01 to 0.4 mm, 0.01 to 0.5 mm, 0.01 to 0.6 mm, 0.01 to 0.7 mm, 0.01 to 0.8 mm, or 0.01 to 0.9 mm. A mean length of the fiber can range from about 20 to 20,000 mm, e.g., about 20 to 1000 mm, about 20 to 2,000 mm, about 100 to 2,000 mm, about 100 to 5,000 mm, about 1,000 to 8,000 mm, about 2,000 to 8,000 mm, about 2,000 to 10,000 mm, about 2,000 to 12,000 mm, about 2,000 to 15,000 mm, or about 5,000 to 18,000 mm.

The term “fiber mesh,” as used herein refers to a web or a net in having at least one attached or woven fibers. The fiber mesh can have aligned and unaligned morphologies.

The term “glassy state,” as used herein, refers to an amorphous solid including greater than 70%, 80%, 90%, 95%, 98%, or 99% (w/w) of one or more drug dimers of the disclosure and exhibiting a glass transition temperature in the range of from 38 to 150° C. In some embodiments, the glassy state temperature of a compound described herein exhibits a glass transition temperature of greater or equal to 38° C. In some embodiments, the glassy state temperature of a compound described herein exhibits a glass transition temperature of greater or equal to 150° C. In the glassy state, as measured by DSC or XRD, the level of crystallinity is low, ranging from 0-15%, e.g., 0-1%, 0-3%, 0-5%, 0-7%, 0-9%, 0-10%, or 0-13%. Glass formulations of the disclosure can be formed using heat processing or solvent processing one or more drug dimers.

The term “intraocular pressure (IOP) lowering steroid” refers to a steroid that lowers the intraocular pressure. Examples of intraocular pressure (IOP) lowering steroids are anecortave acetate, anecortave, 11-epicortisol, 17α-hydroxyprogesterone, tetrahydrocortexolone, and tetrahydrocortisol.

The term “microparticle,” as used herein, refers to the shape of the drug depots of the disclosure, which can be regularly or irregularly shaped. A mean diameter of the microparticle can range from about 1 to 1000 μm, e.g., about 10 to 1000 μm, about 100 to 1000 am, about 200 to 1000 μm, about 500 to 1000 μm, about 700 to 1000 μm, or about 900 to 1000 μm. As used herein, a “microbead” refers to a microparticle that is spherical.

The term “mineralocorticoid steroid” refers to a steroid that can influence salt balance in the body. Examples of mineralocorticoid steroids are fludrocortisone and aldocortisone.

The term “nanoparticle,” as used herein, refers to the shape of the drug depots of the disclosure, which can be regularly or irregularly shaped. A mean diameter of the nanoparticle can range from about 0.01 to 1 μm, e.g., about 0.05 to 1 μm, about 0.1 to 1 μm, about 0.2 to 1 μm, about 0.3 to 1 μm, about 0.4 to 1 μm, about 0.5 to 1 μm, about 0.6 to 1 μm, about 0.7 to 1 μm, about 0.8 to 1 μm, or about 0.9 to 1 μm. As used herein, a “nanobead” refers to a nanoparticle that is spherical.

The term “neurosteroid” refers to an endogenous or exogenous steroid that rapidly alters neuronal excitability through interaction with ligand-gated ion channels and other cell surface receptors. Exemplary neurosteroids are alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, and 24(S)-hydroxycholesterol.

The term “non-woven fabric,” as used herein, refers to a web structure bonded together by entangling fibers.

The term “other steroid” refers to a compound that has a steroid-based structure. Examples of the steroids are flugestone, prebediolone, chlormadinone acetate, medrogestone, and segesterone acetate.

The term “pellet,” as used herein, refers to the shape of the pharmaceutical compositions of the disclosure that is rounded, spherical, or cylindrical, or a combination thereof. A mean diameter of the pellet can range from about 0.2 to 5 mm, e.g., from about 0.2 to 1 mm, from about 0.2 to 2 mm, from about 0.3 to 3 mm, from about 1.5 to 5 mm, from about 2 to 5 mm, from about 2.5 to 5 mm, from about 3 to 5 mm, from about 3.5 to 5 mm, from about 4 to 5 mm, or from about 4.5 to 5 mm.

The term “pharmaceutically acceptable salt” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describe pharmaceutically acceptable salts in detail in J. Pharm. Sci. 66:1-19, 1977. The salts can be prepared in situ during the final isolation and purification of the compounds of the disclosure or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, carbonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pheromone” refers to a steroid hormone. Examples of pheromones are androstadienol, androstadienone, androstenol, androstenone, estratetraenol, 5-dehydroprogesterone, 6-dehydro-retroprogesterone, allopregnanolone, and hydroxyprogesterone caproate.

The term “steroid metabolite” refers to a product of metabolism of a steroid. Examples of steroid metabolites are tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, and progesterone.

The term “progestin” refers to a natural or synthetic steroid hormone. Examples of progestins are allopregnone-3α,20α-diol, allopregnone-3β,20β-diol, allopregnane-3μ,21-diol-11,20-dione, allopregnane-3β,17α-diol-20-one, 3,20-allopregnanedione, 3β,11β,17a, 20β,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,20β-triol, allopregnane-3β,17α,21-triol-11,20-dione, allopregnane-3β,11β,21-triol-20-one, allopregnane-3β,17α,21-triol-20-one, allopregnane-3α-ol-20-one, allopregnane-3β-ol-20-one, pregnanediol, 3,20-pregnanedione, 4-pregnene-20,21-diol-3,11-dione, 4-pregnene-11β,17α,20β,21-tetrol-3-one, 4-pregnene-17α,20β,21-triol-3,11-dione, 4-pregnene-17α,20β,21-triol-3-one, and pregnenolone.

The term “surface erosion,” as used herein refers to a process of a gradual disintegration or dissolution of the articles, depots, compounds, and/or pharmaceutical compositions provided herein. In some embodiments, surface erosion is a measure of release of a free drug from the drug dimer. Surface erosion can be tailored to achieve desired drug release rates. Surface erosion can depend on the drug composition of the drug dimer, and can be modulated by the cleavage of drug-linker bond through hydrolysis and/or enzymatic degradation. The rate of surface erosion and release of a given drug from a drug dimer may also depend on the quantity of the loaded drug dimer as a percent of the final drug dimer formulation, surface coating or drug depot thickness, solubility of drug dimer (e.g., through selection of appropriate drug and/or linker), and/or surface area of the surface coating or drug depot. For example, surface erosion mechanism of drug release allows implantable devices to be tailored with specific physical features (dimensions, diameters, surface areas, total mass, etc.) to achieve desired drug release rates from the surface coatings or drug depots, and drug release may be designed to be initiated within minutes or hours, and may continue to occur over days, weeks, months, or years post implantation.

As stated herein, “t₅₀” is the time at which 50% of the releasable drug has been released from a surface coating or drug depot of the disclosure. Time t₁₀ is, correspondingly, the time at which 10% of the releasable drug has been released from a surface coating or drug depot of the disclosure. When the release curve is perfectly linear, t₁₀=⅕ of t₅₀. When there is an initial burst of released drug, t₁₀ is much less than ⅕ of t₅₀. In the compositions and methods of the disclosure t₁₀ can be equal to or greater than 1/10 of t₅₀. Drug release from a surface coating, drug depot, or compound of the disclosure can be measured at 37° C. in 100% bovine serum, or at 37° C. in PBS (phosphate buffered saline), as described in Example 1.

### Chemical Definitions

By “acyl” is meant a chemical moiety with the formula —C(O)R′, where R′ is selected from the group consisting of C₁₋₁₀ alkyl, C₂₋₂₀ alkene, heteroalkyl, C₂₋₂₀ alkyne, C₅₋₁₀ aryl, and cyclic system. Examples of acyl groups include, without limitation, acetyl, propanoyl, butanoyl, pentanoyl, and tetrahydrofuran-2-oyl.

By “aliphatic” is meant a non-aromatic chemical moiety of hydrocarbons.

Aliphatics may be cyclic, straight, or branched chains, and may be saturated or unsaturated, and may have single, double, or triple bonds.

By “alkoxy” is meant a chemical substituent of the formula —OR, wherein R is an alkyl group. By “aryloxy” is meant a chemical substituent of the formula —OR, wherein R is a C₅₋₁₀ aryl group.

As used herein, the terms “alkylene,” “alkenylene,” “alkynylene,” and the prefix “alk” refer to divalent groups having a specified size, typically C₁₋₁₀ or C₁₋₂₀ for the saturated groups (e.g., alkylene or alk) and C₂₋₂₀ or C₂₋₂₀ for the unsaturated groups (e.g., alkenylene or alkynylene). They include straight-chain, branched-chain, and cyclic forms as well as combinations of these, containing only C and H when unsubstituted. Because they are divalent, they can link together two parts of a molecule. Examples are methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a C1 alkylene that is substituted by ═O, for example.

By “alkylthio” is meant a chemical substituent of the formula —SR, wherein R is an alkyl group.

By “arylthio” is meant a chemical substituent of the formula —SR, wherein R is a C₅₋₁₀ aryl group.

By “C₁₋₂₀ alkyl” is meant a branched or unbranched saturated hydrocarbon group, having 1 to 20 carbon atoms, inclusive. An alkyl may optionally include monocyclic, bicyclic, or tricyclic rings, in which each ring desirably has three to six members. The alkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₂₀ alkene” is meant a branched or unbranched hydrocarbon group containing one or more double bonds, desirably having from 2 to 10 carbon atoms. A C₂₋₂₀ alkene may optionally include monocyclic, bicyclic, or tricyclic rings, in which each ring desirably has five or six members. The C₂₋₂₀ alkene group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “C₂₋₂₀ alkyne” is meant a branched or unbranched hydrocarbon group containing one or more triple bonds, desirably having from 2 to 10 carbon atoms. A C₂₋₂₀ alkyne may optionally include monocyclic, bicyclic, or tricyclic rings, in which each ring desirably has five or six members. The C₂₋₂₀ alkyne group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “carbonate ester” is meant a linkage group having the formula —C(O)O—C(O)—O—.

By “carboxyalkyl” is meant a chemical moiety with the formula —(R)—COOH, wherein R is an alkyl group.

By “cyclic acetal” is meant a ring structure including two oxygen atoms separated by a carbon atom which is optionally substituted (e.g., 1,3-dioxolane). Exemplary substituents include, without limitation, alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, quaternary amino, phosphodiester, phosphoramidate, phosphate, phosphonate, phosphonate ester, sulfonate, sulfate, sulfhydryl, phenol, amidine, guanidine, and imidazole groups.

The term “cyclic system” refers to a compound that contains one or more covalently closed ring structures, in which the atoms forming the backbone of the ring are composed of any combination of the following: carbon, oxygen, nitrogen, sulfur, and phosphorous. The cyclic system may be substituted or unsubstituted. Exemplary substituents include, without limitation, alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

By “fluoroalkyl” is meant an alkyl group that is substituted with a fluorine.

By “heteroalkyl” is meant a branched or unbranched alkyl group in which one or more methylenes (—CH₂—) are replaced by nitrogen, oxygen, sulfur, carbonyl, thiocarbonyl, phosphoryl, or sulfonyl moieties. Some examples include tertiary amines, ethers, thioethers, amides, thioamides, carbamates, thiocarbamates, phosphoramidates, sulfonamides, and disulfides. A heteroalkyl may optionally include monocyclic, bicyclic, ortricyclic rings, in which each ring desirably has three to six members. The heteroalkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “hydroxyalkyl” is meant a chemical moiety with the formula —(R)—OH, wherein R is an alkyl group.

### BRIEF DESCRIPTION OF THE DRAWINGS

FIG. 1 is the structure of a composition provided herein (e.g., Compound 1 (dexamethasone-triethylene glycol-dexamethasone, Dex-TEG-Dex)).

FIG. 2A-2C shows a series of substrates (SIBS, Titanium, and Dacron) uncoated, coated with a composition provided herein (e.g., compound 1), or coated with free dexamethasone.

FIG. 3 is a graph showing drug release from the coated surfaces shown in FIGS. 2B & 2C as well as a composition provided herein (e.g., compound 1) and dexamethasone coated onto a glass substrate.

FIG. 4 is a graph showing a composition provided herein (e.g., compound 1) coating retention on different substrates after drug release a buffered solution.

FIG. 5 is a graph showing loading of a composition provided herein (e.g., Compound 1) on substrates with surface areas of 1 cm².

FIG. 6 is a graph showing loading of a composition provided herein (e.g., Compound 1) on substrates with surface areas of 3-6 mm².

FIG. 7 is a graph showing loading of a composition provided herein (e.g., Compound 1) on substrates after dip coating.

FIG. 8 is a graph showing loading of a composition provided herein (e.g., Compound 1) on substrates after spray coating.

FIG. 9 is a graph showing the percent of a composition provided herein (e.g., Compound 1) spray coating retained following a 24-hour incubation in a buffered solution at 37° C.

FIG. 10A-10B shows a series of graphs showing purity and drug release from a composition provided herein (e.g., Compound 1) coated onto fibrous meshes pre- and post-sterilization.

FIG. 11 is an image of a composition provided herein (e.g., Compound 1) electrosprayed and annealed onto a polymeric surface.

FIG. 12A-12B shows a series of images of a composition provided herein (e.g., Compound 1) coated onto an angioplasty balloon and a cardiac stent.

FIG. 13A-13B shows the structure of a composition provided herein (e.g., Compound 2 (hydrocortisone-triethylene glycol-hydrocortisone; HC-TEG-HC)) and drug release from a coated surface of a substrate.

FIG. 14A-14B shows the structure of a composition provided herein (e.g., Compound 3 (triamcinolone acetonide-triethylene glycol-triamcinolone acetonide; TA-TEG-TA)) and drug release from a coated surface of a substrate.

FIG. 15A-B shows the structure of a composition provided herein (e.g., Compound 4 (dexamethasone-hexane-dexamethasone; Dex-HEX-Dex)) and drug release from a coated surface of a substrate.

FIG. 16A-B shows a side by side comparison of heat press and spray coating methods.

FIG. 17 shows heat press coatings at different surface areas and similar drug densities.

FIG. 18A-B shows the fibrotic response and drug release profiles between dexamethasone coated discs versus compositions provided herein (e.g., Compound 1 and Compound 4) coated discs. A reduced response correlates to sustained drug release.

FIG. 19A-19B injectable cylinders of an exemplary conjugated provided herein (e.g., Compound 1) and in vitro drug release from the injectable cylinders.

FIG. 20A-20B show fibrous meshes of compositions provided herein (e.g., Compound 1). FIG. 20C depicts the differential scanning calorimetry (DSC) analysis of compositions provided herein (e.g., Compound 1) as the starting powder and solvent-processed fibrous mesh. FIG. 20D depicts the power x-ray diffraction (PXRD) analysis of compositions provided herein (e.g., Compound 1) as the starting powder and solvent-processed fibrous mesh.

FIG. 21A-21C shows cylinders of compositions provided herein (e.g., Compound 1) and sutures injected adjacent to each other in the subcutaneous tissue of rats and a graph showing drug release from the cylinders.

### DETAILED DESCRIPTION

While the clinical importance of sustained drug release delivery systems to maintain therapeutic concentration of drugs for extended periods of time (e.g., days to weeks, to months or even years) has been well acknowledged for decades, there has been a limited number of successfully commercialized products on the market to date. It is recognized herein that to develop sustained drug delivery systems, technical difficulties must be overcome, such as, for example, drug degradation during formulation process; lack of controlled release, including unwanted burst or incomplete release associated with diffusion or bulk erosion mechanisms of drug release; low encapsulation efficiency; and formulation complexity. Achieving long linear release profiles can be particularly difficult where the drug release system is entirely contained within a surface coating or drug depot.

Provided herein are surface coatings and drug depots formed from dimers that are processable as solids or liquids (e.g., from a melt or solution). In some embodiments, the solids or liquids are used to coat articles, such as medical devices. In some embodiments, most of the material in the surface coating is optionally in a glassy state. The surface coatings can provide a controlled rate of drug release over days, weeks, months, or years, due to interactions between the molecules that exist in a mostly amorphous state while holding the shaped form intact as the surface erodes. This disclosure also describes drug depots for use in combination with implantable medical devices. In some embodiments, the drug depots are formed from dimers. The drug depots can provide a controlled rate of drug release over days, weeks, months, or years, due to interactions between the molecules that exist in a mostly amorphous state while holding the shaped form intact as the surface erodes. In some embodiments, surface coatings and drug depots provided herein minimize inflammatory responses (e.g., because the drugs/prodrugs undergoing surface erosion from the article or drug depot can be released in the biological environment in a non-particulate (e.g., non-crystalline) form) In certain instances, coatings and drug depots formed from anti-inflammatory steroids have anti-inflammatory activity from the drugs being released from the prodrug shaped form.

In some embodiments, the surface coatings or drug depots provided herein are designed for the controlled and sustained release of a steroid drug from the prodrug dimer used to coat the article or in combination with an implantable medical device, respectively. The release rate from a surface coating or drug depot of the disclosure can be controlled through several engineerable design parameters, including: 1) selection of the steroid drug; 2) selection of the functional group of the drug for conjugation (e.g., if multiple exist); 3) selection of the linker; 4) selection of the linkage group (e.g., esters, carbonates, carbonate esters, or anhydrides); 5) selection of the surface area of the surface coatings or drug depots; and 6) selection of the drug loading in the surface coatings or drug depots (e.g., by adding traditional pharmaceutical excipients or mixing other steroid dimers as excipients when making the surface coatings or drug depots). Provided in certain instances herein is a coating and/or drug depot that comprises two or more radicals of a drug. Provided in certain instances herein is a coating and/or drug depot that comprises two or more steroid radicals (e.g., taken together with a linker to form a dimer provided herein). In some embodiments, the coating and/or drug depot comprising the two or more steroid radicals have a controlled release (e.g., as the free form of the two or more steroid radical) from the coating and/or drug depot. In some embodiments, the two or more steroid radicals come together to form a heterodimer (e.g., different steroid drugs on the two ends of a linker provided herein). In some embodiments, the two or more steroids come together to form a homodimer (e.g., the same steroid drugs on the two ends of a linker provided herein). In some embodiments, the coatings and/or drug depots comprise, steroid heterodimers, steroid homodimers, or a mixture thereof. Surface coatings or drug depots formed from the compounds provided herein can yield sustained and uniform release of the steroid compounds (e.g., without exhibiting any burst release (e.g., t₁₀ can be equal to or greater than 1/10 of t₅₀) and without reliance upon degradable matrices, which can cause undesirable local side effects (such as inflammation)). In some embodiments, the coatings and/or drug depots has a drug loading that is suitable for producing locally effective concentrations of a steroid drug for periods of days to weeks to months or even years.

Steroids can be used in combination with medical devices as combination products, or as adjunctive therapy, for a variety of medical fields including, for example, ophthalmology, oncology, laryngology, endocrinology and metabolic diseases, rheumatology, urology, neurology, cardiology, dental medicine, dermatology, otology, post-surgical medicine, orthopedics, pain management, and gynecology.

In certain embodiments, provided herein is a system comprising an article body and a steroid material. In some embodiments, the system is an article comprising an article body and a steroid material

Provided in some embodiments herein are surface coatings formed from compounds of formula (A-VIII) and articles or drug depots formed from compounds of formula (A-VIII):

D1-L-D2 (A-VIII)

or a pharmaceutically acceptable salt thereof, wherein each of D1 and D2 is, independently, a radical formed from a steroid; and L is a linker covalently linking D1 to D2. Each of D1 and D2 can be, independently, selected from an anabolic steroid, an androgenic steroid, a progestin steroid, an estrogen steroid, a cancer treatment steroid, an antibiotic steroid, a glucocorticoid steroid, a benign steroid, or a corticosteroid. In some embodiments, D1 and/or D2 are a mineralocorticoid steroid. L can be covalently linked to D1 and to D2 via one or more ester, carbonate, carbonate ester, or anhydride linkages. Ester, carbonate, carbonate ester, or anhydride linkages formed from a functional group on D1 and D2 can be selected from, e.g., hydroxyl or carboxy. For example, L can include the radical —C(O)—(R^{A})—C(O)—, —C(O)—OC(O)—(R^{A})—C(O)O—C(O)—, or —O—(R^{A})—O—, where R^{A} is a radical of a polyol and includes at least one free hydroxyl group or R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O)_{q}CH₂CH₂—, —(CH₂CH₂CH₂CH₂O)ᵣCH₂CH₂CH₂CH₂—, or —(CH₂CH(CH₃)O)ₛCH₂CH(CH₃)—, and q, r, and s are integers from 1 to 10 (e.g., 1 to 10, 1 to 5, or 5 to 10). The articles or implantable medical devices to which the surface coatings or drug depots, respectively, are applied can be any article and/or medical device, or surface thereof, described herein.

In some embodiments, the compound has the structure of formula (A-II):

D1-O-L-O-D2 (A-II),

or a pharmaceutically acceptable salt thereof, wherein each of D1-O and D2-O is, independently, a radical formed from a steroid.

In some embodiments, each of D1-O and D2-O is, independently, described by any one of formulas (I-a) to (I-zzz):

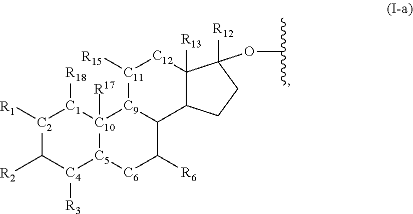

where the bond between C₁ and C₂, C₄ and C₅, C₅ and C₆, C₉ and C₁₀, and C₁₁ and C₁₂ is a single or a double bond; R₁ represents H, CH₃, or HC(O); R₂ represents ═O, OH, or H; or R₁ and R₂ taken together with carbons to which they are attached form an isoxazole; R₃ represents H, a halogen atom, or OH; R₆ represents H or CH₃; R₁₂ represents H, CH₃, or CH₃CH₂; R₁₃ represents CH₃ or CH₃CH₂; R₁₅ represents H or OH; R₁₇ represents H or CH₃; and R₁₈ represents H or CH₃;

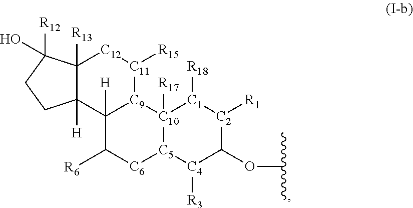

where the bond between C₁ and C₂, C₄ and C₅, C₅ and C₆, C₉ and C₁₀, and C₁₁ and C₁₂ is a single or a double bond; R₁ represents H, CH₃, or HC(O); R₃ represents H, a halogen atom, or OH; R₆ represents H or CH₃; R₁₂ represents H, CH₃, or CH₃CH₂; R₁₃ represents CH₃ or CH₃CH₂; R₁₅ represents H or OH; R₁₇ represents H or CH₃; and R₁₈ represents H or CH₃;

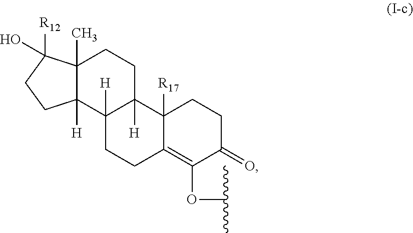

where R₁₂ represents H or CH₃; and R₁₇ represents H or CH₃;

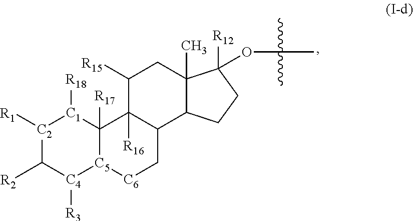

where the bond between C₁ and C₂, C₄ and C₅, and C₅ and C₆ is a single or a double bond; C₂ is O, C or CH₂; R₁ represents H, —CHOH, or is absent; R₂ represents ═O or OH; or R₁ and R₂ taken together with carbons to which they are attached form a pyrazole; R₃ represents H or OH; R₁₂ represents H, CH₃, optionally substituted alkynylene, C₁₋₆alkoxy, or CH₃CH₂; R₁₅ represents H or OH; R₁₆ represents H or a halogen atom; R₁₇ represents H or CH₃; and R₁₈ represents H or CH₃;

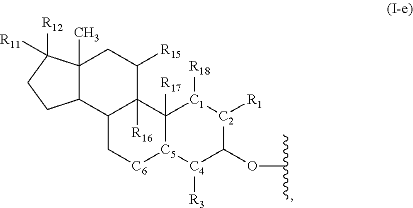

where the bond between C₁ and C₂, C₄ and C₅, and C₅ and C₆ is a single or a double bond; C₂ is O, C or CH₂; R₁ represents H, —CHOH, or is absent; R₃ represents H or OH; R₁₁ represents H, OH, CH₃, optionally substituted alkynylene, CH₃CH₂, ═O, —OC(O)CH₂CH₃, or is absent; R₁₂ represents H, OH, CH₃, optionally substituted alkynylene, CH₃CH₂, ═O, —OC(O)CH₂CH₃, or is absent; R₁₅ represents H or OH; R₁₆ represents H or a halogen atom; R₁₇ represents H or CH₃; and R₁₈ represents H or CH₃;

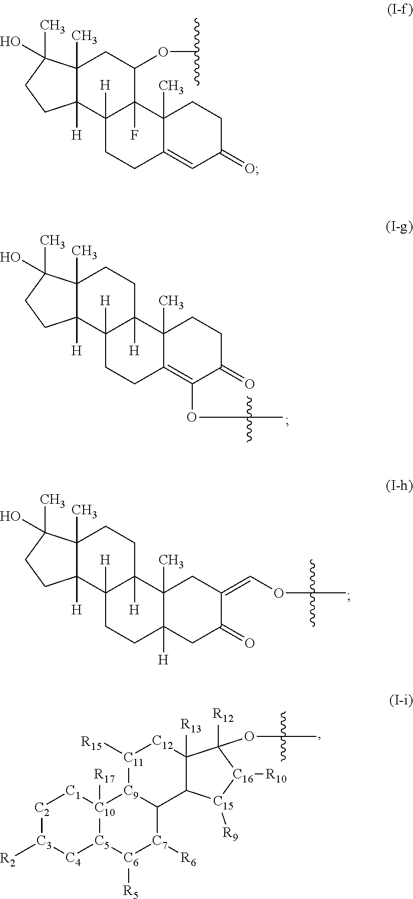

where the bond between C₁ and C₁₀, C₂ and C₃, C₃ and C₄, C₄ and C₅, C₅ and C₆, C₆ and C₇, C₈ and C₁₀, C₉ and C₁₀, C₁₁ and C₁₂, C₁₅ and C₁₆ is a single or a double bond; R₂ represents H, ═O, OH, —NOH, or C₁₋₆alkoxy; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R₁₂ represents H, optionally substituted alkynylene, —CH₂CH═CH₂, CH₃, —C(O)CH₃, or —CH═CH₂; R₁₃ represents CH₃ or CH₂CH₃; R₁₅ represents H or ═CH₂; and R₁₇ represents H, CH₃, or is absent;

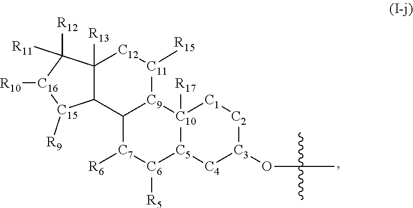

where the bond between C₁ and C₁₀, C₂ and C₃, C₄ and C₅, C₆ and C₇, C₅ and C₁₀, C₉ and C₁₀, C₁₁ and C₁₂, C₁₅ and C₁₆ is a single or a double bond; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R¹¹ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; R₁₂ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; or R₁₁ and R₁₂ together with carbon to which they are attached form a lactone; R₁₃ represents CH₃ or CH₂CH₃; R₁₅ represents H or ═CH₂; and R₁₇ represents H, CH₃, or is absent;

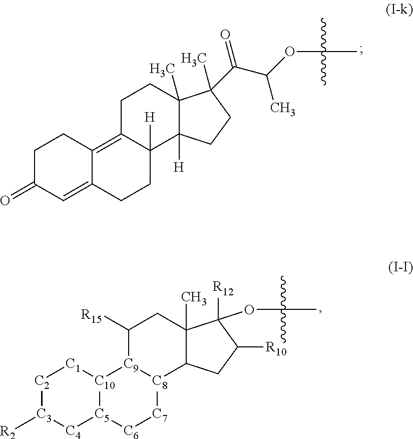

where the bond between C₁ and C₂, C₁ and C₁₀, C₂ and C₃, C₃ and C₄, C₄ and C₅, C₆ and C₇, C₅ and C₁₀, C₇ and C₈, and C₈ and C₉ is a single or a double bond; R₂ represents OH, —OC(O)Ph, or C₁₋₆alkoxy; R₁₀ represents H or OH; R₁₂ represents H, optionally substituted alkynylene; and R₁₅ represents H or C₁₋₆alkoxy;

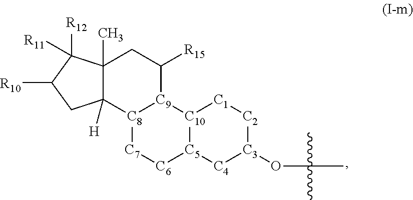

where the bond between C₁ and C₂, C₁ and C₁₀, C₂ and C₃, C₃ and C₄, C₄ and C₅, C₆ and C₇, C₅ and C₁₀, C₇ and C₈, and C₈ and C₉ is a single or a double bond; R₁₀ represents H or OH; R₁₁ represents H, OH, optionally substituted alkynylene, ═O, or is absent; R₁₂ represents H, OH, optionally substituted alkynylene, ═O, or is absent; and R₁₅ represents H or C₁₋₆alkoxy;

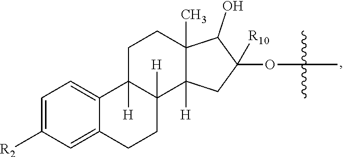

where R₂ represents OH or C₁₋₆alkoxy; and R₁₀ represents H or CH₃;

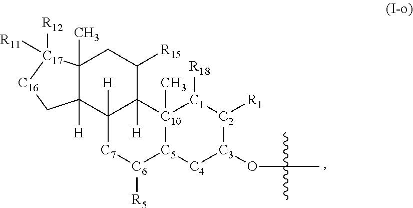

where the bond between C₁ and C₂, C₄ and C₅, C₅ and C₆, C₆ and C₇, and C₁₆ and C₁₇ is a single or a double bond; C₄ is NH, CH, or CH₂; R₁ represents H; R₅ represents H or a halogen atom; R₁₁ represents H, optionally substituted heteroaryl, —C(O)C₁₋₆ alkyl, —C(O)OC₁₋₆ alkyl, or —C(O)NHR, where R is optionally substituted alkyl or aryl; R₁₂ represents H, optionally substituted heteroaryl, —C(O)C₁₋₆ alkyl, —C(O)OC₁₋₆ alkyl, or —C(O)NHR, where R is optionally substituted alkyl or aryl; and R₁₈ represents H; or R₁ and R₁₈ taken together with carbons to which they are attached form a cyclopropane;

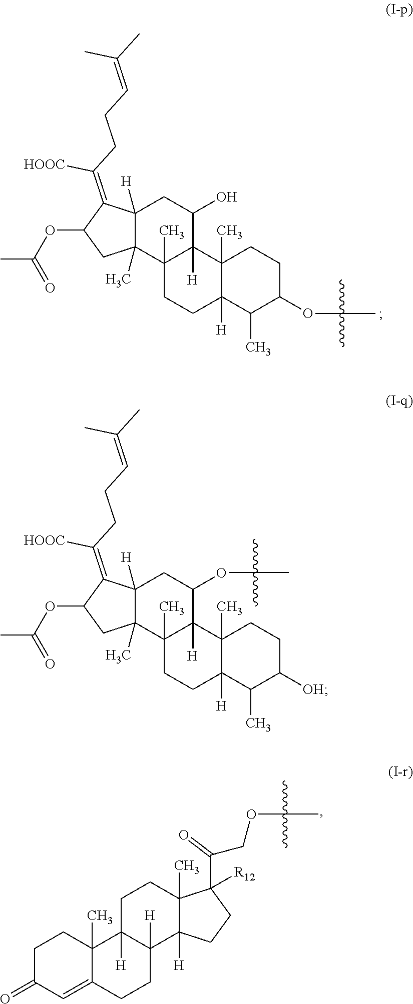

where R₁₂ is H or OH;

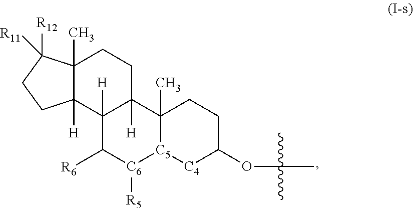

where the bond between C₄ and C₅, and C₅ and C₆ is a single or a double bond; R₅ represents H or C₁₋₆ alkyl; R₆ represents H or OH; R₁₁ represents H, OH, —C(O)C₁₋₆ alkyl, —C(O)CH₂OH, or —CH(CH₃)CH₂CH₂C(O)OH; and R₁₂ represents H, OH, —C(O)C₁₋₆ alkyl, —C(O)CH₂OH, or —CH(CH₃)CH₂CH₂C(O)OH;

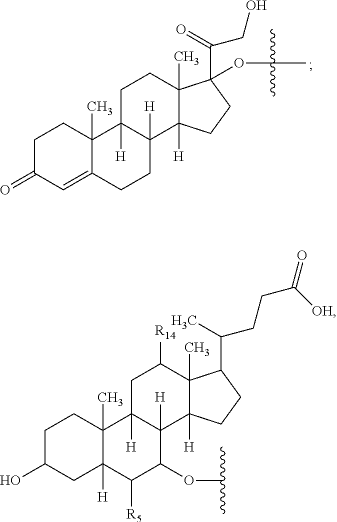

where R₅ represents H or CH₂CH₃; and R₁₄ represents H or OH;

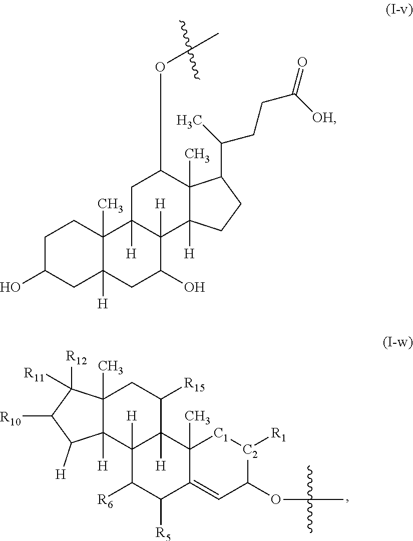

where the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₁ taken together with carbons to which they are attached form an optionally substituted cyclic acetal or optionally substituted heterocyclyl; R₁₂ represents H, OH, C₁₋₆ alkyl, optionally substituted —C(O)C₁₋₆ alkyl, —C(O)CH₂OC(O)C₁₋₆alkyl, optionally substituted —OC(O)C₁₋₆alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₂ taken together with carbons to which they are attached form an optionally substituted cyclic acetal or optionally substituted heterocyclyl; R₁₅ represents H, OH, ═O, or a halogen atom; and R₁₆ represents H or a halogen atom;

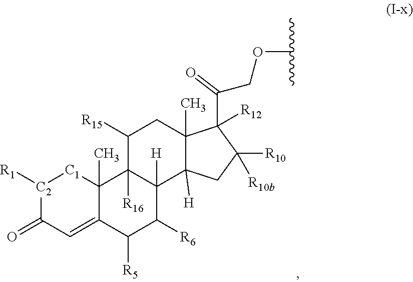

where the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆alkyl, OH, or ═CH₂; R_{10b} represents H, C₁₋₆alkyl, OH, ═CH₂, or be absent; R₁₂ represents H, OH, optionally substituted —C(O)C₁₋₆ alkyl, —C(O)CH₂OC(O)C₁₋₆ alkyl, optionally substituted —OC(O)C₁₋₆alkyl, or —OC(O)Ph; or R₁₀ and R₁₁ taken together with carbons to which they are attached form an optionally substituted cyclic acetal or optionally substituted heterocyclyl; R₁₅ represents H, OH, ═O, or a halogen atom; and R₁₆ represents H or a halogen atom;

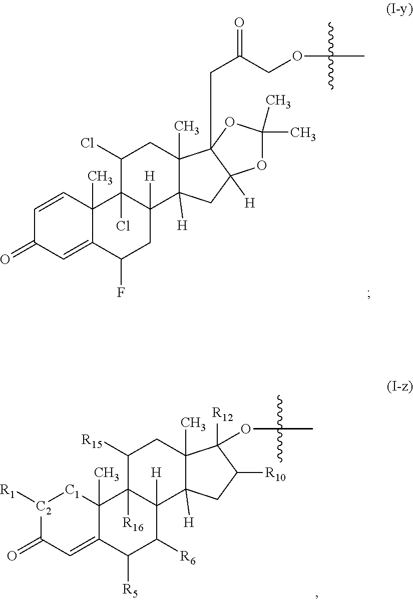

where the bond between C₁ and C₂ is a single or a double bond; R₁ represents H or a halogen atom; R₅ represents H, a halogen atom, or CH₃; R₆ represents H, a halogen atom; R₁₀ represents H, OH, CH₃, or ═CH₂; R₁₂ represents optionally substituted —C(O)C₁₋₆ alkyl, —C(O)CH₂OC(O)C₁₋₆ alkyl, or —C(O)SCH₂F; R₁₅ represents OH or ═O; and R₁₆ represents H or a halogen atom;

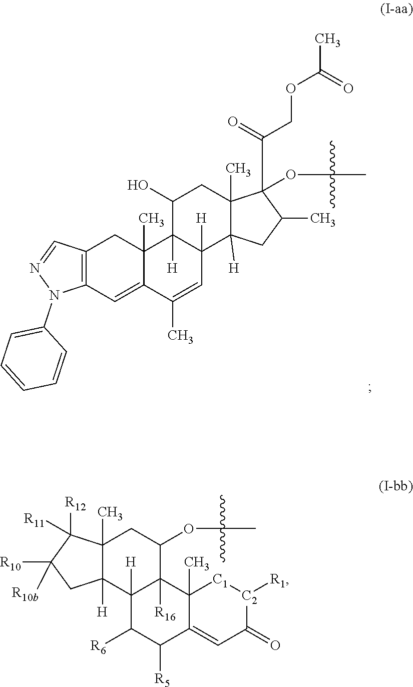

where the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆alkyl, OH, or ═CH₂; R_{10b} represents H, C₁₋₆alkyl, OH, or ═CH₂, or is absent; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₁ taken together with carbons to which they are attached form an optionally substituted cyclic acetal or optionally substituted heterocyclyl; R₁₂ represents H, OH, C₁₋₆alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆alkyl; or R₁₀ and R₁₂ taken together with carbons to which they are attached form an optionally substituted cyclic acetal or optionally substituted heterocyclyl; and R₁₆ represents H or a halogen atom;

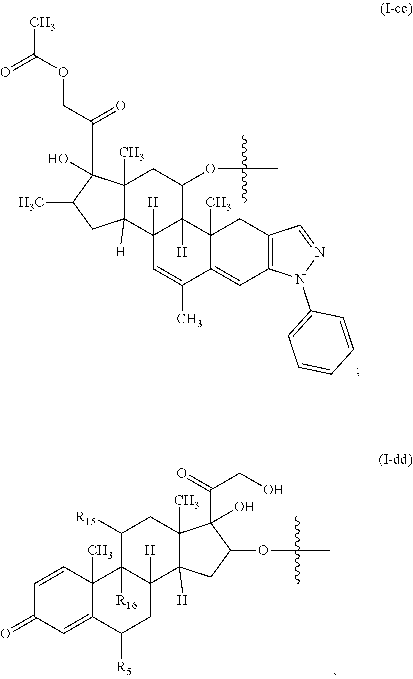

where R₅ represents H or a halogen atom; R₁₅ represents a halogen atom or OH; and R₁₆ represents H or a halogen atom;

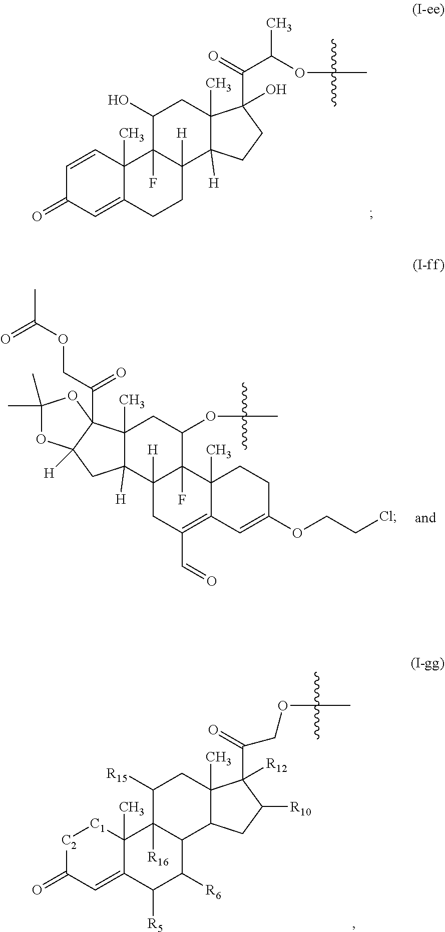

where the bond between C₁ and C₂ is a double or a single bond; R₁₆ represents H or a halogen atom; R₅ represents H, CH₃, or a halogen atom; R₁₂ represents H or a halogen atom; R₁₅ represents ═O or OH; R₁₂ and R₁₀ each, independently, represent —H, C₁₋₁₀ alkyl, —OH, —O-acyl, or R₁₂ and R₁₀ combine to form a cyclic acetal of formula (XVIII-a) where:

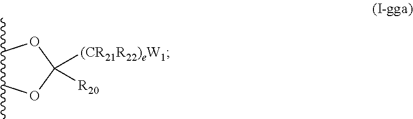

e is an integer from 0 to 6; R₂₀, R₂₁, and R₂₂ each, independently, represent H or C₁₋₁₀ alkyl; and W₁ represents H or CH₃;

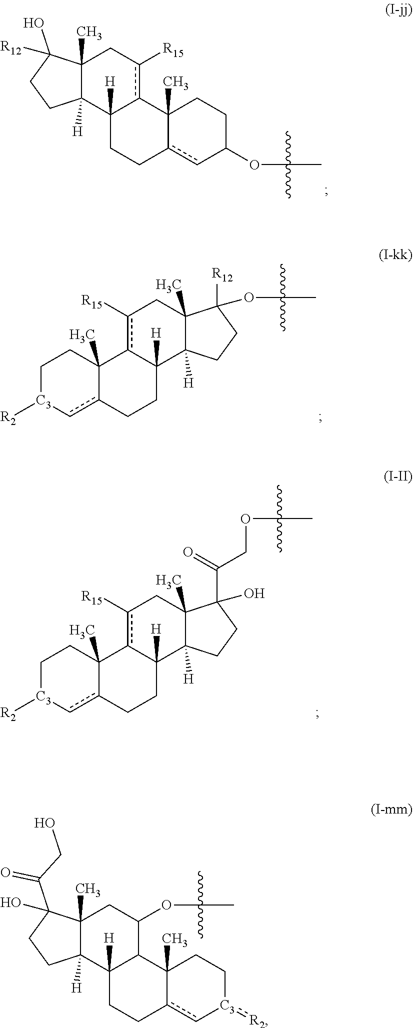

where the bond between C₃ and R₂ is a single or a double bond; R₂ represents OH or ═O; R₁₂ represents —C(═O)CH₂OC(═O)CH₃, —C(═O)CH₂OH, or —C(═O)CH₃; R₁₅ represents H or OH;

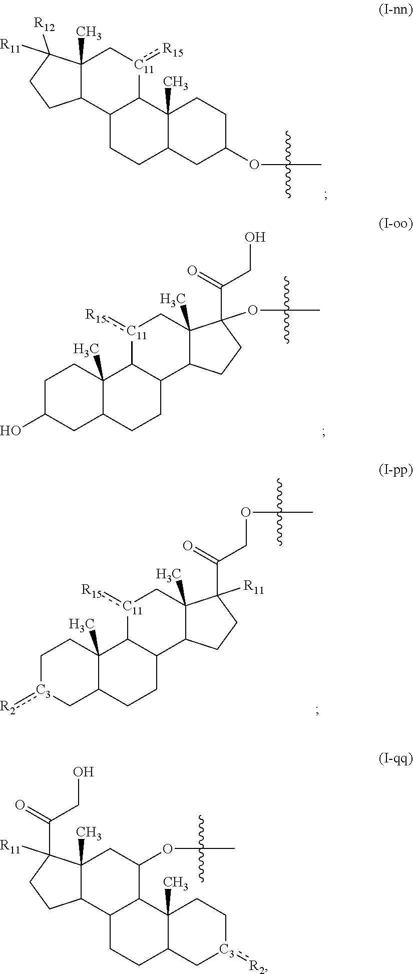

where the bond between C₃ and R₂, and C₁₁ and R₁₅ is a single or a double bond; R₂ represents OH or ═O; R₁₁ represents H, OH, —C(═O)CH₂OH, or —C(═O)CH₃; R₁₂ represents H, OH, —C(═O)CH₂OH, or —C(═O)CH₃; R₁₅ represents H, ═O, or OH;

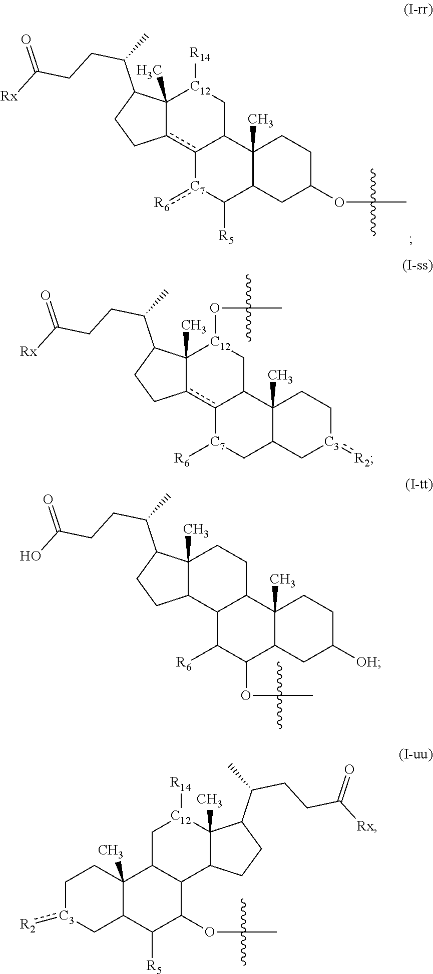

where the bond between C₃ and R₂, C₇ and R₆, and C₁₂ and R₁₄ is a single or a double bond; Rx represents OH, —NHCH₂C(═O)OH, or —NHCH₂CH₂SO₂OH; R₂ represents OH or ═O; R₅ represents H or OH; R₆ represents H, ═O, or OH; R₁₄ represents H, ═O, or OH;

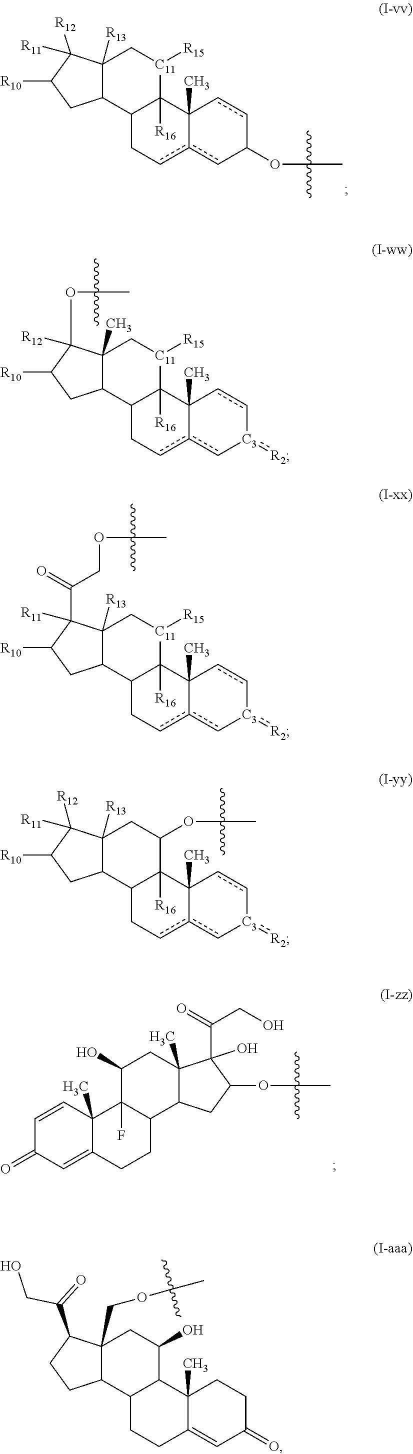

where the bond between C₃ and R₂, and C₁₁ and R₁₅ is a single or a double bond; R₂ represents OH or ═O; R₁₀ represents H or OH; R₁₁ represents H, OH, —C(═O)CH₂OH, —C(═O)OH, —C(═O)CH₂OH, or —C(═O)CH₃; R₁₂ represents H, OH, —C(═O)CH₂OH, —C(═O)OH, —C(═O)CH₂OH, or —C(═O)CH₃; R₁₃ represents —CH₂OH or —CH₃; R₁₅ represents H, OH, or ═O; R₁₆ represents H or F;

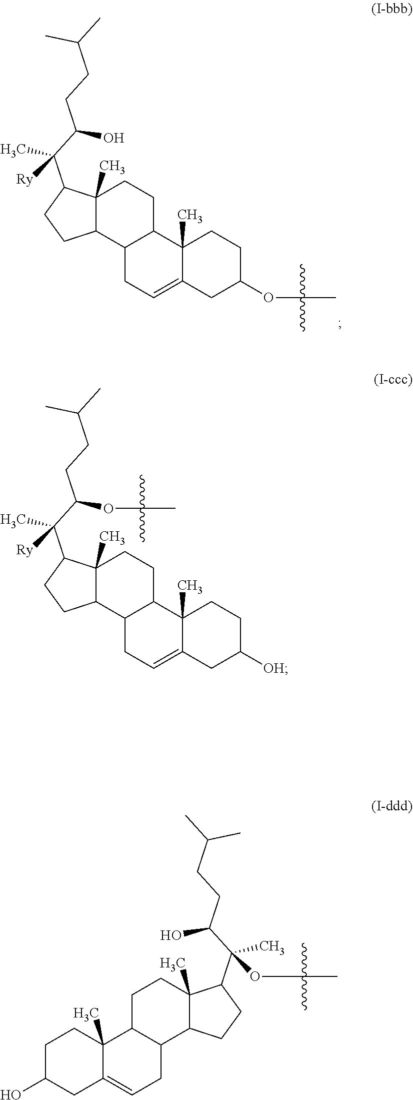

where Ry represents H or OH;

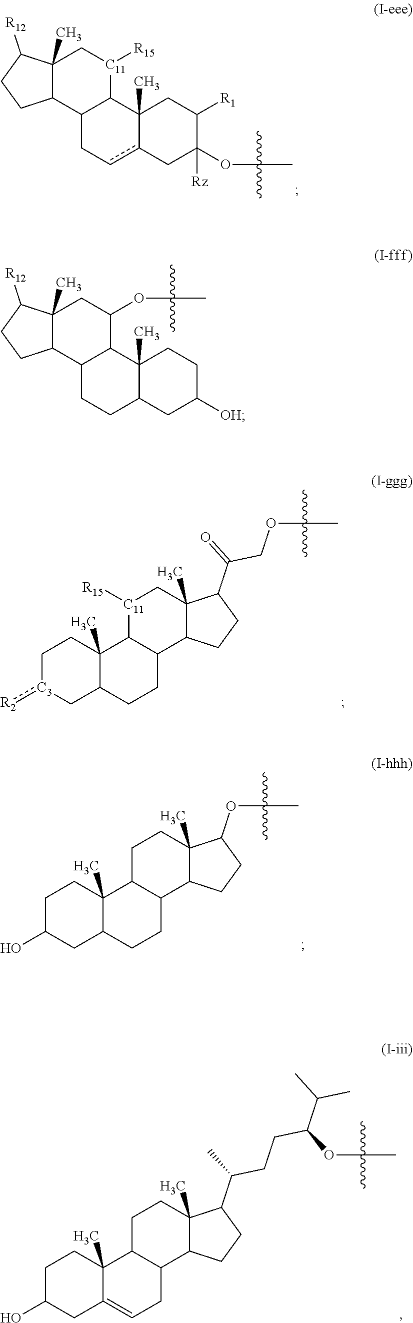

where the bond between C₃ and R₂, and C₁₁ and R₁₅ is a single or a double bond; Rz represents H or —CH₃; R₁ represents H or —OCH₂CH₃; R₂ represents OH or ═O; R₁₂ represents —OH, —C(═O)CH₃, —C(═O)CH₂OH, or —CH(CH₃)(CH₂)₂CH(OH)CH(CH₃)₂; R₁₅ represents H, —N(CH₃)₂, or ═O;

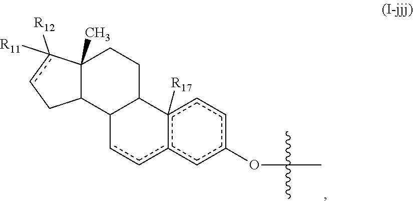

where the bond between C₃ and R₂ is a single or a double bond; R₂ represents OH or ═O; R₁₁ represents H, —C(═O)CH₃, —OC(═O)(CH₂)₄CH₃, or is absent; R₁₂ represents H, —C(═O)CH₃, —OC(═O)(CH₂)₄CH₃, or is absent; R₁₇ represents CH₃ or is absent;

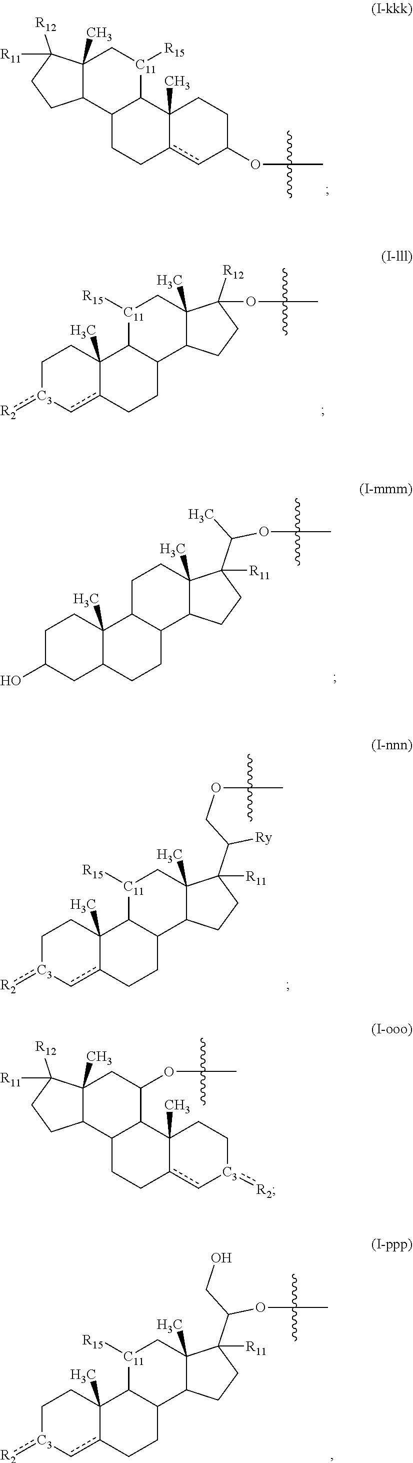

where the bond between C₃ and R₂, and C₁₁ and R₁₅ is a single or a double bond; Ry represents OH or ═O; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; R₁₅ represents H, ═O, or OH;

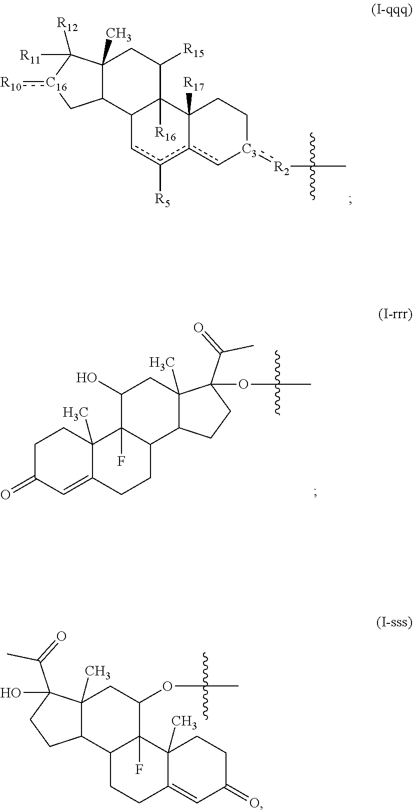

where the bond between C₃ and R₂, and C₁ and R₁₀ is a single or a double bond; R₂ represents OH or ═O; R₅ represents H, Cl, or —CH₃; R₁₀ represents H or ═CH₂; R₁₁ represents H, OH, —CH₃, —C(═O)CH₃, —C(═O)CH₂OC(═O)CH₃, or —OC(═O)CH₃; R₁₂ represents H, OH, —CH₃, —C(═O)CH₃, —C(═O)CH₂OC(═O)CH₃, or —OC(═O)CH₃; R₁₅ represents H or OH; R₁₆ represents F or H; R₁₇ represents H or —CH₃; or

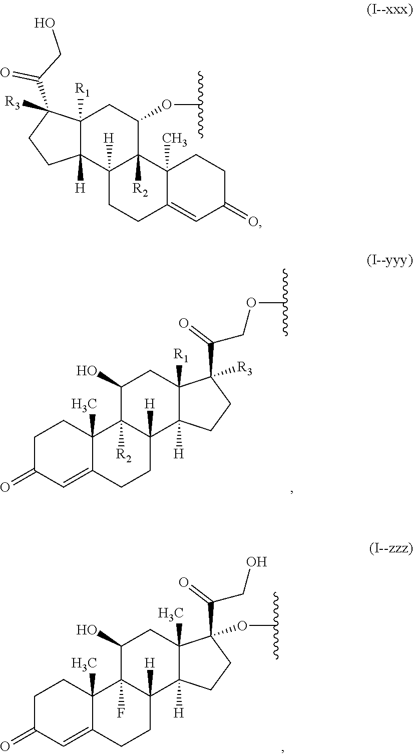

wherein R₁ is C(O)H or CH₃; R₂ represents H or F; R₃ represents H or OH.

In certain embodiments, the compound has the structure of formula (A-VII):

D1-C(O)-L-C(O)-D2 (A-VII),

or a pharmaceutically acceptable salt thereof, wherein each of D1-C(O) and D2-C(O) is, independently, a radical formed from a steroid; L is —O—C(O)—O—(R^{A})—O—C(O)—O—; and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. Each of D1-C(O) and D2-C(O) can, independently, be formed, for example, from fusidic acid, cholic acid, chenodeoxycholic acid, ursodeoxycholic acid, or obeticholic acid. In the drug dimers of formula (A-VII), D1-C(O)— and D2-C(O)— can further be described, for example, by formulas (I-hh), (I-ii), (I-ttt), (I-uuu), and (I-vvv) below.

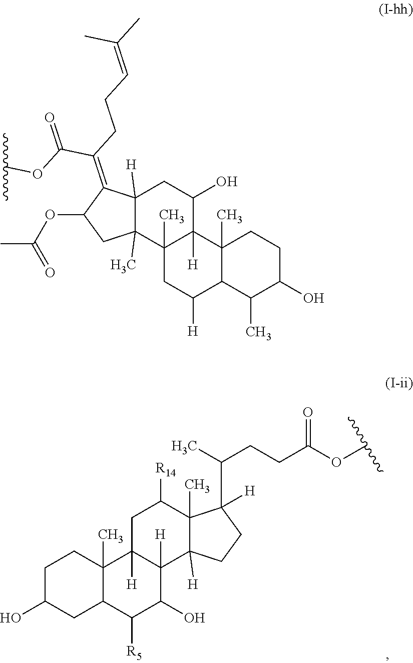

where R₅ represents H or C₁₋₆alkyl, R₁₄ represents H or OH;

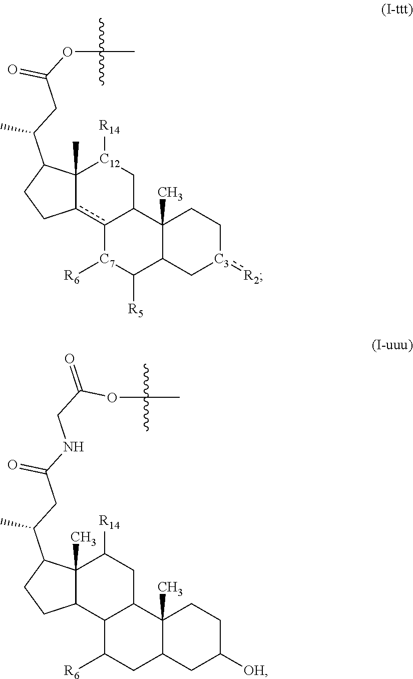

where the bond between C₃ and R₂, C₇ and R₆, and C₁₂ and R₁₄ is a single or a double bond; R₂ represents OH or ═O; R₅ represents H or OH; R₆ represents H, ═O, or OH; R₁₄ represents H, ═O, or OH;

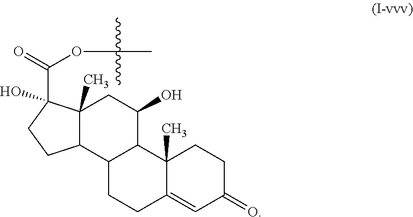 [SMILES: *OC(=O)[C@]1(O)CCC2C3CCC4=CC(=O)CC[C@@]4(C)C3[C@H](O)C[C@]21C]

In some embodiments, drug dimers provided herein are homodimers and heterodimers. In some embodiments, the drug dimers comprise a steroid, including, for example, anabolic steroids, and androgenic steroids, progestin steroids, estrogen steroids, cancer treatment steroids, antibiotic steroids, glucocorticoid steroids, benign steroids, corticosteroids, anti-angiogenic steroids, intraocular pressure (IOP) lowering steroids, cholic acid-related bile acid steroids, steroid metabolites, cholesterol-derivatives, neurosteroids, pheromones, progestins, or other steroids. Examples of anabolic steroids include, but are not limited to, androisoxazole, androstenediol, bolandiol, bolasterone, clostebol, ethylestrenol, formyldienolone, 4-hydroxy-19-nortestosterone, methandriol, methenolone, methyltrienolone, nandrolone, norbolethone, oxymesterone, stenbolone, and trenbolone. Androgenic steroids are, for example, boldenone, fluoxymesterone, mestanolone, mesterolone, methandrostenolone, 17-methyltestosterone, 17-α-methyltestosterone 3-cyclopentyl enol ether, norethandrolone, normethandrone, oxandrolone, oxymesterone, oxymetholone, prasterone, stanlolone, stanozolol, testosterone, testosterone 17-chloral hemiacetal, testosterone proprionate, testosterone enanthate tiomesterone dehydroepiandrosterone (DHEA), androstenedione, androstenediol, androsterone, dihydrotestosterone (DHT), androstanolone, and derivatives thereof. Exemplary progestin steroids are norethisterone, norethisterone acetate, gestodene, levonorgestrel, allylestrenol, anagestone, desogestrel, dimethisterone, dydrogesterone, ethisterone, ethynodiol, ethynodiol diacetate, etonogestrel, gestodene, ethinylestradiol, haloprogesterone, 17-hydroxy-16-methylene-progesterone, 17 alpha-hydroxyprogesterone, lynestrenol, medroxyprogesterone, melengestrol, norethindrone, norethynodrel, norgesterone, gestonorone, norethisterone, norgestimate, norgestrel, levonorgestrel, norgestrienone, norvinisterone, pentagestrone, MENT (7-methyl-19-testosterone); norelgestromin, and trimigestone drospirenone, tibolone, megestrol, and derivatives thereof. Examples of estrogen steroid are estrogen, eguilenin, equilin, 17β-estradiol, estradiol benzoate, estriol, ethinyl estradiol, mestranol, moxestrol, mytatrienediol, quinestradiol, and quinestrol. Steroids used in cancer treatment are, for example, abiraterone, cyproterone acetate, dutasteride, enzalutamide, finasteride, and galeterone. Exemplary antibiotic steroid is fusidic acid. Glucocorticoids include, for example, medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, loprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6α-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol. Exemplary benign steroids are cholesterol, 11-deoxycortisol, 11-deoxycorticosterone, pregnenolone, cholic acid, chenodeoxycholic acid, ursodeoxycholic acid, obeticholic acid, tetrahydrocortisone, tetrahydrodeoxycortisol, tetrahydrocorticosterone, 5α-dihydrocorticosterone, and 5α-dihydropregesterone. Exemplary anti-angiogenic steroids or intraocular pressure (IOP) lowering steroids are anecortave acetate, anecortave, 11-epicortisol, 17α-hydroxyprogesterone, tetrahydrocortexolone, and tetrahydrocortisol. Exemplary cholic acid-related bile acid steroids are deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, and tauroursodeoxycholic acid. Exemplary mineralocorticoid steroids are fludrocortisone and aldocortisone. Exemplary neurosteroids are alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, and 24(S)-hydroxycholesterol. Exemplary other steroids are flugestone, prebediolone, chlormadinone acetate, medrogestone, and segesterone acetate. Exemplary pheromones are androstadienol, androstadienone, androstenol, androstenone, estratetraenol, 5-dehydroprogesterone, 6-dehydro-retroprogesterone, allopregnanolone, and hydroxyprogesterone caproate. Exemplary steroid metabolites are tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, and progesterone. Exemplary progestins are allopregnone-3α,20α-diol, allopregnone-3β,20β-diol, allopregnane-3β,21-diol-11,20-dione, allopregnane-3β,17α-diol-20-one, 3,20-allopregnanedione,3β,11β,17α,20β,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,20β-triol, allopregnane-3β,17α,21-triol-11,20-dione, allopregnane-3β,11β,21-triol-20-one, allopregnane-3β,17α,21-triol-20-one, allopregnane-3α-ol-20-one, allopregnane-3β-ol-20-one, pregnanediol, 3,20-pregnanedione, 4-pregnene-20,21-diol-3,11-dione, 4-pregnene-11β,17α,20β,21-tetrol-3-one, 4-pregnene-17α,20β,21-triol-3,11-dione, 4-pregnene-17α,20β,21-triol-3-one, and pregnenolone.

The drug dimers useful in making the articles or drug depots of the disclosure can have any of formulas (A-I)-(LXXVIII), described herein.

### Steroid Homodimers

Provided in some embodiments herein are homodimers (e.g., drug depots or surface coatings formed from homodimers) of the formula (I):

D1-L-D2 (A-VIII)

or a pharmaceutically acceptable salt thereof, wherein D1 and D2 are radicals formed from the same steroid. L can be covalently linked to D1 and to D2 via one or more ester, carbonate, carbonate ester, or anhydride linkages. Ester, carbonate, carbonate ester, or anhydride linkages formed from a functional group on D1 and D2 can be selected from, e.g., hydroxyl or carboxy. For example, L can include the radical —C(O)—(R^{A})—C(O)—, —C(O)—OC(O)—(R^{A})—C(O)O—C(O)— or —O—(R^{A})—O—, where R^{A} is a radical of a polyol and includes at least one free hydroxyl group or R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, —(CH₂CH₂O)_{q}CH₂CH₂—, —(CH₂CH₂CH₂CH₂O)ᵣCH₂CH₂CH₂CH₂—, or —(CH₂CH(CH₃)O)ₛCH₂CH(CH₃)—, and q, r, and s are integers from 1 to 10 (e.g., 1 to 10, 1 to 5, or 5 to 10). The homodimer can be further described by one of formulas (II)-(LXXVIII), below.

In some embodiments, the steroid is an anabolic steroid and the drug dimer is further described by the formula (II):

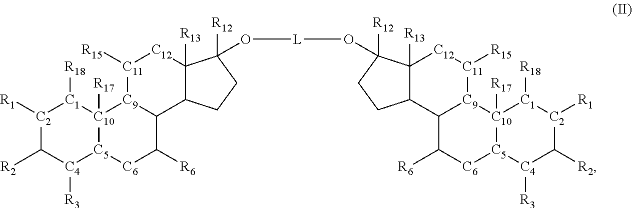

wherein the bond between C₁ and C₂, C₄ and C₅, C₅ and C₆, C₉ and C₁₀, and C₁₁ and C₁₂ is a single or a double bond; R₁ represents H, CH₃, or HC(O); R₂ represents ═O, OH, or H; or R₁ and R₂ taken together with carbons to which they are attached form an isoxazole; R₃ represents H, a halogen atom, or OH; R₆ represents H or CH₃; R₁₂ represents H, CH₃, or CH₃CH₂; R₁₃ represents CH₃ or CH₃CH₂; R₁₅ represents H or OH; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (II) can be formed from an anabolic steroid selected from the group consisting of androisoxazole, androstenediol, bolandiol, bolasterone, clostebol, ethylestrenol, formyldienolone, 4-hydroxy-19-nortestosterone, methandriol, methenolone, methyltrienolone, nandrolone, norbolethone, oxymesterone, stenbolone, and trenbolone.

In certain embodiments, the steroid is an anabolic steroid and the drug dimer is further described by the formula (III):

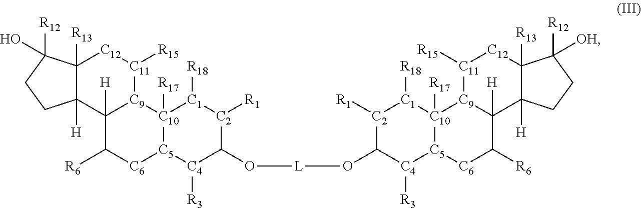

wherein the bond between C₁ and C₂, C₄ and C₅, C₅ and C₆, C₉ and C₁₀, and C₁₁ and C₁₂ is a single or a double bond; R₁ represents H, CH₃, or HC(O); R₃ represents H, a halogen atom, or OH; R₆ represents H or CH₃; R₁₂ represents H, CH₃, or CH₃CH₂; R₁₃ represents CH₃ or CH₃CH₂; R₁₅ represents H or OH; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (III) can be formed from an anabolic steroid selected from the group consisting of androstenediol, bolandiol, bolasterone, clostebol, formyldienolone, 4-hydroxy-19-nortestosterone, methandriol, methenolone, methyltrienolone, nandrolone, norbolethone, oxymesterone, stenbolone, and trenbolone.

In some embodiments, the steroid is an anabolic steroid and the drug dimer is further described by the formula (IV):

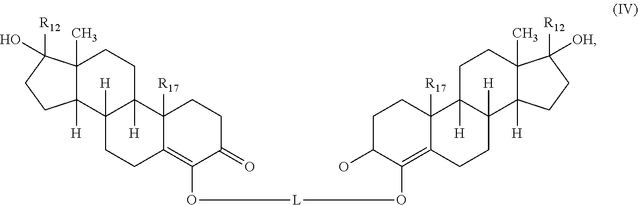

wherein R₁₂ represents H or CH₃; R₁₇ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (IV) can be formed from an anabolic steroid selected from 4-hydroxy-19-nortestosterone or oxymesterone.

In certain embodiments, the steroid is an androgenic steroid and the drug dimer is further described by the formula (V):

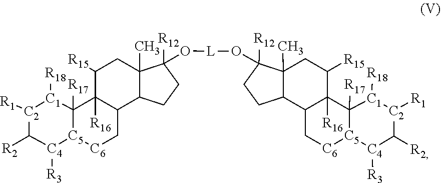

wherein the bond between C₁ and C₂, C₄ and C₅, and C₅ and C₆ is a single or a double bond; C₂ is O, C or CH₂; R₁ represents H, —CHOH, or is absent; R₂ represents ═O or OH; or R₁ and R₂ taken together with carbons to which they are attached form a pyrazole; R₃ represents H or OH; R₁₂ represents H, CH₃, optionally substituted alkynylene, C₁₋₆alkoxy, or CH₃CH₂; R₁₅ represents H or OH; R₁₆ represents H or a halogen atom; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (V) can be formed from an androgenic steroid selected from the group consisting of boldenone, fluoxymesterone, mestanolone, mesterolone, methandrostenolone, 17-methyltestosterone, 17-α-methyltestosterone 3-cyclopentyl enol ether, norethandrolone, normethandrone, oxandrolone, oxymesterone, oxymetholone, prasterone, stanlolone, stanozolol, testosterone, testosterone enanthate tiomesterone dehydroepiandrosterone (DHEA), androstenedione, androstenediol, androsterone, and dihydrotestosterone (DHT).

In some embodiments, the steroid is an androgenic steroid and the drug dimer is further described by the formula (VI):

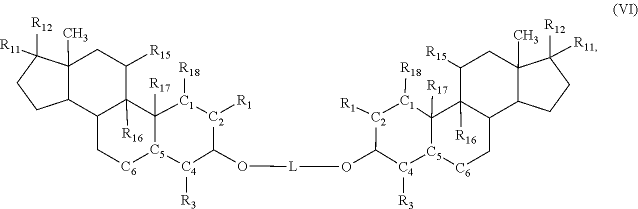

wherein the bond between C₁ and C₂, C₄ and C₅, and C₅ and C₆ is a single or a double bond; C₂ is O, C or CH₂; R₁ represents H, —CHOH, or is absent; R₃ represents H or OH; R₁₁ represents H, OH, CH₃, optionally substituted alkynylene, CH₃CH₂, ═O, —OC(O)CH₂CH₃, or is absent; R₁₂ represents H, OH, CH₃, optionally substituted alkynylene, CH₃CH₂, ═O, —OC(O)CH₂CH₃, or is absent; R₁₅ represents H or OH; R₁₆ represents H or a halogen atom; R₁₇ represents H or CH₃; R₁₈ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (VI) can be formed from an androgenic steroid selected from the group consisting of boldenone, fluoxymesterone, mestanolone, mesterolone, methandrostenolone, 17-methyltestosterone, norethandrolone, normethandrone, oxandrolone, oxymesterone, oxymetholone, prasterone, stanlolone, testosterone, testosterone proprionate, testosterone enanthate tiomesterone dehydroepiandrosterone (DHEA), androstenedione, androstenediol, androsterone, and dihydrotestosterone (DHT).

In certain embodiments, the steroid is an androgenic steroid and the drug dimer is further described by the formula (VII):

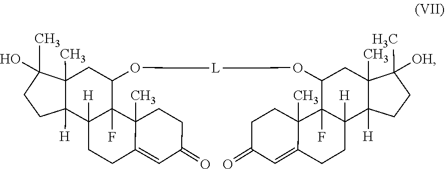

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (VII) can be formed from the androgenic steroid fluoxymesterone.

In some embodiments, the steroid is an androgenic steroid and the drug dimer is further described by the formula (VIII):

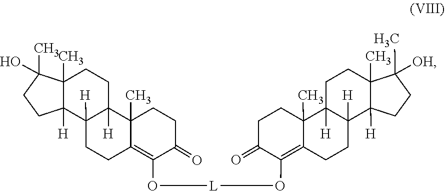

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (VIII) can be formed from the androgenic steroid oxymesterone.

In some embodiments, the steroid is an androgenic steroid and the drug dimer is further described by the formula (IX):

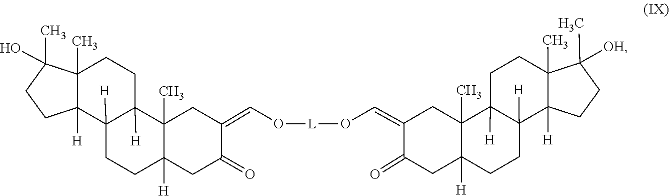

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (IX) can be formed from the androgenic steroid oxymetholone.

In particular embodiments, the steroid is a progestin steroid and the drug dimer is further described by the formula (X):

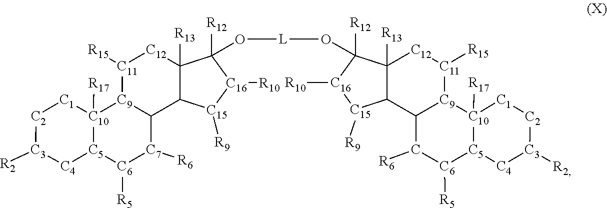

wherein the bond between C₁ and C₁₀, C₂ and C₃, C₃ and C₄, C₄ and C₅, C₅ and C₆, C₆ and C₇, C₅ and C₁₀, C₉ and C₁₀, C₁₁ and C₁₂, C₁₅ and C₁₆ is a single or a double bond; R₂ represents H, ═O, OH, —NOH, or C₁₋₆alkoxy; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R₁₂ represents H, optionally substituted alkynylene, —CH₂CH═CH₂, CH₃, —C(O)CH₃, or —CH═CH₂; R₁₃ represents CH₃ or CH₂CH₃; R₁₅ represents H or ═CH₂; R₁₇ represents H, CH₃, or is absent; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (X) can be formed from a progestin steroid selected from the group consisting of norethisterone, gestodene, levonorgestrel, allylestrenol, anagestone, desogestrel, dimethisterone, dydrogesterone, ethisterone, ethynodiol, etonogestrel, gestodene, ethinylestradiol, 17-hydroxy-16-methylene-progesterone, 17 alpha-hydroxyprogesterone, lynestrenol, medroxyprogesterone, melengestrol, norethindrone, norethynodrel, norgesterone, gestonorone, norethisterone, norgestrel, levonorgestrel, norgestrienone, pentagestrone, 7-methyl-19-testosterone (MENT), norelgestromin, tibolone, and megestrol.

In certain embodiments, the steroid is a progestin steroid and the drug dimer is further described by the formula (XI):

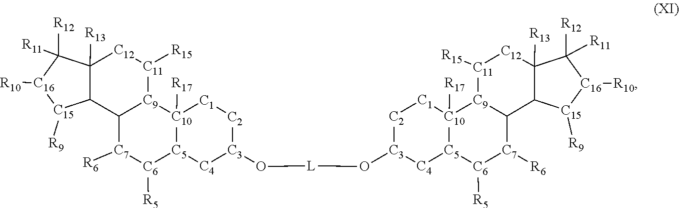

wherein the bond between C₁ and C₁₀, C₂ and C₃, C₄ and C₅, C₆ and C₇, C₅ and C₁₀, C₉ and C₁₀, C₁₁ and C₁₂, C₁₅ and C₁₆ is a single or a double bond; R₅ represents H, CH₃, or a halogen atom; R₆ represents H or CH₃; or R₅ and R₆ taken together with carbons to which they are attached form a cyclopropane; R₉ is H; R₁₀ is H or ═CH₂; or R₉ and R₁₀ taken together with carbons to which they are attached form a cyclopropane; R¹¹ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; R₁₂ represents H, OH, optionally substituted alkynylene, —C(O)CH₃, —CH₂CH═CH₂, a halogen atom, —CH═CH₂, —OC(O)CH₃, CH₃, —C(O)C(OH)CH₃; or R₁₁ and R₁₂ together with carbon to which they are attached form a lactone; R₁₃ represents CH₃ or CH₂CH₃; R₁₅ represents H or ═CH₂; R₁₇ represents H, CH₃, or is absent; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XI) can be formed from a progestin steroid selected from the group consisting of norethisterone, norethisterone acetate, gestodene, levonorgestrel, dimethisterone, dydrogesterone, ethisterone, ethynodiol, etonogestrel, gestodene, ethinylestradiol, haloprogesterone, 17-hydroxy-16-methylene-progesterone, 17 alpha-hydroxyprogesterone, medroxyprogesterone, melengestrol, norethindrone, norethynodrel, norgesterone, gestonorone, norethisterone, norgestimate, norgestrel, levonorgestrel, norgestrienone, 7-methyl-19-testosterone (MENT), norelgestromin, trimigestone, drospirenone, tibolone, and megestrol.

In some embodiments, the steroid is a progestin steroid and the drug dimer is further described by the formula (XII):

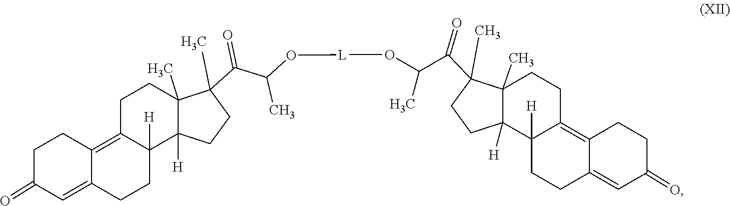

Wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XII) can be formed from the progestin steroid trimigestone.

In particular embodiments, the steroid is an estrogen steroid and the drug dimer is further described by the formula (XIII):

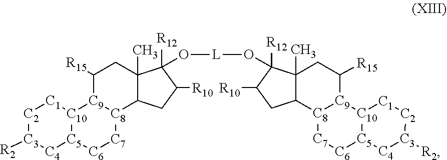

wherein the bond between C₁ and C₂, C₁ and C₁₀, C₂ and C₃, C₃ and C₄, C₄ and C₅, C₆ and C₇, C₅ and C₁₀, C₇ and C₈, and C₈ and C₉ is a single or a double bond; R₂ represents OH, —OC(O)Ph, or C₁₋₆alkoxy; R₁₀ represents H or OH; R₁₂ represents H, optionally substituted alkynylene; R₁₅ represents H or C₁₋₆ alkoxy; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XIII) can be formed from an estrogen steroid selected from the group consisting of estrogen, eguilenin, equilin, 17β-estradiol, estradiol benzoate, estriol, ethinyl estradiol, mestranol, moxestrol, mytatrienediol, quinestradiol, and quinestrol.

In some embodiments, the steroid is an estrogen steroid and the drug dimer is further described by the formula (XIV):

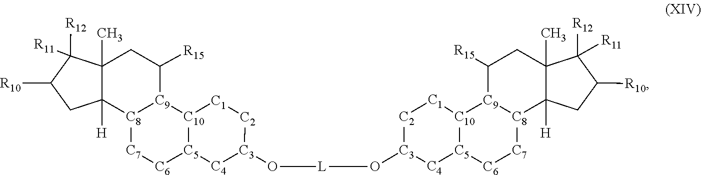

wherein the bond between C₁ and C₂, C₁ and C₁₀, C₂ and C₃, C₃ and C₄, C₄ and C₅, C₆ and C₇, C₅ and C₁₀, C₇ and C₈, and C₈ and C₉ is a single or a double bond; R₁₀ represents H or OH; R₁₁ represents H, OH, optionally substituted alkynylene, ═O, or is absent; R₁₂ represents H, OH, optionally substituted alkynylene, ═O, or is absent; R₁₅ represents H or C₁₋₆alkoxy; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XIV) can be formed from an estrogen steroid selected from the group consisting of estrogen, eguilenin, equilin, 17β-estradiol, estriol, ethinyl estradiol, and moxestrol.

In some embodiments, the steroid is an estrogen steroid and the drug dimer is further described by the formula (XV):

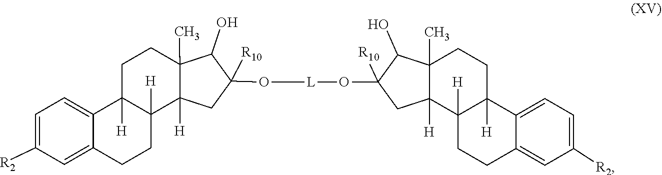

wherein R₂ represents OH or C₁₋₆ alkoxy; R₁₀ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XV) can be formed from an estrogen steroid selected from the group consisting of estriol, mytatrienediol, and quinestradiol.

In particular embodiments, the steroid is a cancer treatment steroid and the drug dimer is further described by the formula (XVI):

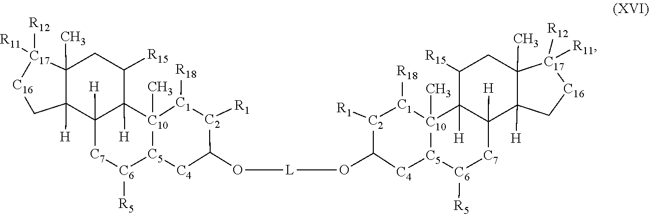

wherein the bond between C₁ and C₂, C₄ and C₅, C₅ and C₆, C₆ and C₇, and C₁₆ and C₁₇ is a single or a double bond; C₄ is NH, CH, or CH₂; R₁ represents H; R₅ represents H or a halogen atom; R₁₁ represents H, optionally substituted heteroaryl, —C(O)C₁₋₆ alkyl, —C(O)OC₁₋₆ alkyl, or —C(O)NHR, wherein R is optionally substituted alkyl or aryl; R₁₂ represents H, optionally substituted heteroaryl, —C(O)C₁₋₆ alkyl, —C(O)OC₁₋₆ alkyl, or —C(O)NHR, wherein R is optionally substituted alkyl or aryl; R₁₈ represents H; or R₁ and R₁₈ taken together with carbons to which they are attached form a cyclopropane; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XVI) can be formed from a cancer treatment steroid selected from the group consisting of abiraterone, cyproterone acetate, dutasteride, finasteride, and galeterone.

In some embodiments, the steroid is an antibiotic steroid and the drug dimer is further described by the formula (XVII):

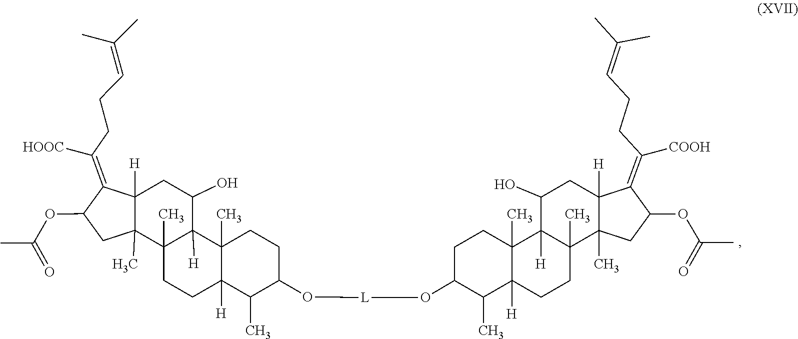

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XVII) can be formed from the steroid antibiotic fusidic acid.

In some embodiments, the steroid is an antibiotic steroid and the drug dimer is further described by the formula (XVIII):

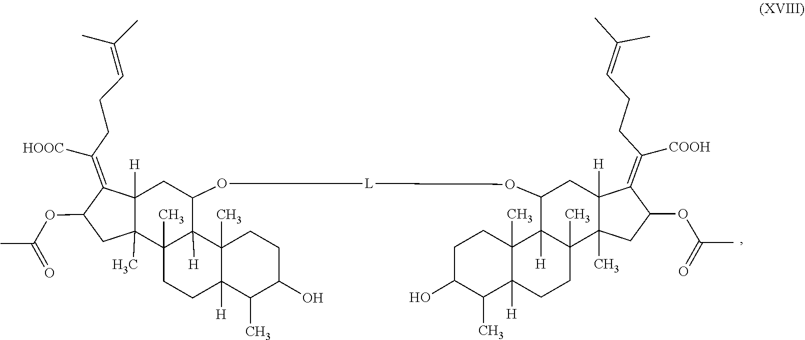

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XVIII) can be formed from the steroid antibiotic fusidic acid.

In some embodiments, the steroid is a benign steroid and the drug dimer is further described by the formula (XIX):

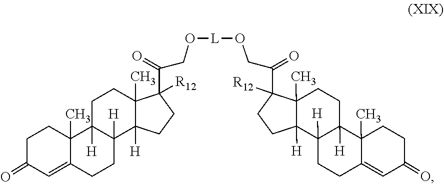

wherein R₁₂ is H or OH; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XIX) can be formed from a benign steroid selected from 11-deoxycortisol and 11-deoxycorticosterone.

In some embodiments, the steroid is a benign steroid and the drug dimer is further described by the formula (XX):

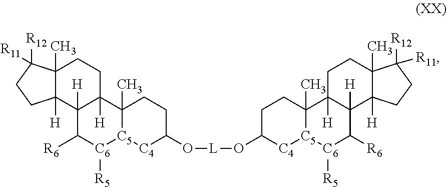

wherein the bond between C₄ and C₅, and C₅ and C₆ is a single or a double bond; R₅ represents H or C₁₋₆ alkyl; R₆ represents H or OH; R₁₁ represents H, OH, —C(O)C₁₋₆ alkyl, —C(O)CH₂OH, or —CH(CH₃)CH₂CH₂C(O)OH; R₁₂ represents H, OH, —C(O)C₁₋₆ alkyl, —C(O)CH₂OH, or —CH(CH₃)CH₂CH₂C(O)OH; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XX) can be formed from a benign steroid selected from the group consisting of cholesterol, 11-deoxycortisol, 11-deoxycorticosterone, pregnenolone, cholic acid, chenodeoxycholic acid, ursodeoxycholic acid, and obeticholic acid.

In some embodiments, the steroid is a benign steroid and the drug dimer is further described by the formula (XXI):

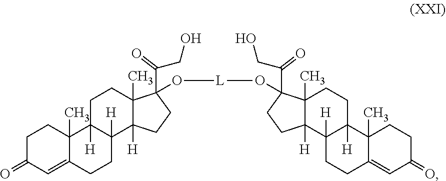

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms.
The drug dimer of formula (XXI) can be formed from a benign steroid including 11-deoxycortisol.

In some embodiments, the steroid is a benign steroid and the drug dimer is further described by the formula (XXII):

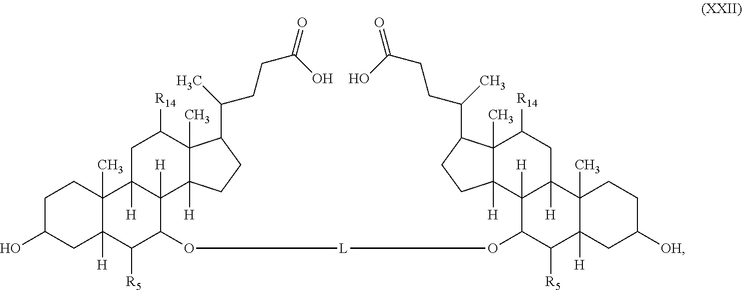

wherein R₅ represents H or CH₂CH₃; R₁₄ represents H or OH; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXII) can be formed from a benign steroid selected from the group consisting of cholic acid, chenodeoxycholic acid, ursodeoxycholic acid, and obeticholic acid.

In some embodiments, the steroid is a benign steroid and the drug dimer is further described by the formula (XXIII):

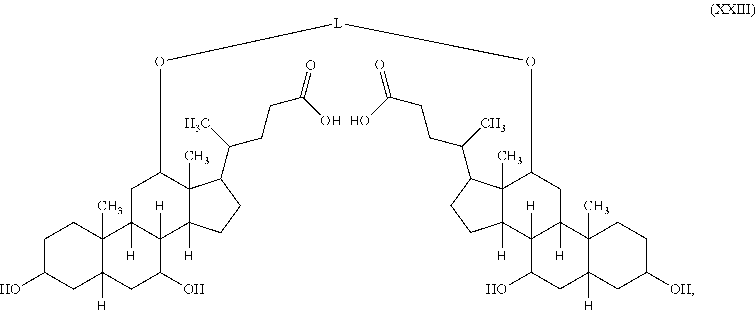

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXIII) can be formed from the benign steroid cholic acid.

In some embodiments, the steroid is a glucocorticoid steroid and the drug dimer is further described by the formula (XXIV):

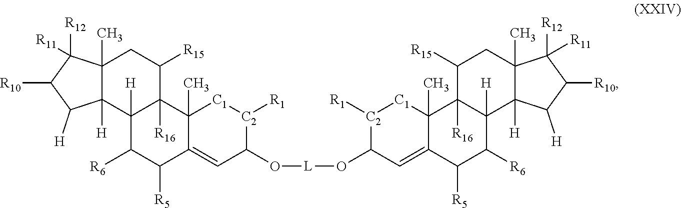

wherein the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆alkyl; or R₁₀ and R₁₁ taken together with carbons to which they are attached form an optionally substituted cyclic acetal or optionally substituted heterocyclyl; R₁₂ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₂ taken together with carbons to which they are attached form an optionally substituted cyclic acetal or optionally substituted heterocyclyl; R₁₅ represents H, OH, ═O, or a halogen atom; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXIV) can be formed from a glucocorticoid steroid selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cortisol, cortisone, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol.

In some embodiments, the steroid is a glucocorticoid steroid and the drug dimer is further described by the formula (XXV):

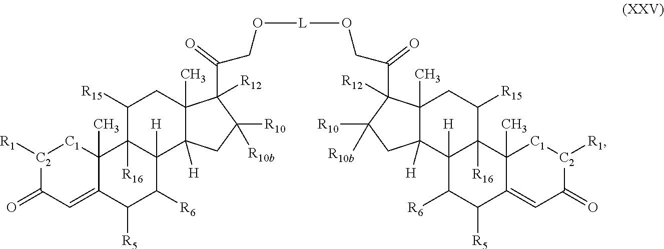

wherein the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆ alkyl, OH, or ═CH₂; R_{10b} represents H, C₁₋₆ alkyl, OH, ═CH₂, or be absent; R₁₂ represents H, OH, optionally substituted —C(O)C₁₋₆ alkyl, —C(O)CH₂OC(O)C₁₋₆ alkyl, optionally substituted —OC(O)C₁₋₆alkyl, or —OC(O)Ph; or R₁₀ and R₁₁ taken together with carbons to which they are attached form an optionally substituted cyclic acetal or optionally substituted heterocyclyl; R₁₅ represents H, OH, ═O, or a halogen atom; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXV) can be formed from a glucocorticoid steroid selected from the group consisting of alclometasone, beclometasone, betamethasone, betamethasone benzoate, betamethasone valerate, budesonide, cortisol, cortisone, desonide, desoximetasone, desoxycortone, desoxymethasone, dexamethasone, diflorasone, diflucortolone, difluorocortolone, fluclorolone, fluclorolone acetonide, fludroxycortide, flumetasone, flumethasone, flunisolide, flunisolide, fluocinolone, fluocinolone acetonide, fluocortolone, fluorocortisone, fluprednidene, fluprednisolone, halometasone, hydrocortisone, hydrocortisone butyrate, meprednisone, 6a-methylprednisolone, methylprednisolone, paramethasone, prednisolone, prednisone, prednylidene, triamcinolone, and triamcinolone acetonide.

In some embodiments, the steroid is a glucocorticoid steroid and the drug dimer is further described by the formula (XXVI):

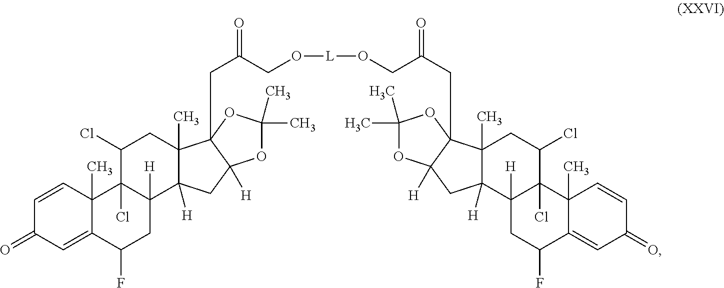

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXVI) can be formed from the glucocorticoid steroid fluclorolone acetonide.

In some embodiments, the steroid is a glucocorticoid steroid and the drug dimer is further described by the formula (XXVII):

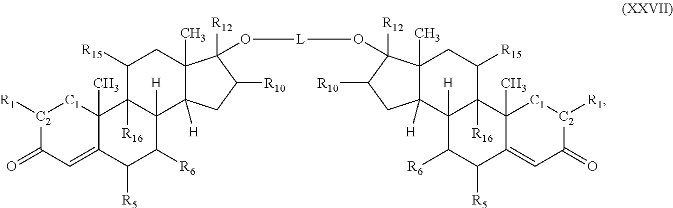

wherein the bond between C₁ and C₂ is a single or a double bond; R₁ represents H or a halogen atom; R₅ represents H, a halogen atom, or CH₃; R₆ represents H, a halogen atom; R₁₀ represents H, OH, CH₃, or ═CH₂; R₁₂ represents optionally substituted —C(O)C₁₋₆ alkyl, —C(O)CH₂OC(O)C₁₋₆alkyl, or —C(O)SCH₂F; R₁₅ represents OH or ═O; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXVII) can be formed from a glucocorticoid steroid selected from the group consisting of alclometasone, beclometasone, betamethasone, clobetasol, clobetasone, cortisol, cortisone, dexamethasone, diflorasone, fluclorolone, flumetasone, flumethasone, flumethasone pivalate, fluocinolone, fluorocortisone, fluorometholone, fluperolone, fluprednidene, fluprednidene acetate, fluprednisolone, fluticasone, halometasone, hydrocortisone, hydrocortisone acetate, hydrocortisone butyrate, meprednisone, 6α-methylprednisolone, methylprednisolone, methylprednisolone acetate, mometasone, paramethasone, prednisolone, prednisone, prednylidene, tixocortol, triamcinolone, and ulobetasol.

In certain embodiments, the steroid is a glucocorticoid steroid and the drug dimer is further described by the formula (XXVIII):

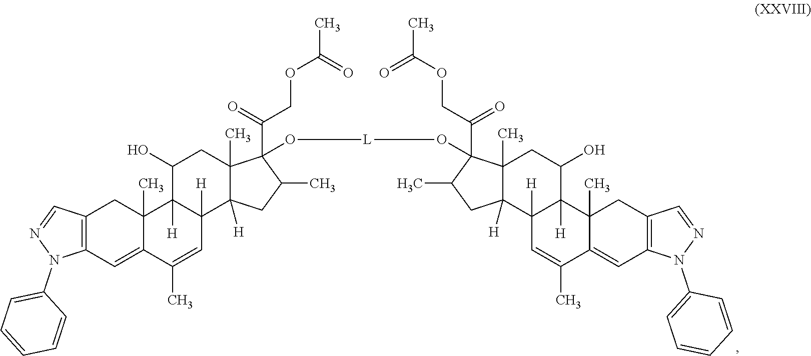

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXVIII) can be formed from the glucocorticoid steroid cortivazol.

In some embodiments, the steroid is a glucocorticoid steroid and the drug dimer is further described by the formula (XXIX):

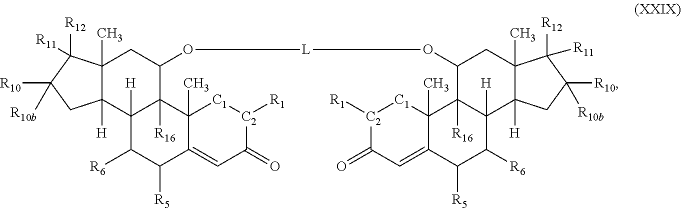

wherein the bond between C₁ and C₂ is a single or a double bond; R₁ represents H or a halogen atom; R₅ represents H, C₁₋₆ alkyl, or a halogen atom; R₆ represents H or a halogen atom; R₁₀ represents H, C₁₋₆alkyl, OH, or ═CH₂; R_{10b} represents H, C₁₋₆alkyl, OH, or ═CH₂, or is absent; R₁₁ represents H, OH, C₁₋₆ alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆ alkyl; or R₁₀ and R₁₁ taken together with carbons to which they are attached form an optionally substituted cyclic acetal or optionally substituted heterocyclyl; R₁₂ represents H, OH, C₁₋₆alkyl, optionally substituted —C(O)C₁₋₆ alkyl, —C(O)CH₂OC(O)C₁₋₆ alkyl, optionally substituted —OC(O)C₁₋₆ alkyl, —OC(O)Ph, —OC(O)heterocyclyl, —CH₂C(O)CH₂OH, —C(O)C(O)OH, —C(O)C(O)OC₁₋₆ alkyl, —C(O)SCH₂F, or —OC(O)OC₁₋₆alkyl; or R₁₀ and R₁₂ taken together with carbons to which they are attached form an optionally substituted cyclic acetal or optionally substituted heterocyclyl; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXIX) can be formed from a glucocorticoid steroid selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cortisol, cortisone, cortivazol, deflazacort, desonide, desoximetasone, desoxymethasone, dexamethasone, diflorasone, diflorasone diacetate, diflucortolone, diflucortolone valerate, difluorocortolone, difluprednate, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol.

In some embodiments, the steroid is a glucocorticoid steroid and the drug dimer is further described by the formula (XXX):

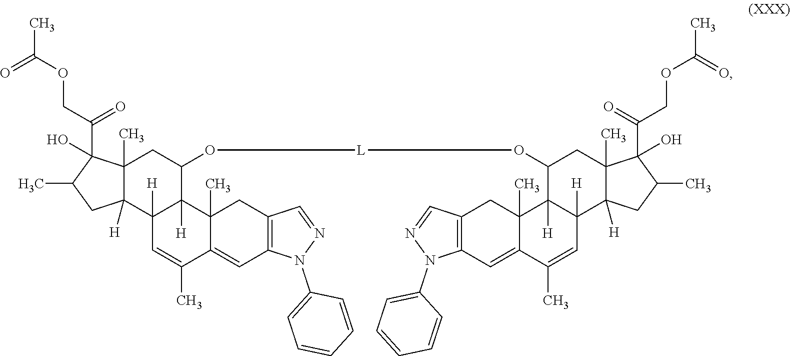

Wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXX) can be formed from the glucocorticoid steroid cortivazol.

In some embodiments, the steroid is a glucocorticoid steroid and the drug dimer is further described by the formula (XXXI):

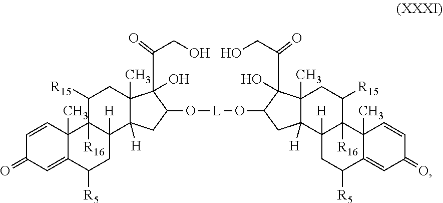

wherein R₅ represents H or a halogen atom; R₁₅ represents a halogen atom or OH; R₁₆ represents H or a halogen atom; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXI) can be formed from a glucocorticoid steroid selected from the group consisting of fluclorolone, fluocinolone, and triamcinolone.

In some embodiments, the steroid is a glucocorticoid steroid and the drug dimer is further described by the formula (XXXII):

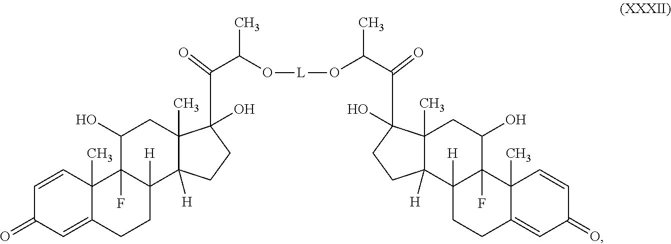

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXII) can be formed from fluperolone.

In some embodiments, the steroid is a glucocorticoid steroid and the drug dimer is further described by the formula (XXXIII):

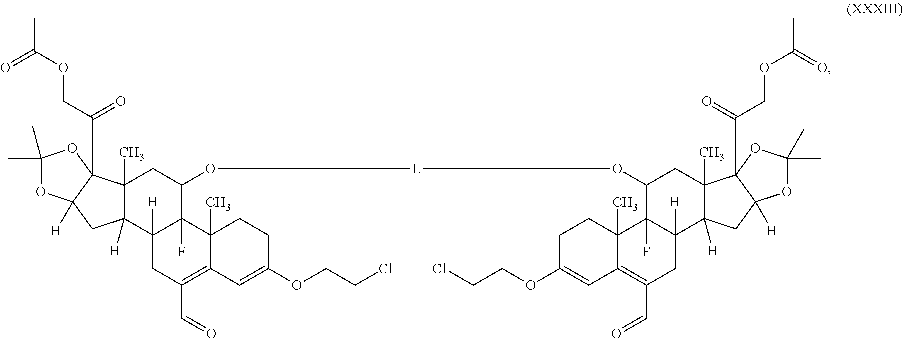

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXIII) can be formed from formocortal.

In particular embodiments, the steroid is a corticosteroid and the drug dimer is further described by the formula (XXXIV):

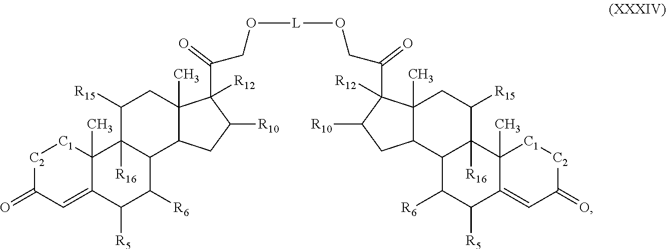

wherein the bond between C₁ and C₂ is a double or a single bond; R₁₆ represents H or a halogen atom; R₅ represents H, CH₃, or a halogen atom; R₁₂ represents H or a halogen atom; R₁₅ represents ═O or OH; R₁₂ and R₁₀ each, independently, represent —H, C₁₋₁₀ alkyl, —OH, —O-acyl, or R₁₂ and R₁₀ combine to form a cyclic acetal of formula (XVIII-a) wherein:

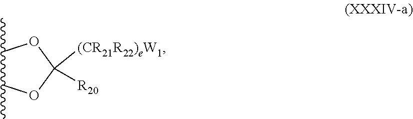

e is an integer from 0 to 6; R₂₀, R₂₁, and R₂₂ each, independently, represent H or C₁₋₁₀ alkyl; W₁ represents H or CH₃; L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXIV) can be formed from a corticosteroid selected from the group consisting of alclometasone, beclomethasone, betamethasone, betamethasone valerate, budesonide, chloroprednisone, cloprednol, corticosterone, cortisone, desonide, desoximerasone, dexamethasone, diflorasone, diflucortolone, enoxolone, flucloronide, flumethasone, flunisolide, fluocinolone acetonide, fluocortolone, fluprednisolone, flurandrenolide, halometasone, hydrocortisone, hydrocortisone butyrate, meprednisone, methylprednicolone, paramethasone, prednisolone, prednisone, prednival, prednylidene, triamcinolone, and triamcinolone acetonide.

In any of the above formulas (II)-(XXXIV), O—(R^{A})—O can be a radical of a polyol formed from a cyclitol, and sugar alcohol, or glycerin; or O—(R^{A})—O can be a radical formed from an alkane diol (e.g., a C₁₋₁₀ alkane diol), diethylene glycol, triethylene glycol, tetraethylene glycol, or pentaethylene glycol.

In particular embodiments, the steroid is a corticosteroid and the drug dimer is further described by the formula (XXXV):

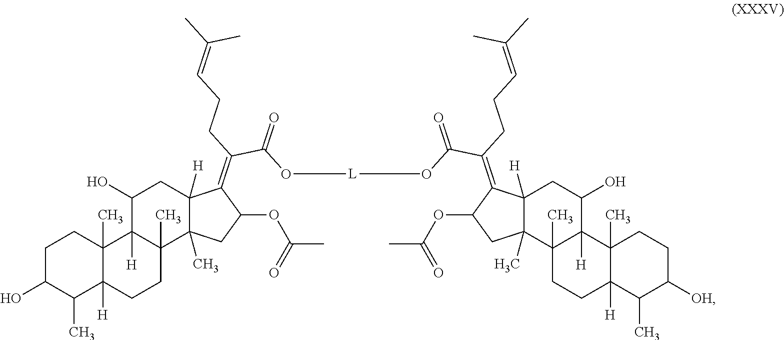

wherein L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (XXXV) can be formed from fusidic acid.

In some embodiments, the steroid is a corticosteroid and the drug dimer is further described by the formula (XXXVI):

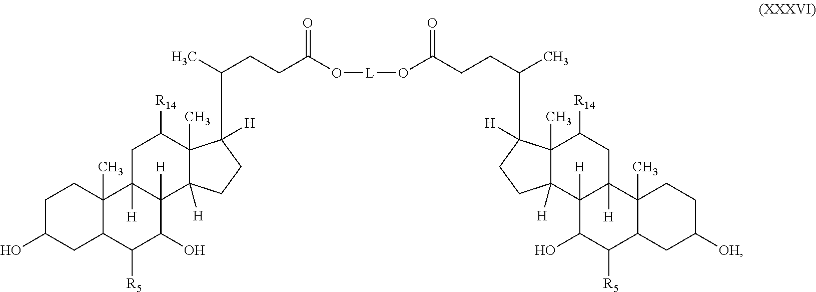

wherein R₅ represents H or C₁₋₆ alkyl; R₁₄ represents H or OH; and L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (XXXVI) can be formed from chenodeoxycholic acid, ursodeoxycholic acid, or obeticholic acid.

In some embodiments, the steroid is an anti-angiogenic steroid or an intraocular pressure (IOP) lowering steroid, and the drug dimer is further described by the formula (XXXVII):

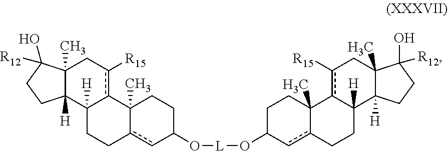

wherein R₁₂ represents —C(═O)CH₂OC(═O)CH₃, —C(═O)CH₂OH, or —C(═O)CH₃; R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXVII) can be formed from anecortave acetate, anecortave, 11-epicortisol, 17α-hydroxyprogesterone, tetrahydrocortexolone, or tetrahydrocortisol.

In some embodiments, the steroid is an anti-angiogenic steroid or an intraocular pressure (IOP) lowering steroid, and the drug dimer is further described by the formula (XXXVIII):

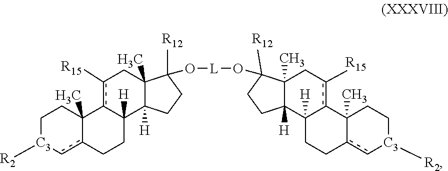

wherein the bond between C₃ and R₂ is a single or a double bond; R₂ represents OH or ═O; R₁₂ represents —C(═O)CH₂OC(═O)CH₃, —C(═O)CH₂OH, or —C(═O)CH₃, R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXVIII) can be formed from anecortave acetate, anecortave, 11-epicortisol, 17α-hydroxyprogesterone, tetrahydrocortexolone, or tetrahydrocortisol.

In some embodiments, the steroid is an anti-angiogenic steroid or an intraocular pressure (IOP) lowering steroid, and the drug dimer is further described by the formula (XXXIX):

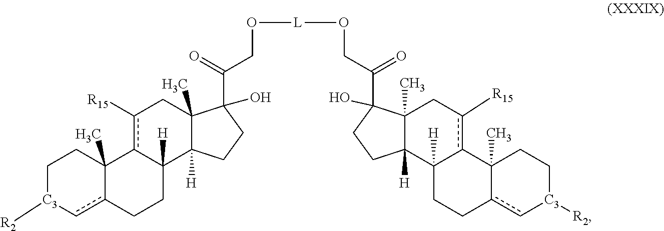

wherein the bond between C₃ and R₂ is a single or a double bond; R₂ represents OH or ═O; R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XXXIX) can be formed from anecortave, 11-epicortisol, tetrahydrocortexolone, or tetrahydrocortisol.

In some embodiments, the steroid is an anti-angiogenic steroid or an intraocular pressure (IOP) lowering steroid, and the drug dimer is further described by the formula (XL):

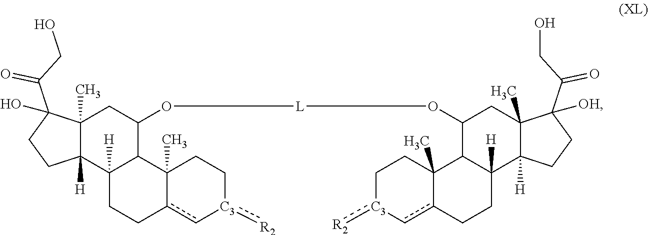

wherein the bond between C₃ and R₂ is a single or a double bond; R₂ represents OH or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XL) can be formed from 11-epicortisol or tetrahydrocortisol.

In some embodiments, the steroid is a benign steroid and the drug dimer is further described by the formula (XLI):

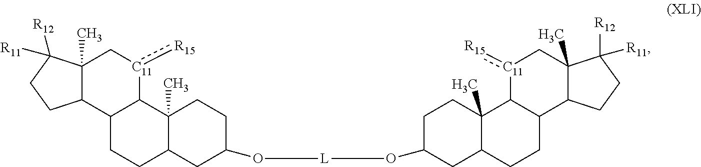

wherein the bond between C₁₁ and R₁₅ is a single or a double bond; R₁₁ represents H, OH, —C(═O)CH₂OH, or —C(═O)CH₃; R₁₂ represents H, OH, —C(═O)CH₂OH, or —C(═O)CH₃; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLI) can be formed from tetrahydrocortisone, tetrahydrodeoxycortisol, tetrahydrocorticosterone, 5α-dihydrocorticosterone, or 5α-dihydropregesterone.

In some embodiments, the steroid is a benign steroid and the drug dimer is further described by the formula (XLII):

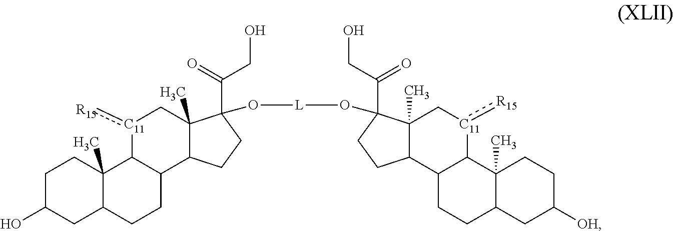

wherein the bond between C₁₁ and R₁₅ is a single or a double bond; R₁₅ represents H or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLII) can be formed from tetrahydrocortisone, or tetrahydrodeoxycortisol.

In some embodiments, the steroid is a benign steroid and the drug dimer is further described by the formula (XLIII):

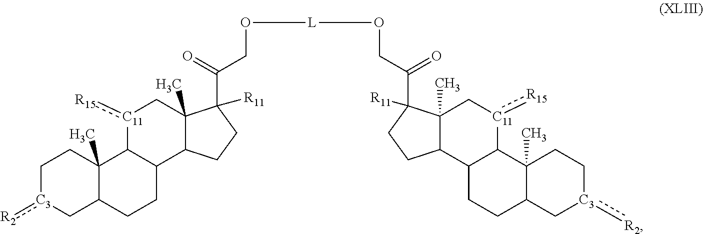

wherein the bond between C₃ and R₂, and C₁₁ and R₁₅ is a single or a double bond; R₂ represents OH or ═O; R₁₁ represents H, or OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLIII) can be formed from tetrahydrocortisone, tetrahydrodeoxycortisol, tetrahydrocorticosterone, or 5α-dihydrocorticosterone.

In some embodiments, the steroid is a benign steroid and the drug dimer is further described by the formula (XLIV):

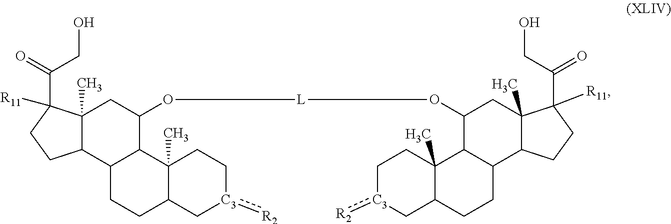

wherein the bond between C₃ and R₂ is a single or a double bond; R₂ represents OH or ═O; R₁₁ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLIV) can be formed from tetrahydrocortisone, tetrahydrocorticosterone, or 5α-dihydrocorticosterone.

In some embodiments, the steroid is a cholic acid-related bile acid steroid and the drug dimer is further described by the formula (XLV):

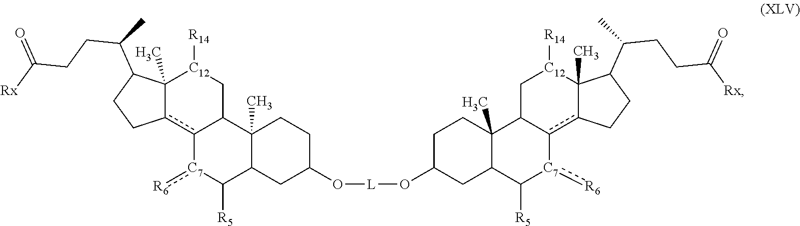

wherein the bond between C₇ and R₆, and C₁₂ and R₁₄ is a single or a double bond; Rx represents OH, —NHCH₂C(═O)OH, or —NHCH₂CH₂SO₂OH; R₂ represents OH or ═O; R₅ represents H or OH; R₆ represents H, ═O, or OH; R₁₄ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLV) can be formed from deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, or tauroursodeoxycholic acid.

In some embodiments, the steroid is a cholic acid-related bile acid steroid and the drug dimer is further described by the formula (XLVI):

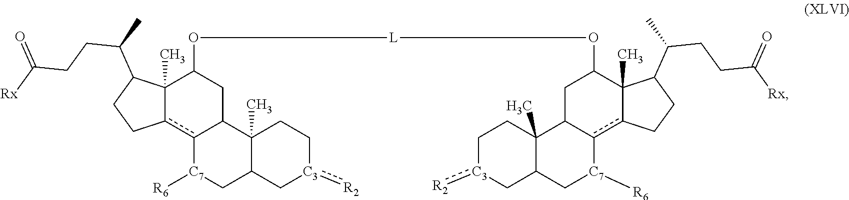

wherein the bond between C₃ and R₂, and C₇ and R₆ is a single or a double bond; Rx represents OH, —NHCH₂C(═O)OH, or —NHCH₂CH₂SO₂OH; R₂ represents OH or ═O; R₆ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLVI) can be formed from deoxycholic acid, apocholic acid, dehydrocholic acid, glycocholic acid, glycodeoxycholic acid, taurocholic acid, or taurodeoxycholic acid.

In some embodiments, the steroid is a cholic acid-related bile acid steroid and the drug dimer is further described by the formula (XLVII):

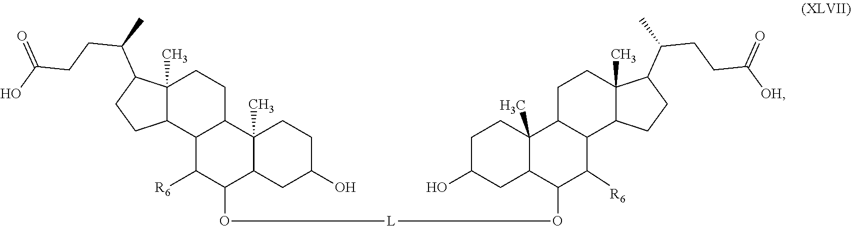

wherein R₆ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLVII) can be formed from hyodeoxycholic acid, α-muricholic acid, 3-muricholic acid, γ-muricholic acid, or ω-muricholic acid.

In some embodiments, the steroid is a cholic acid-related bile acid steroid and the drug dimer is further described by the formula (XLVIII):

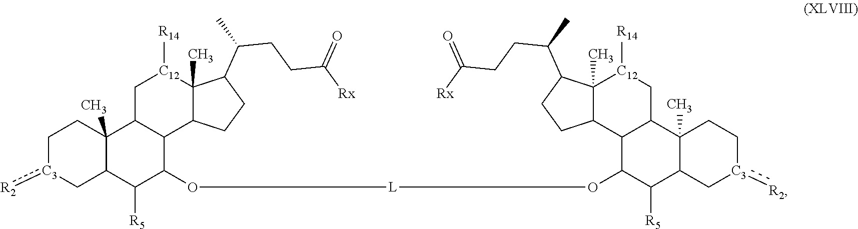

wherein the bond between C₃ and R₂, and C₁₂ and R₁₄ is a single or a double bond; Rx represents OH, —NHCH₂C(═O)OH, or —NHCH₂CH₂SO₂OH; R₂ represents OH or ═O; R₅ represents H or OH; R₁₄ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (XLVIII) can be formed from dehydrocholic acid, glycochenodeoxycholic acid, glycocholic acid, α-muricholic acid, β-muricholic acid, γ-muricholic acid, ω-muricholic acid, taurochenodeoxycholic acid, taurocholic acid, or tauroursodeoxycholic acid.

In some embodiments, the steroid is a cholic acid-related bile acid steroid and the drug dimer is further described by the formula (XLIX):

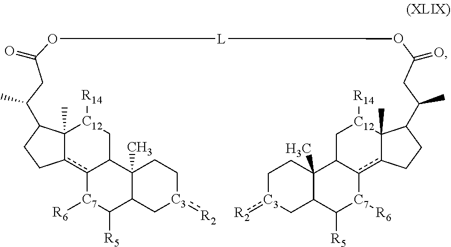

wherein the bond between C₃ and R₂, C₇ and R₆, and C₁₂ and R₁₄ is a single or a double bond; R₂ represents OH or ═O; R₅ represents H or OH; R₆ represents H, ═O, or OH; R₁₄ represents H, ═O, or OH; and L is —C(O)—(R^{A})—C(O)—, —(R^{A})—, or —C(O)—O—(R^{A})—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (XLIX) can be formed from deoxycholic acid, apocholic acid, dehydrocholic acid, hyodeoxycholic acid, lithocholic acid, α-muricholic acid, β-muricholic acid, γ-muricholic acid, or ω-muricholic acid.

In some embodiments, the steroid is a cholic acid-related bile acid steroid and the drug dimer is further described by the formula (L):

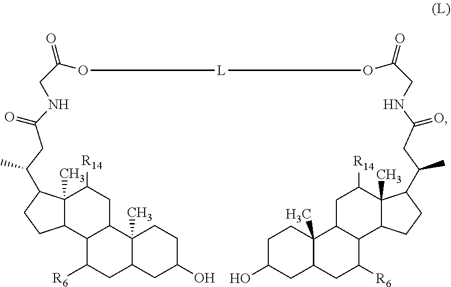

wherein R₆ represents H or OH; R₁₄ represents H or OH; and L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (L) can be formed from glycochenodeoxycholic acid, glycocholic acid, or glycodeoxycholic acid.

In some embodiments, the steroid is a steroid metabolite and the drug dimer is further described by the formula (LI):

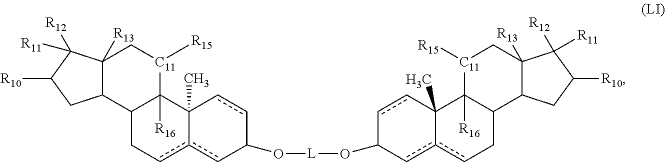

wherein the bond between C₁₁ and R₁₅ is a single or a double bond; R₂ represents OH or ═O; R₁₀ represents H or OH; R₁₁ represents H, OH, —C(═O)CH₂OH, —C(═O)OH, —C(═O)CH₂OH, or —C(═O)CH₃; R₁₂ represents H, OH, —C(═O)CH₂OH, —C(═O)OH, —C(═O)CH₂OH, or —C(═O)CH₃; R₁₃ represents —CH₂OH or —CH₃; R₁₅ represents H, OH, or ═O; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LI) can be formed from tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, or progesterone.

In some embodiments, the steroid is a steroid metabolite and the drug dimer is further described by the formula (LII):

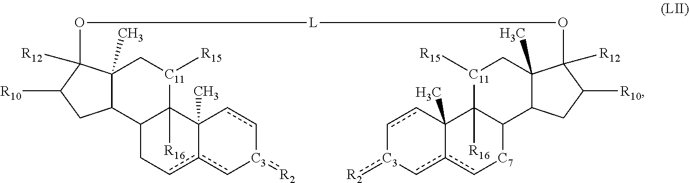

wherein the bond between C₃ and R₂, and C₁₁ and R₁₅ is a single or a double bond; R₂ represents OH or ═O; R₁₀ represents H or OH; R₁₂ represents —C(═O)CH₂OH, —C(═O)OH, —C(═O)CH₂OH, or —C(═O)CH₃; R₁₅ represents H, OH, or ═O; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LII) can be formed from tetrahydrotriamcinolone, cortienic acid, 17-hydroxypregnenolone, 17,21-dihydroxypregnenolone, or deoxycortisone.

In some embodiments, the steroid is a steroid metabolite and the drug dimer is further described by the formula (LIII):

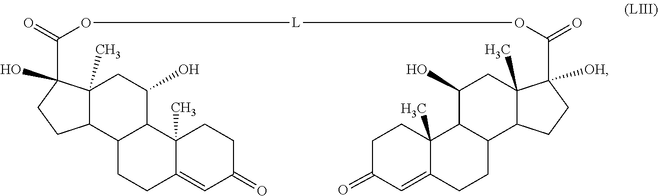

wherein L is —C(O)—(R^{A})—C(O)—, —(R^{A})—, or —C(O)—O—(R^{A})—O—C(O)— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms; or L is —O—(R^{A})—O— and R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from —O(CH₂CH₂O)ₙCH₂CH₂O—, —O(CH₂CH₂CH₂CH₂O)ₘCH₂CH₂CH₂CH₂O—, and —O(CH₂CH(CH₃)O)ₚCH₂CH(CH₃)O—; and n, m, and p are integers from 1 to 10. The drug dimer of formula (LIII) can be formed from cortienic acid.

In some embodiments, the steroid is a steroid metabolite and the drug dimer is further described by the formula (LIV):

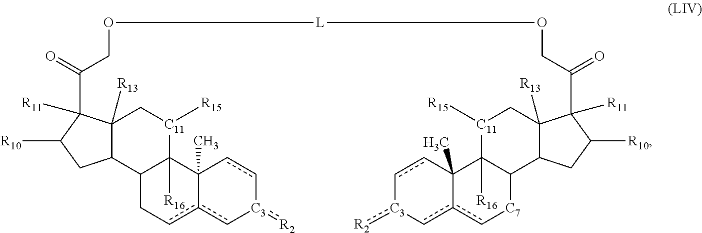

wherein the bond between C₃ and R₂, and C₁₁ and R₁₅ is a single or a double bond; R₂ represents OH or ═O; R₁₀ represents H or OH; R₁₁ represents H or OH; R₁₃ represents H, —CH₂OH, or —CH₃; R₁₅ represents H, OH, or ═O; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂-20 alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LIV) can be formed from tetrahydrotriamcinolone, 11-dehydrocorticosterone, 17,21-dihydroxypregnenolone, 18-hydroxycorticosterone, or 21-hydroxypregnenolone.

In some embodiments, the steroid is a steroid metabolite and the drug dimer is further described by the formula (LV):

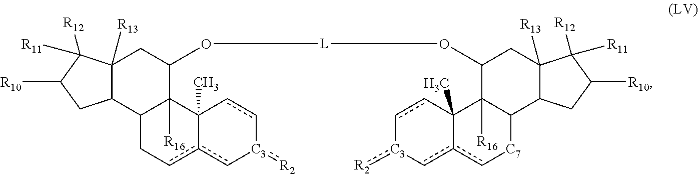

wherein the bond between C₃ and R₂ is a single or a double bond; R₂ represents OH or ═O; R₁₀ represents H or OH; R₁₁ represents H or OH; R₁₂ represents —C(═O)CH₂OH, —C(═O)OH, —C(═O)CH₂OH, or —C(═O)CH₃; R₁₃ represents H, —CH₂OH, or —CH₃; R₁₆ represents H or F; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LV) can be formed from tetrahydrotriamcinolone, cortienic acid, 11-dehydrocorticosterone, 11β-hydroxypregnenolone, ketoprogesterone, 18-hydroxycorticosterone, or deoxycortisone.

In some embodiments, the steroid is a steroid metabolite and the drug dimer is further described by the formula (LVI):

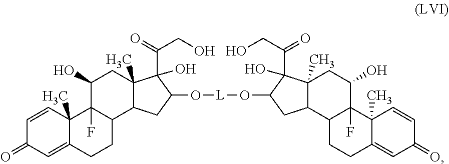

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LVI) can be formed from tetrahydrotriamcinolone.

In some embodiments, the steroid is a steroid metabolite and the drug dimer is further described by the formula (LVII):

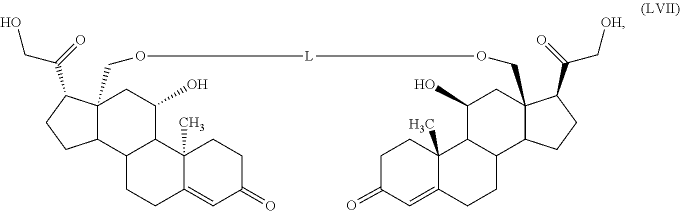

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LVII) can be formed from 18-hydroxycorticosterone.

In some embodiments, the steroid is a cholesterol-derivative and the drug dimer is further described by the formula (LVIII):

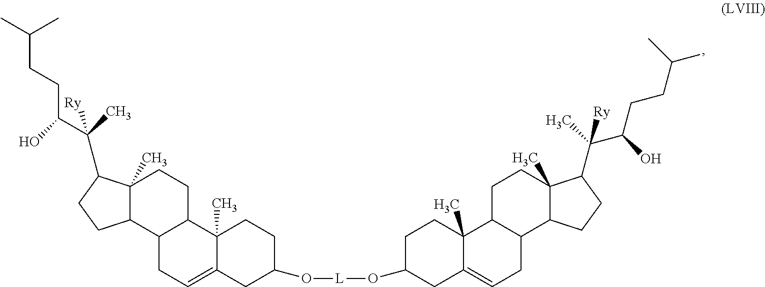

wherein Ry represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LVIII) can be formed from 22R-hydroxycholesterol or 20α-22R-dihydroxycholesterol.

In some embodiments, the steroid is a cholesterol-derivative and the drug dimer is further described by the formula (LIX):

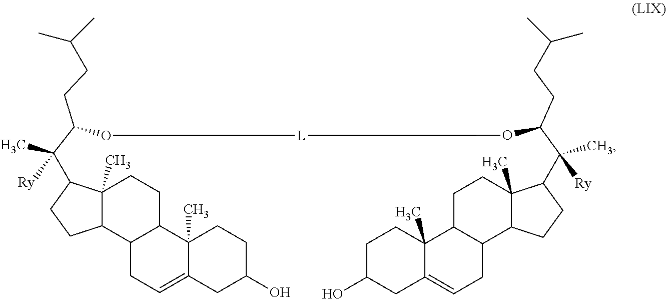

wherein Ry represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LIX) can be formed from 22R-hydroxycholesterol or 20α-22R-dihydroxycholesterol.

In some embodiments, the steroid is a cholesterol-derivative and the drug dimer is further described by the formula (LX):

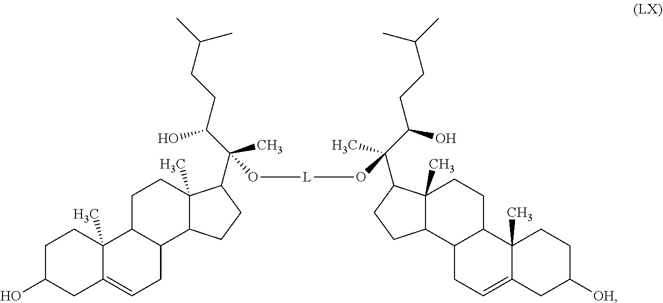

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LX) can be formed from 20α-22R-dihydroxycholesterol.

In some embodiments, the steroid is a neurosteroid and the drug dimer is further described by the formula (LXI):

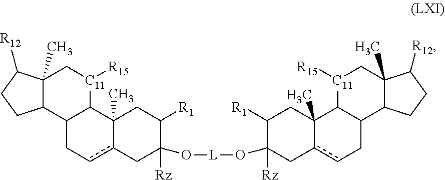

wherein the bond between C₁₁ and R₁₅ is a single or a double bond; Rz represents H or —CH₃; R₁ represents H or —OCH₂CH₃; R₂ represents OH or ═O; R₁₂ represents —OH, —C(═O)CH₃, —C(═O)CH₂OH, or —CH(CH₃)(CH₂)₂CH(OH)CH(CH₃)₂; R₁₅ represents H, —N(CH₃)₂, or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXI) can be formed from alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, or 24(S)-hydroxycholesterol.

In some embodiments, the steroid is a neurosteroid and the drug dimer is further described by the formula (LXII):

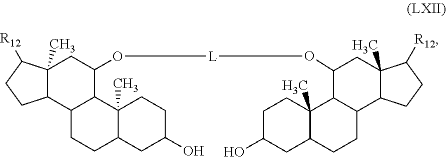

wherein R₁₂ represents —C(═O)CH₃, or —C(═O)CH₂OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXII) can be formed from alphaxalone or alphadolone.

In some embodiments, the steroid is a neurosteroid and the drug dimer is further described by the formula (LXIII):

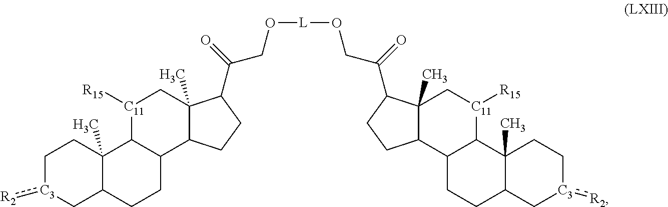

wherein the bond between C₃ and R₂, and C₁₁ and R₁₅ is a single or a double bond; R₂ represents OH or ═O; R₁₅ represents H or ═O; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXIII) can be formed from alphadolone, hydroxydione, or tetrahydrodeoxycorticosterone.

In some embodiments, the steroid is a neurosteroid and the drug dimer is further described by the formula (LXIV):

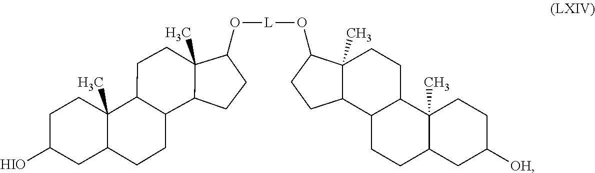

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXIV) can be formed from 3α-androstanediol.

In some embodiments, the steroid is a neurosteroid and the drug dimer is further described by the formula (LXV):

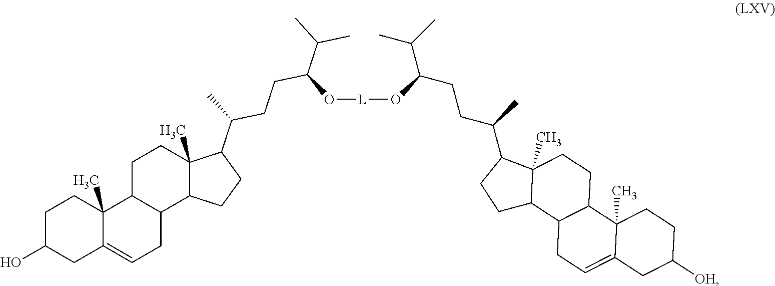

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXV) can be formed from 24(S)-hydroxycholesterol.

In some embodiments, the steroid is a pheromone and the drug dimer is further described by the formula (LXVI):

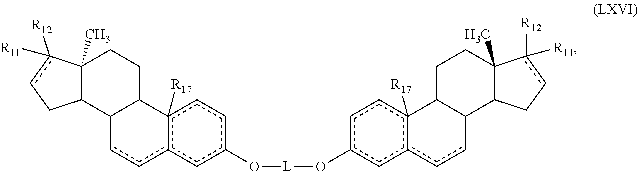

wherein R₂ represents OH or ═O; R₁₁ represents H, —C(═O)CH₃, —OC(═O)(CH₂)₄CH₃, or is absent; R₁₂ represents H, —C(═O)CH₃, —OC(═O)(CH₂)₄CH₃, or is absent; R₁₇ represents CH₃ or is absent; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXVI) can be formed from androstadienol, androstadienone, androstenol, androstenone, estratetraenol, 5-dehydroprogesterone, 6-dehydro-retroprogesterone, allopregnanolone, or hydroxyprogesterone caproate.

In some embodiments, the steroid is a progestin and the drug dimer is further described by the formula (LXVII):

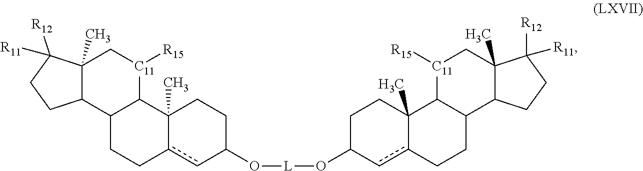

wherein the bond between C₁₁ and R₁₅ is a single or a double bond; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXVII) can be formed from allopregnone-3α,20α-diol, allopregnone-3β,20β-diol, allopregnane-3β,21-diol-11,20-dione, allopregnane-3β,17α-diol-20-one, 3,20-allopregnanedione, 3β,11β,17a, 203,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,20β-triol, allopregnane-3β,17α,21-triol-11,20-dione, allopregnane-3β,11β,21-triol-20-one, allopregnane-3β,17α,21-triol-20-one, allopregnane-3α-ol-20-one; allopregnane-3β-ol-20-one, pregnanediol, 3,20-pregnanedione, 4-pregnene-20,21-diol-3,11-dione, 4-pregnene-11β,17α,20β,21-tetrol-3-one, 4-pregnene-17α,20β,21-triol-3,11-dione, 4-pregnene-17α,20β,21-triol-3-one, or pregnenolone.

In some embodiments, the steroid is a progestin and the drug dimer is further described by the formula (LXVIII):

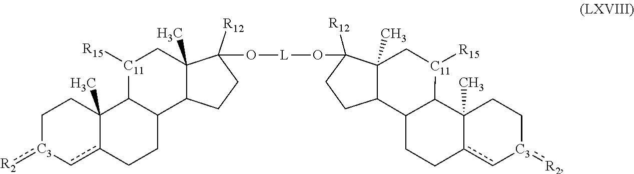

wherein the bond between C₃ and R₂, and C₁₁ and R₁₅ is a single or a double bond; R₂ represents OH or ═O; R₁₂ represents H, OH, —CH(OH)CH₃, —C(═O)CH₂OH, —C(═O)CH₃, or —CH(OH)CH₂OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXVIII) can be formed from allopregnane-3β,17α-diol-20-one, 3,20-allopregnanedione,3β,11β,17α,20β,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,20β-triol, allopregnane-3β,17α,21-triol-11,20-dione, allopregnane-3β,17α,21-triol-20-one, 4-pregnene-11β,17α,20β,21-tetrol-3-one, 4-pregnene-17α,20β,21-triol-3,11-dione, or 4-pregnene-17α,20β,21-triol-3-one.

In some embodiments, the steroid is a progestin and the drug dimer is further described by the formula (LXIX):

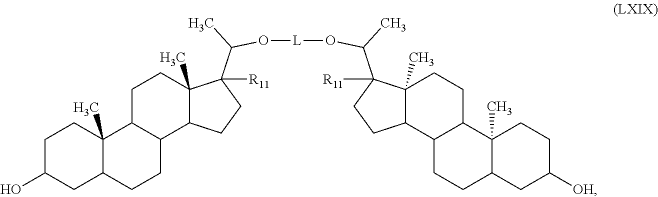

wherein R₁₁ represents H, OH, —CH(OH)CH₃, —C(═O)CH₂OH, —C(═O)CH₃, or —CH(OH)CH₂OH; R₁₅ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXIX) can be formed from allopregnone-3α,20α-diol, allopregnone-3β,20β-diol or allopregnane-3β,17α,20β-triol.

In some embodiments, the steroid is a progestin and the drug dimer is further described by the formula (LXX):

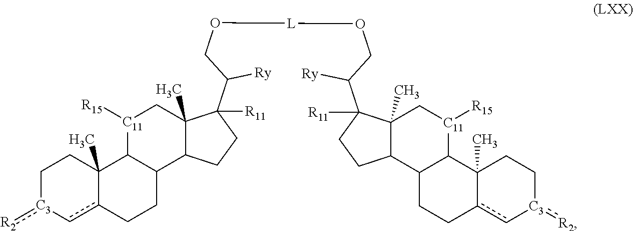

wherein the bond between C₃ and R₂, and C₁₁ and R₁₅ is a single or a double bond; Ry represents OH or ═O; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXX) can be formed from allopregnane-3β,21-diol-11,20-dione, 3,20-allopregnanedione,3β,11β,17α,20β,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,21-triol-11,20-dione, allopregnane-3β,11β,21-triol-20-one, allopregnane-3β,17α,21-triol-20-one, 4-pregnene-20,21-diol-3,11-dione, 4-pregnene-11β,17α,20β,21-tetrol-3-one, 4-pregnene-17α,20β,21-triol-3,11-dione, or 4-pregnene-17α,20β,21-triol-3-one.

In certain embodiments, the steroid is a progestin and the drug dimer is further described by the formula (LXXI):

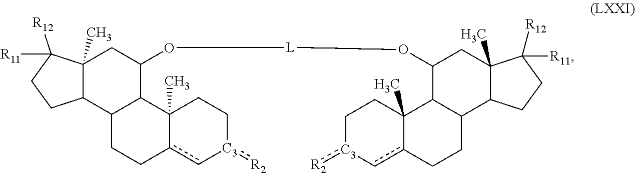

wherein the bond between C₃ and R₂ is a single or a double bond; R₂ represents OH or ═O; R₁₁ represents H, OH, —CH(OH)CH₃, —C(═O)CH₂OH, —C(═O)CH₃, or —CH(OH)CH₂OH; R₁₂ represents H, OH, —CH(OH)CH₃, —C(═O)CH₂OH, —C(═O)CH₃, or —CH(OH)CH₂OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂-20 alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or 0-(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXI) can be formed from allopregnane-3β,21-diol-11,20-dione, 3,20-allopregnanedione,3β,11β,17α,20β,21-pentol, allopregnane-3α,11β,17α,21-tetrol-20-one, allopregnane-3β,11β,17α,21-tetrol-20-one, allopregnane-3β,17α,21-triol-11,20-dione, allopregnane-3β,11β,21-triol-20-one, 4-pregnene-20,21-diol-3,11-dione, 4-pregnene-11β,17α,20β,2 some particular embodiments, the steroid is a progestin and the drug dimer is further described by the formula (LXXII):

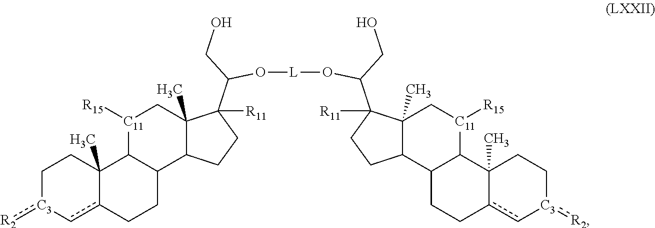

wherein the bond between C₃ and R₂, and C₁₁ and R₁₅ is a single or a double bond; R₂ represents OH or ═O; R₁₁ represents H or OH; R₁₅ represents H, ═O, or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXII) can be formed from 3,20-allopregnanedione,3β,11β,17α,20β,21-pentol, allopregnane-3β,17α,20β,21-tetrol, 4-pregnene-20,21-diol-3,11-dione, 4-pregnene-11β,17α,20β,21-tetrol-3-one, 4-pregnene-17α,20β,21-triol-3,11-dione, or 4-pregnene-17α,20β,21-triol-3-one.

In some embodiments, the steroid is other steroid and the drug dimer is further described by the formula (LXXIII):

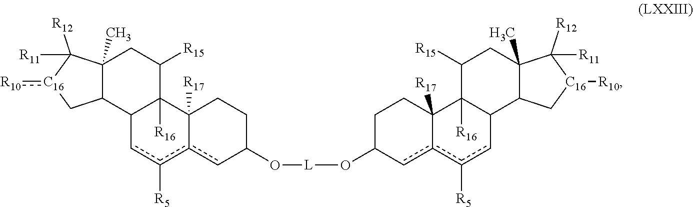

wherein the bond between C₁6 and R₁₀ is a single or a double bond; R₂ represents OH or ═O; R₅ represents H, Cl, or —CH₃; R₁₀ represents H or ═CH₂; R₁₁ represents H, OH, —CH₃, —C(═O)CH₃, —C(═O)CH₂OC(═O)CH₃, or —OC(═O)CH₃; R₁₂ represents H, OH, —CH₃, —C(═O)CH₃, —C(═O)CH₂OC(═O)CH₃, or —OC(═O)CH₃; R₁₅ represents H or OH; R₁₆ represents F or H; R₁ represents H or —CH₃; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXIII) can be formed from flugestone, prebediolone, chlormadinone acetate, medrogestone, or segesterone acetate.

In some embodiments, the steroid is other steroid and the drug dimer is further described by the formula (LXXIV):

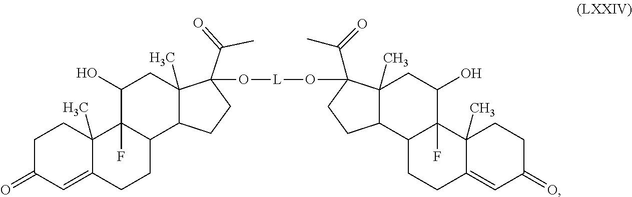

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXIV) can be formed from flugestone.

In some embodiments, the steroid is another steroid and the drug dimer is further described by the formula (LXXV):

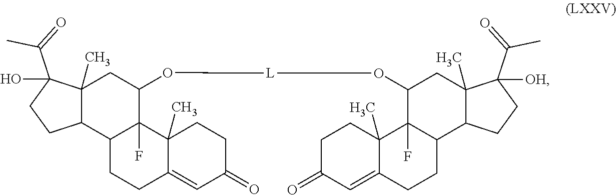

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXV) can be formed from flugestone.

In some embodiments, the steroid is a mineralocorticoid steroid, and the drug dimer is further described by the formula (LXXVI):

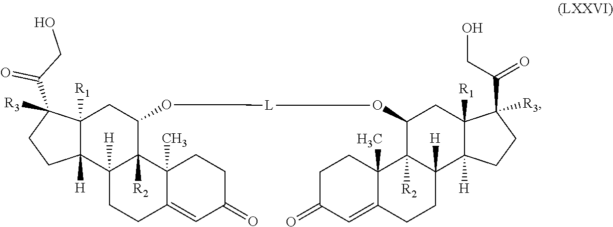

wherein R₁ is C(O)H or CH₃; R₂ represents H or F; R₃ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXVI) can be formed from fludrocortisone or aldocortisone.

In some embodiments, the steroid is a mineralocorticoid steroid, and the drug dimer is further described by the formula (LXXVII):

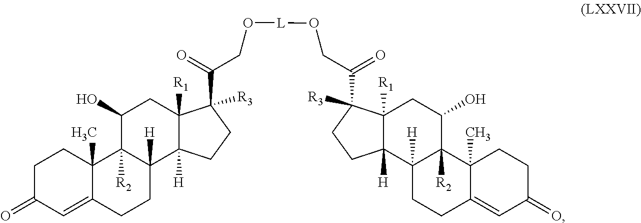

wherein R₁ is C(O)H or CH₃; R₂ represents H or F; R₃ represents H or OH; and L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXVII) can be formed from fludrocortisone or aldocortisone.

In some embodiments, the steroid is a mineralocorticoid steroid, and the drug dimer is further described by the formula (LXXVIII):

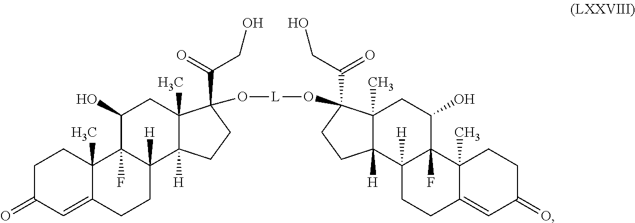

wherein L is —C(O)O—(R^{A})—OC(O)—, —C(O)—OC(O)—(R^{A})—C(O)O—C(O)—, or —C(O)—(R^{B})—C(O)O—(R^{A})—OC(O)—(R^{B})—C(O)—; R^{A} is selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms, or O—(R^{A})—O is a radical of a polyol and includes at least one free hydroxyl group or O—(R^{A})—O is selected from: —O(CH₂CH₂O)ₙCH₂CH₂O—, —O(CH₂CH₂CH₂CH₂O)ₘCH₂CH₂CH₂CH₂O—, or —O(CH₂CH(CH₃)O)ₚCH₂CH(CH₃)O—; n, m, and p are integers from 1 to 10; and each R^{B} is independently selected from C₁₋₂₀ alkylene, a linear or branched heteroalkylene of 1 to 20 atoms, a linear or branched C₂₋₂₀ alkenylene, a linear or branched C₂₋₂₀ alkynylene, a C₅₋₁₀ arylene, a cyclic system of 3 to 10 atoms. The drug dimer of formula (LXXVIII) can be formed from fludrocortisone.

### Processing Methods

In some embodiments, articles or drug depots provided herein are formed using methods provided herein such as, for example, heat processing or solvent processing of the drug dimer of formula (I). Heat processing can include annealing after the surface coating is formed. Heat processing can also include heat molding, injection molding, extrusion, 3D printing, melt electrospinning, fiber spinning, fiber extrusion, blow molding, and/or annealing after the drug depot is formed. Solvent processing may include coating, micro printing, emulsion processing, dot printing, micropatterning, dip coating, spray coating, stamp coating, brush coating, drop and drag coating, electrospraying, and electrospinning to produce one or more layers at a surface. Solvent processing may also include electrospraying and electrospinning the drug depot onto one or more layers on the surface or around the medical device or a component of the medical device. Solvent-free coating methods may also be used including powder coating followed by annealing. Solvent-free methods may also be used including annealing a powder to form a drug depot of the invention. In some embodiments, a mold is used to produce a drug depot of the desired shape. For example, the compounds of the disclosure can be heat processed to form a melt, and poured into a mold to produce, upon cooling, a shaped drug depot.

In some embodiments, a portion or side of the article is masked to produce a single-sided or patterned coating (e.g., a checkerboard pattern, dot pattern, or striped pattern) on an article.

### Electrospraying Method

In some embodiments, the compounds provided herein are dissolved in a solvent (e.g., acetone) at concentrations ranging from, e.g., 10-30% w/v, and are electrosprayed onto a surface to form surface coatings or drug depots of the invention. The solutions can be loaded into a syringe and can be injected at a particular rate, e.g., 0.5 mL/h, onto a stationary collection plate. Between the needle and collecting surface, a potential difference of, e.g., 18 kV, can be maintained. Exemplary concentrations of 10 to 30% w/v, depending upon the desired thickness of the drug depot or material being layered onto the surface.

### Fiber Spinning Methods

In some embodiments, the drug depot of the disclosure is a fibrous pouch around the medical device or a component of the device or a fibrous wafer inserted adjacent to the medical device. The fibrous meshes can have aligned and unaligned morphologies and are prepared by electrospinning. The pharmaceutical compositions of the disclosure are dissolved in a solvent (e.g., THF, or 1:1 ratio of DCM/THF). The solutions may be injected from a syringe at a particular rate, e.g., 0.5 mL/h, onto the device or a component of the medical device rotating at a particular rotational speed, e.g., 1150 rpm, to obtain aligned fibers around the device or component, or 30 rpm, to obtain unaligned fibers around the device or component, or onto a stationary device or component to obtain unaligned fibers on only one side of the device or component. A potential difference (e.g., 18 kV or 17 kV) can be maintained between the needle and collecting surface for aligned and random fibers.

In other embodiments, fibers are prepared either from the melt at elevated temperatures, the glassy state intermediate, or from solution by dissolving the pharmaceutical compositions of the disclosure in a solvent (e.g., DCM, THF, or chloroform). As used herein, melt spinning describes heat processing from the melt state, heat spinning describes heat processing from the glassy state, and wet, dry, and gel spinning describe solution processing.

The viscous melt, intermediate, or solution can be fed through a spinneret and fibers may be formed upon cooling (melt or heat spinning) or following solvent evaporation with warm air as the compound exits the spinneret (dry spinning). Wet spinning and gel spinning, performed according to methods known in the art, may also be used to produce the fibers of the disclosure. Heat spinning describes a process that is essentially the same as the melt spinning process, but performed with the glassy state intermediate and heated above the glass transition temperature (Tg) to get the viscous fluid to extrude/spin instead of the melt.

### Extrusion Method

In some embodiments, cylinders made from the pharmaceutical composition may injected adjacent to a medical device upon implantation and/or at various time points after device implantation and may be formed by heat extrusion. The pharmaceutical composition may be loaded into a hot melt extruder, heated to a temperature above the melting point (for crystalline compositions) or glass transition temperature (for pre-melted or amorphous compositions), and extruded using a light compressive force to push the material through the nozzle and a light tensile force to pull the material out of the extruder.

The extrudate may be cut to the desired length for appropriate drug dosing for the indication of interest.

Low Temperature Processing Using Intermediate Glassy State Articles or Drug Depots

In certain embodiments, the dimer has a limited window (e.g., short timeframe of seconds to minutes) of thermal stability, whereby the purity of the dimer is minimally affected at elevated temperatures. In some embodiments, it is beneficial to make an intermediate glassy state form of the surface coating or drug depot. This can be accomplished by heat or solvent processing to remove or reduce the crystallinity of the material to form a glassy state composition. The glassy state composition is subsequently heat processed at a lower temperature (e.g., processing just above the glass transition temperature (Tg), and below the melt temperature (Tm)). This can provide a longer timeframe for heat processing the glassy state material into the final surface coating or drug depot (e.g., annealing), while reducing the impact of processing conditions on the purity of the prodrug dimer in the article or drug depot.

Exemplary processing details are provided in the Examples.

### Drug Delivery

In some embodiments, the compositions (e.g., pharmaceutical compositions), articles, and drug depots provided herein are tailored for optimal delivery of a drug (e.g., release the drug from an article or drug depot provided herein in a controlled manner, for example, by surface erosion). The surface erosion mechanism of drug release may allow the coating or drug depot to maintain its physical form (shape), while gradually decreasing in size as the surface erodes (e.g., like a bar of soap), rather than bulk erosion that is characteristic of some polymer-based drug release vehicles (e.g., polylactic/glycolic acid). This may inhibit burst release and reduce the formation of inflammatory particulates. The drug can be controlled to be delivered over a desired period of time. A slower and steadier rate of delivery (e.g., release of less than 10% of D1 or D2 (as a percentage of the total drug, D1 or D2, present in the surface coating or drug depot in prodrug form) at 37° C. in 100% bovine serum over 5 days) may in turn result in improved device function and longevity, a reduction in the frequency pharmaceuticals must be administered, and may reduce or avoid systemic side effects associated with use of the drug. Drug release can also be tailored to avoid side effects of slower and longer release of the drug by engineering the surface coating or drug depot to provide steady release over a comparatively shorter period of time. Depending on the indication, the medical device, and the drug, the drug release can be tailored for dose and duration appropriate to the indication of interest.

In some embodiments, the rate of release of a drug depends on the drug composition of the drug dimer. Drug release rate from the formed object of the drug dimer can be modulated by the cleavage of a drug-linker bond through hydrolysis or enzymatic degradation. In some embodiments, the linker can affect drug release rate. In some embodiments, the drug release rate is controlled by the selection of the functional group on the drug to conjugate through to the linker, for example, a primary vs. a secondary steroid hydroxyl group. The rate of release of a given drug from a drug dimer may also depend on the quantity of the loaded drug dimer as a percent of the final drug dimer formulation, e.g., by using a pharmaceutical excipient (e.g., bulking agent/excipient) or a second steroid drug (e.g., active or benign) as a homodimer mixture, or within the same molecule as a heterodimer that acts as a bulking agent. In some embodiments, drug release is tailored based on the solubility of drug dimer (e.g., through selection of appropriate drug and/or linker) that will influence the rate of surface erosion (e.g., dissolution/degradation) from the article or drug depot. In other embodiments, drug release is affected by changes in the thickness and/or surface area of the formulation, e.g., by applying layers until the desired coating thickness is obtained, or by changing the size of the drug depot or the surface area to which the coating is applied. By adjusting the vide supra factors, dissolution, degradation, diffusion, and controlled release may be varied over wide ranges. For example, release may be designed to be initiated over minutes to hours, and may extend over the course of days, weeks, months, or years.

### Uses and Pharmaceutical Compositions and Drug Depots

In some embodiments, the dimers provided herein are applied to an implantable drug delivery device (or, e.g., a drug depot) with minimal additives. This may achieve a local, sustained release and a local biological effect, while minimizing a systemic response. In some embodiments, when present, the additives are in small amounts and do not affect the physical or bulk properties. In some embodiments, when present, the additives do not alter the drug release properties from the pharmaceutical composition but rather act to improve processing of the prodrug dimer into the device coating or drug depot. In some embodiments, the pharmaceutical compositions contain additives such as a plasticizer (e.g., to reduce thermal transition temperatures), an antioxidant (e.g., to increase stability during heat processing), a binder (e.g., to add flexibility to the fibers), a bulking agent (e.g., to reduce total drug content), a lubricant, a radio-opaque agent, or mixtures thereof. The additives may be present at 30% (w/w), e.g., 20% (w/w), 10% (w/w), 7% (w/w), 5% (w/w), 3% (w/w), 1% (w/w), 0.5% (w/w), or 0.1% (w/w). Examples of plasticizers are polyols, e.g., glycerol, ethylene glycol, diethylene glycol, triethylene glycol, tetraethylene glycol, polyethylene glycol, propylene glycol, triacetin, sorbitol, mannitol, xylitol, fatty acids, monosaccharides (e.g., glucose, mannose, fructose, sucrose), ethanolamine, urea, triethanolamine, vegetable oils, lecithin, or waxes. Exemplary antioxidants are glutathione, ascorbic acid, cysteine, or tocopherol. The binders and bulking agents can be, e.g., polyvvinylpyrrolidone (PVP), starch paste, pregelatinized starch, hydroxypropyl methyl cellulose (HPMC), carboxymethyl cellulose (CMC), or polyethylene glycol (PEG) 6000.

### Sterilization of Formulations

Generally, it is desired that a medical device is sterile before or upon administration to a subject. In some embodiments, a sterile formulation is essentially free of pathogenic microorganisms, such as bacteria, microbes, fungi, viruses, spores, yeasts, molds, and others generally associated with infections. In some embodiments, medical devices containing the surface coatings or drug depots provided herein are subjected to an aseptic process and/or other sterilization process. In some embodiments, an aseptic process involves sterilizing the components of the device, the final device, and/or container closure of the combination product through a process such as heat, gamma irradiation, ethylene oxide, or filtration and then combining in a sterile environment. In some cases, an aseptic process is preferred. In other embodiments, terminal sterilization is preferred.

Treatment Methods and Therapy with Drug Depots in Combination with Medical Devices

In certain instances, the surface coatings or drug depots provided herein are used in combination with an implantable medical device (e.g., employed in the fields of ophthalmology, oncology, laryngology, endocrinology and metabolic diseases, rheumatology, urology, neurology, cardiology, dental medicine, dermatology, otology, post-surgical medicine, orthopedics, pain management, and gynecology).

In some embodiments, a compound provided herein is selected for the desired property, such as, for example, corticosteroid dimers for use in treating inflammatory diseases or conditions or for reducing inflammation or foreign body response due to device itself or device implantation procedure; or the use of antibiotic steroid dimers for treating an infection or preventing device-related infections.

### Surgical Procedures

In some embodiments, the surface coatings or drug depots provided herein are used in conjunction with a surgical procedure. For example, a device bearing a surface coating or drug depot provided herein can be implanted at a surgical site to reduce the risk of infection, inflammation, or to monitor a condition (such as when combined for use in an implantable sensor).

### Articles and Implantable Medical Devices

Any article can be coated with the surface coatings of the invention. For example, articles suitable for contact with bodily fluids, such as medical can be coated using the compositions described herein. For example, the article can be blood dwelling medical device (e.g., a heart valve, vascular stent, endovascular coil, or catheter), urine dwelling medical device (e.g., a drainage catheter or ureteral stent), and/or subcutaneously dwelling medical device (e.g., an implantable sensor). The duration of contact may be short, for example, as with surgical instruments or long-term use articles such as implants.

Any implantable medical device can be used in combination with the surface coating or drug depots of the invention. For example, the implantable medical device can be blood dwelling medical device (e.g., a heart valve, vascular stent, endovascular coil, or catheter), urine dwelling medical device (e.g., a drainage catheter or ureteral stent), and/or subcutaneously dwelling medical device (e.g., an implantable sensor). The implantable medical devices include, without limitation, catheters, guide wires, vascular stents, probes, sensors, transdermal patches, vascular patches, orthopedics (e.g., screws and plates), hernia mesh, ophthalmological devices (i.e., punctal plug, contact lenses, minimally invasive glaucoma surgery (MIGS) devices, intraocular lens), vaginal slings, and tubing. For example, the article can be selected from dental devices, dental implants, drug delivery devices, grafts, stents, implantable cardioverter-defibrillators, heart valves, vena cava filters, endovascular coils, catheters, shunts, wound drains, drainage catheters, infusion ports, cochlear implants, endotracheal tubes, tracheostomy tubes, ventilator breathing tubes, implantable sensors, ophthalmic devices, orthopedic devices, periodontal implants, breast implants, penile implants, maxillofacial implants, cosmetic implants, valves, appliances, scaffolding, suturing material, needles, hernia repair meshes, tension-free vaginal tape and vaginal slings, prosthetic neurological devices, ear tubes, a wound dressing, a bandage, a gauze, a tape, a pad, a sponge, a contraceptive device, and feminine hygiene products.

In some embodiments, the implantable device is selected from dental devices, dental implants, drug delivery devices, grafts, stents, implantable cardioverter-defibrillators, heart valves, vena cava filters, endovascular coils, catheters, shunts, wound drains, drainage catheters, infusion ports, cochlear implants, endotracheal tubes, tracheostomy tubes, ventilator breathing tubes, implantable sensors, ophthalmic devices, orthopedic devices, periodontal implants, breast implants, penile implants, maxillofacial implants, cosmetic implants, valves, appliances, scaffolding, suturing material, needles, hernia repair meshes, tension-free vaginal tape and vaginal slings, prosthetic neurological devices, ear tubes, a wound dressing, a bandage, a gauze, a tape, a pad, a sponge, a contraceptive device, feminine hygiene products, prostheses, pacemakers, electrical leads, defibrillators, artificial hearts, ventricular assist devices, anatomical reconstruction prostheses, artificial heart valves, heart valve stents, pericardial patches, surgical patches, coronary stents, vascular grafts, vascular and structural stents, vascular or cardiovascular shunts, biological conduits, pledges, sutures, annuloplasty rings, stents, staples, valved grafts, dermal grafts for wound healing, orthopedic spinal implants, ophthalmic implants, intrauterine devices, maxial facial reconstruction plating, intraocular lenses, MIGS devices, clips, and sternal wires.

In some embodiments, surface coatings provided herein are used as a surface covering for an article (e.g., where the polymers or admixtures are of a type capable of being formed into 1) a self-supporting structural body, 2) a film; or 3) a fiber, preferably woven or knit). The composition may comprise a surface or in whole or in part of the article (e.g., a biomedical device or device of general biotechnological use). In some embodiments, applications include cardiac assist devices, tissue engineering polymeric scaffolds and related devices, cardiac replacement devices, cardiac septal patches, intra aortic balloons, percutaneous cardiac assist devices, extra-corporeal circuits, A-V fistual, dialysis components (tubing, filters, membranes, etc.), aphoresis units, membrane oxygenator, cardiac by-pass components (tubing, filters, etc.), pericardial sacs, contact lens, cochlear ear implants, sutures, sewing rings, cannulas, contraceptives, syringes, o-rings, bladders, penile implants, drug delivery systems, drainage tubes, pacemaker lead insulators, heart valves, blood bags, coatings for implantable wires, catheters, vascular stents, angioplasty balloons and devices, bandages, heart massage cups, tracheal tubes, mammary implant coatings, artificial ducts, craniofacial and maxillofacial reconstruction applications, ligaments, fallopian tubes.

In some embodiments, drug depots provided herein are retained by or affixed to the implantable medical device. For example, the drug depot can be glued to the surface of the implantable medical device or retained by a screw, washer, or bolt. In some embodiments, the implantable medical device includes a reservoir for holding a drug depot provided herein or can be held on its own (e.g. a fibrous pouch around the device or component). In another embodiment, the drug depot is implanted adjacent to (e.g., separately from) the implantable medical device (e.g., at the time of surgical implantation and/or subsequent to the implantation of the medical device).

The medical device can be an implanted device, percutaneous device, or cutaneous device. Implanted devices include articles that are fully implanted in a patient, e.g., are completely internal. Percutaneous devices include items that penetrate the skin, thereby extending from outside the body into the body. Cutaneous devices are used superficially. Implanted devices include, without limitation, prostheses such as pacemakers, electrical leads such as pacing leads, defibrillarors, artificial hearts, ventricular assist devices, anatomical reconstruction prostheses such as breast implants, artificial heart valves, heart valve stents, pericardial patches, surgical patches, coronary stents, vascular grafts, vascular and structural stents, vascular or cardiovascular shunts, biological conduits, pledges, sutures, annuloplasty rings, staples, valved grafts, dermal grafts for wound healing, orthopedic spinal implants, orthopedic pins, intrauterine devices, urinary stents, maxial facial reconstruction plating, dental implants, intraocular lenses, clips, sternal wires, bone, skin, ligaments, tendons, and combination thereof. Percutaneous devices include, without limitation, catheters or various types, cannulas, drainage tubes such as chest tubes, surgical instruments such as forceps, retractors, needles, and gloves, and catheter cuffs. Cutaneous devices include, without limitation, burn dressings, wound dressings and dental hardware, such as bridge supports and bracing components.

In some embodiments, an implantable medical device provided herein is generally structured from a base metallic, ceramic, or polymeric platform in a solid-state format. In some embodiment, the composition provided herein, either alone or as an admixture, controls the release of a therapeutic agent from the device for local drug delivery applications.

The following examples, as set forth below and as summarized in Table 3. are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Compound 1 (Dex-TEG-Dex) can be Synthesized, Coated onto Different Substrates, and Shows Sustained Release from the Coated Surfaces

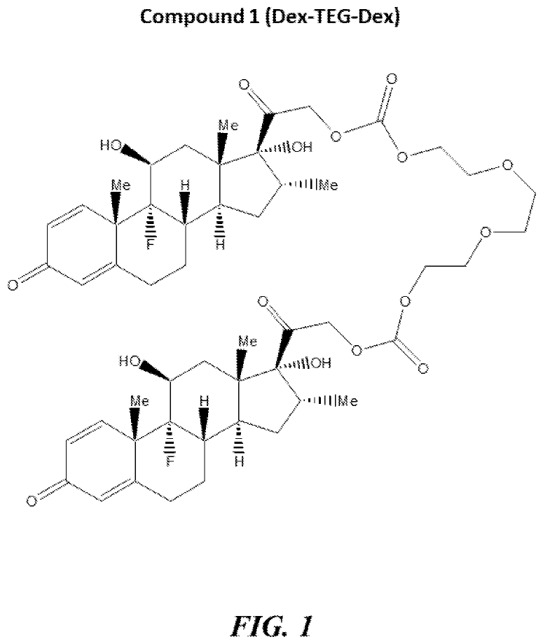

Dexamethasone (1 mol equivalent) was suspended in dichloromethane on an ice bath and triethylamine (2 mol equivalent) and triethylene glycol bis(chloroformate) (0.6 mol equivalent) were added to the mixture. The ice bath warmed to room temperature and the reaction was stirred overnight. The solvent was removed, and the solid residue was purified by column chromatography. Product was recrystallized from acetonitrile twice to give Compound 1 (FIG. 1) as an off-white crystalline solid.

Compound 1: HPLC (mobile phase: H₂O/TFA and MeCN/TFA) 31.7 min; Elemental analysis: Anal. Calcd for C₅₂H₆₈F₂O₁₆: C, 63.27; H, 6.94; N, 0.00; Cl, 0.00 Found: C, 62.62; H, 6.84; N, <0.50; Cl <100 ppm. ¹H NMR (400 MHz, DMSO-d₆) δ (ppm) 0.80 (d, J=7 Hz, 6H, 2×C16 α-CH₃); 0.90 (s, 6H, 2×C18-CH₃); 1.08 (m, 2H, 2×C16-H); 1.35 (m, 2H, 2×C14-H); 1.49 (s, 6H, 2×C19-CH₃); 1.54 (q, J=13 Hz, 2H, 2×C13-H); 1.64 (q, J=11 Hz, 2H, 2×C15-CH₂); 1.77 (m, 2H, 2×C15-CH₂); 2.15 (m, 4H, 2×C6-CH₂); 2.32 (m, 4H, 2×C7-CH₂); 2.62 (m, 2H, 2×C12-CH₂); 2.89 (m, 2H, 2×C12-CH₂); 3.57 (s, 4H, 2×TEG OCH₂); 3.65 (m, 4H, 2×TEG OCH₂); 4.15 (m, 2H, 2×OCH); 4.22 (m, 4H, 2×TEG OCH₂); 4.79 (d, 2H, AB, J=18.5 Hz, 2H, C21-CH₂O—); 5.09 (d, 2H, AB, J=18.5 Hz, 2H, C21-CH₂O—); 5.18 (s, 2H, C17-OH); 5.40 (d, 2H, J=4.5 Hz, C11-OH); 6.01 (d, 2H, J=1.9 Hz, 2×alkene C4-CH); 6.23 (dd, 2H, J=10.1 and 1.9 Hz, CH, 2×alkene C2-CH); 7.29 (d, 2H, C1-CH 2×alkene CH, 10.1 Hz, 2H). MS (ESI+) m/z: [M+H]+ Calcd for C₅₂H₆₉F₂O₁₆ 987.46; Found 987.46.

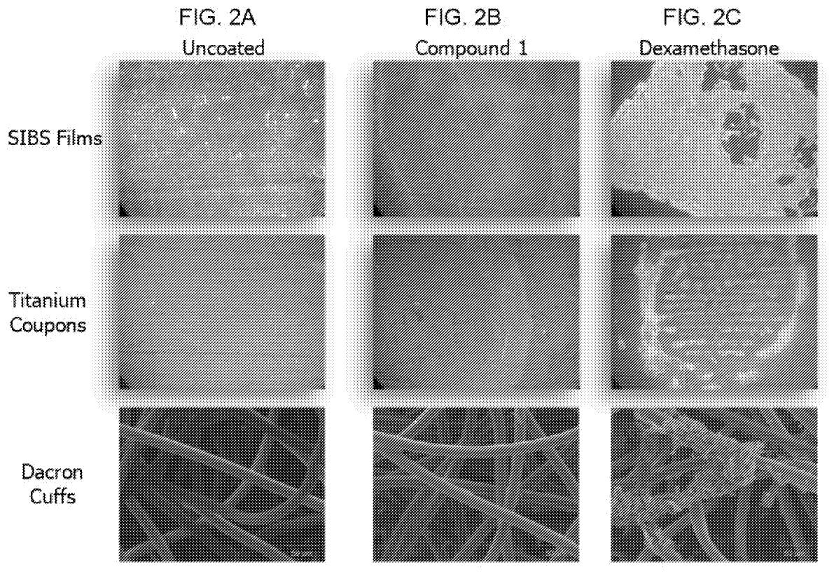

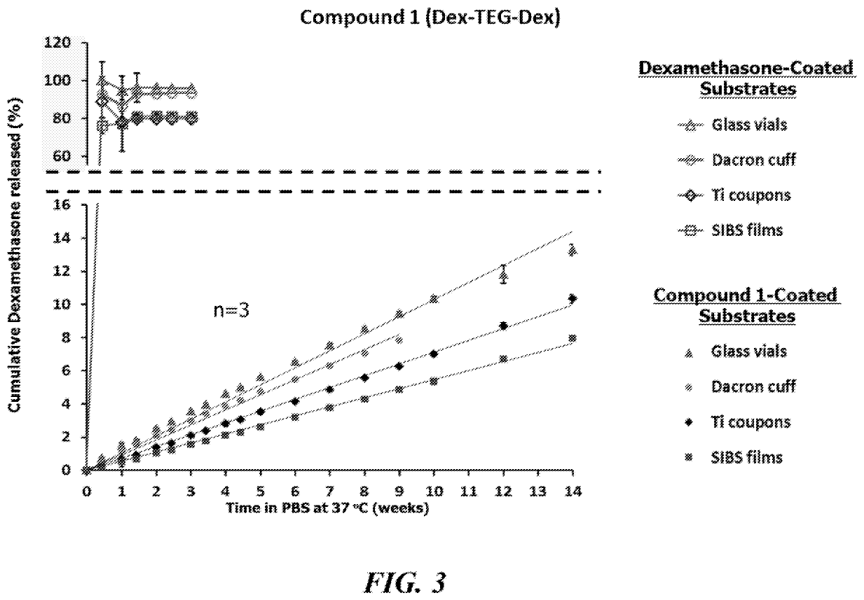

Different substrates including poly(styrene-block-isobutylene-block-styrene) (SIBS) films, titanium coupons, and fibrous Dacron (polyethylene terephthalate) meshes (FIG. 2A) were coated with compound 1 from acetone by drop coating (FIG. 2B) forming an amorphous (glassy) coating. Free dexamethasone was also drop coated onto the surface (FIG. 2C) and shows a crystalline coating morphology. The bottom of a glass vial was coated with compound 1 and dexamethasone from dichloromethane (DCM) using the drop coating method. Drug release from the coated surfaces in PBS at 37-C (FIG. 3) demonstrates a burst release for the crystalline dexamethasone coating and a slow, sustained drug release from the compound 1 coatings.

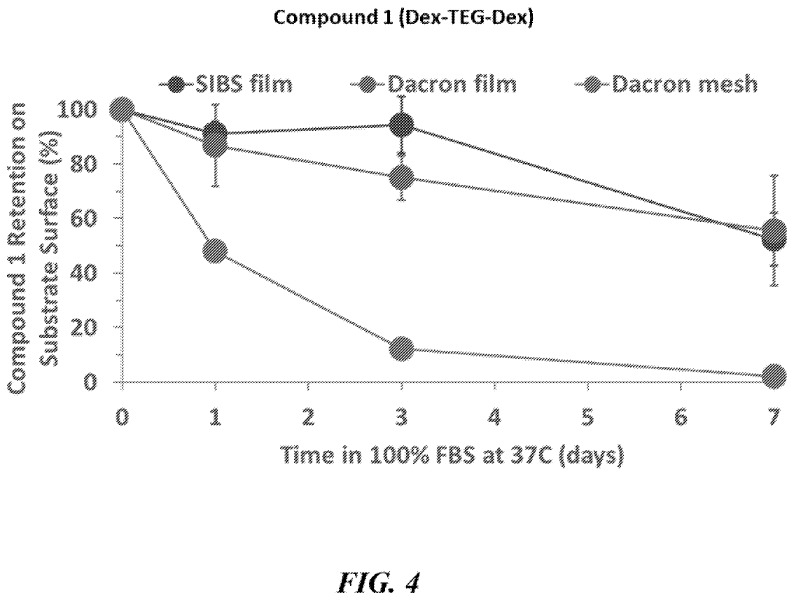

Compound 1 was drop coated with the same total amount of compound 1 from an organic solution (acetone) onto SIBS films, Dacron films, and fibrous Dacron meshes, and drug release was carried out in 100% FBS at 37 C. The coating was extracted from the substrates at different time points and was plotted as a percentage of coating retained over time (FIG. 4). The thin, high surface area coating on the fibrous Dacron mesh resulted in a quick release of coating from the surface. In contrast, the thicker, lower surface area coating on SIBS and Dacron films resulted in lower and longer sustained release from the surfaces.

### Example 2. Drop Coating Compound 1 on a Substrate

Compound 1 was dissolved in acetone at various concentrations (10 to 100 mg/mL) and was drop coated onto substrate surfaces of different sizes at various volumes (0.2 to 5 μL). One to five coating layers were applied to the substrates and the coatings were dried at ambient conditions overnight. Coatings were extracted and total Compound 1 loaded onto the surfaces was determined by HPLC.

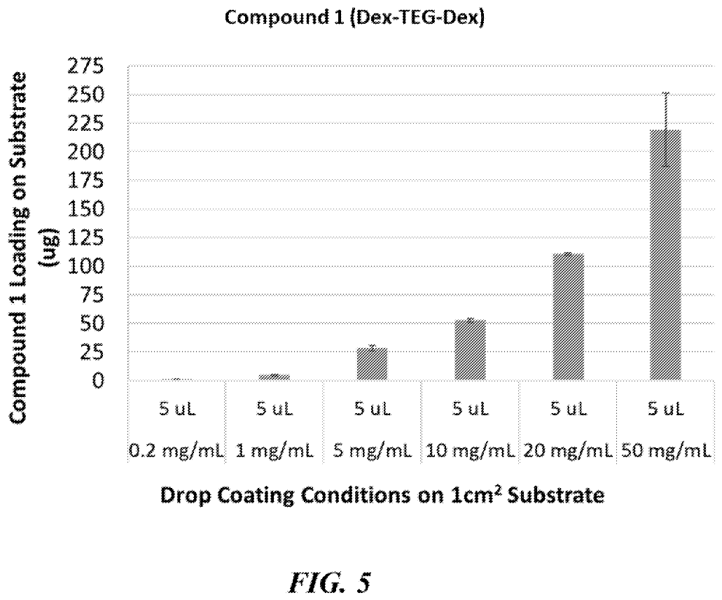

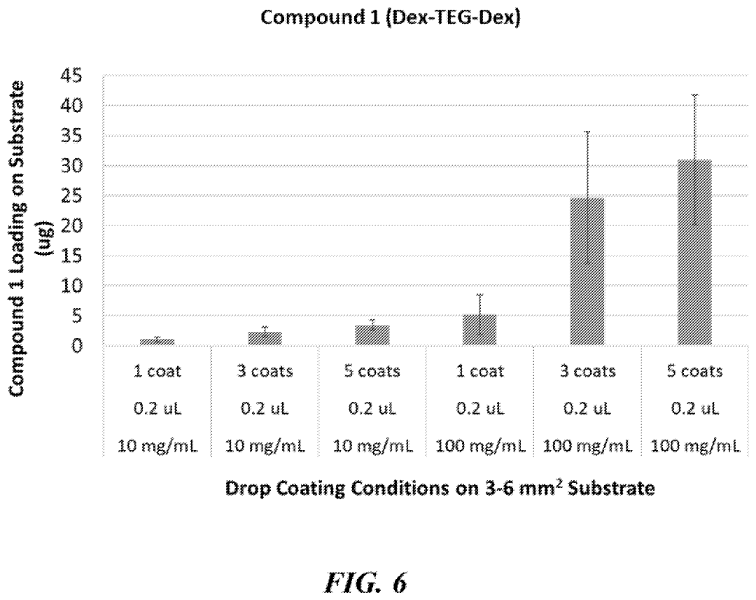

Drop coating led to predictable loading of Compound 1 on larger substrate surface areas (surface area of 1 cm²) (FIG. 5), but more variability was observed in the loading on smaller surface areas (surface area of 3-6 mm²) (FIG. 6). Coating solution on smaller substrates spread beyond the edge of the substrate and accounted for this observed variability. In cases where coating of only one side of the substrate is required, overflow of the coating beyond the edge of the substrate would be undesirable.

### Example 3. Dip Coating of Compound 1 on a Substrate

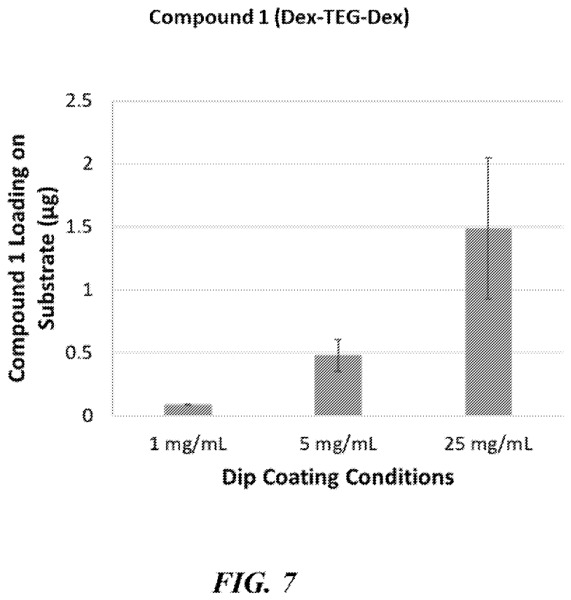

Compound 1 was dissolved in acetone at various concentrations (1 to 25 mg/mL). Substrate materials (surface area of 6-12 mm²) were dipped in the coating solution once for 5 seconds and then allowed to dry overnight in ambient conditions. Drug loading was determined by extracting the coating and determining the concentration by HPLC analysis (FIG. 7). Dip coating led to comparatively low drug loading compared to drop coating in example 5. This method resulted in coating of both sides of the substrate, which may be preferred in some applications while being undesirable in other applications.

### Example 4. Spray Coating of Compound 1 on a Substrate

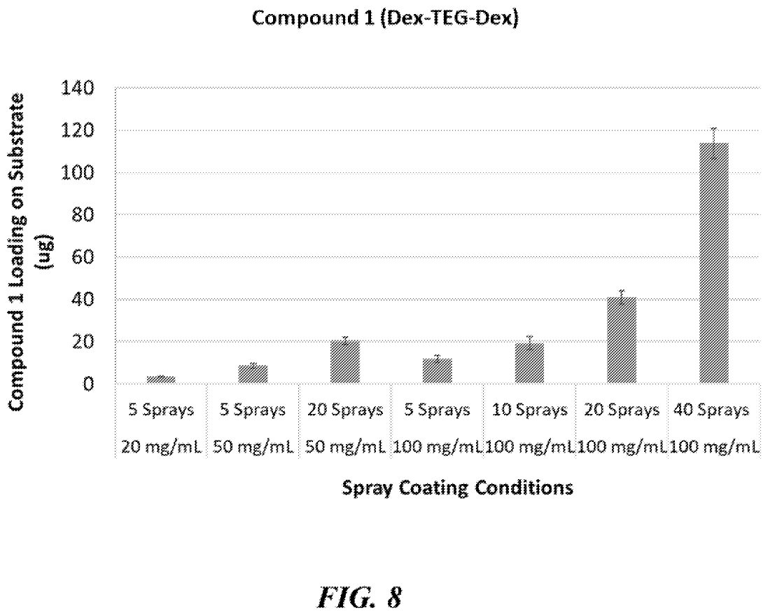

Compound 1 was dissolved in acetone at various concentrations (20 to 200 mg/mL). The coating solution of Compound 1 was loaded into a spray coater and sprayed onto substrate materials (surface area of 3-6 mm²) located below the spray nozzle (10 cm distance). Multiple sprays were dispensed with a delay ranging from 2 seconds to 1 minute between sprays. Following spraying, the coated substrates were dried overnight in ambient conditions. Drug loading was determined by extracting the coating and determining the concentration by HPLC analysis. Spray coating led to predictable drug loadings, related to the concentration of the spray solution and the number of sprays (FIG. 8). High drug loading, up to 120 μg, with the potential for higher loading with increased concentrations and/or number of sprays. Spray coating led to coating of only one side of the substrate.

### Example 5. Retention of Coating of Compound 1 During In Vitro Incubation

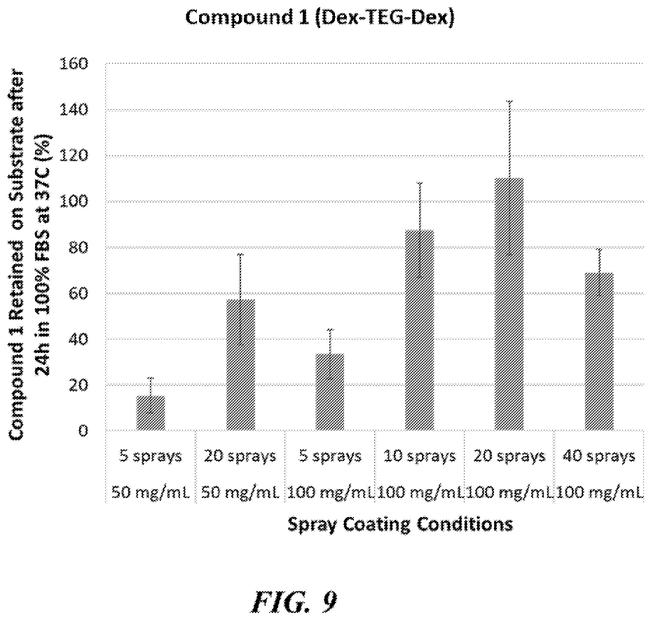

Substrates coated with Compound 1 by drop coating or spray coating were incubated in fetal bovine serum (FBS) to determine the amount of Compound 1 retained over time. Each coated substrate was placed in a glass vial containing 4 mL FBS and incubated at 37° C. on a rotating platform. After 1 to 4 days, the remaining coating was extracted and was determined by HPLC (FIG. 9). The amount of Compound 1 retained following 1-day incubation in FBS ranged from 15 to 100% of the initial amount loaded and roughly correlated with thickness (total loading) of the coating. Cracking of the thickest coating resulted in the lower retention than expected and may be improved by annealing.

### Example 6. Ethylene Oxide (ETO) Gas Sterilization of Coatings of Compound 1

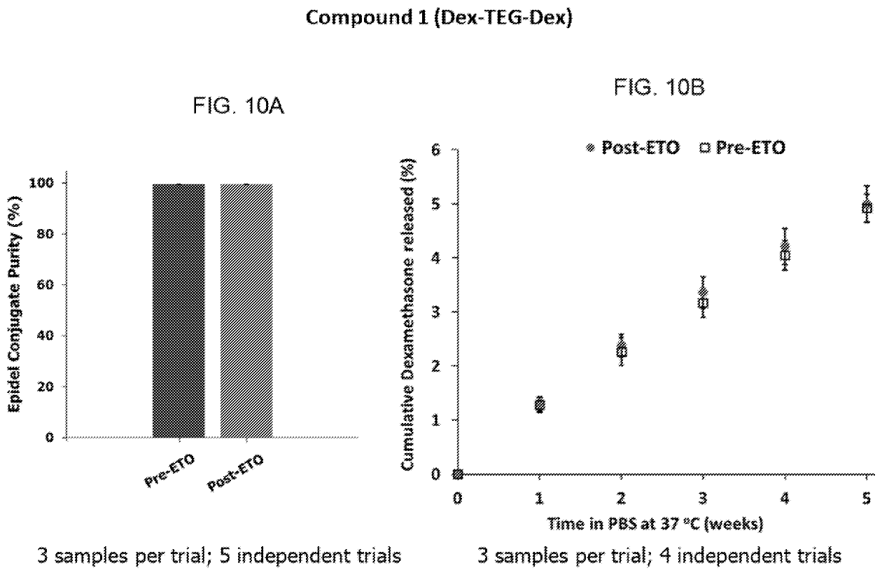

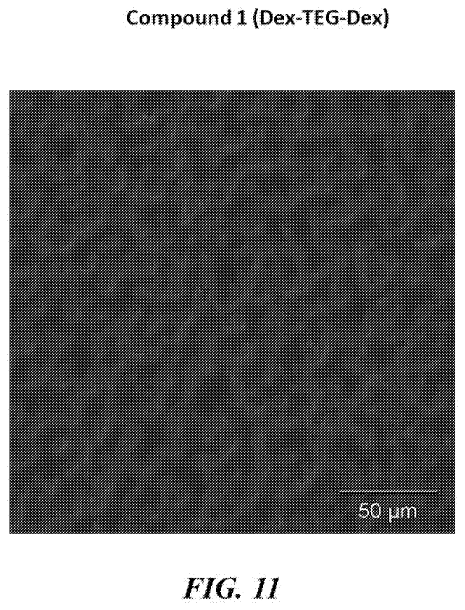

Compound 1 coated onto fibrous Dacron meshes were sterilized by ethylene oxide (ETO) gas at a temperature of 55° C. Pre- and post-ETO sterilized coated meshes were analyzed by HPLC to demonstrate no changes in coating (Compound 1) purity (FIG. 10A) and drug release (FIG. 10B) to demonstrate no changes in release properties due to the ETO sterilization process. Drug release was carried out in PBS at 37 C.

### Example 7: Compound 1 (Dex-TEG-Dex) Electrosprayed and Annealed to Form the Final Coating

Compound 1 was dissolved in acetone and was electrosprayed onto a polymer surface to form an intermediate glassy state coating. The sprayed surface was heated to ^{˜}150° C. to anneal compound 1 and obtain the final coating as shown in FIG. 11

### Example 8: Compound 1 (Dex-TEG-Dex) Coated onto an Angioplasty Balloon and Cardiac Stent

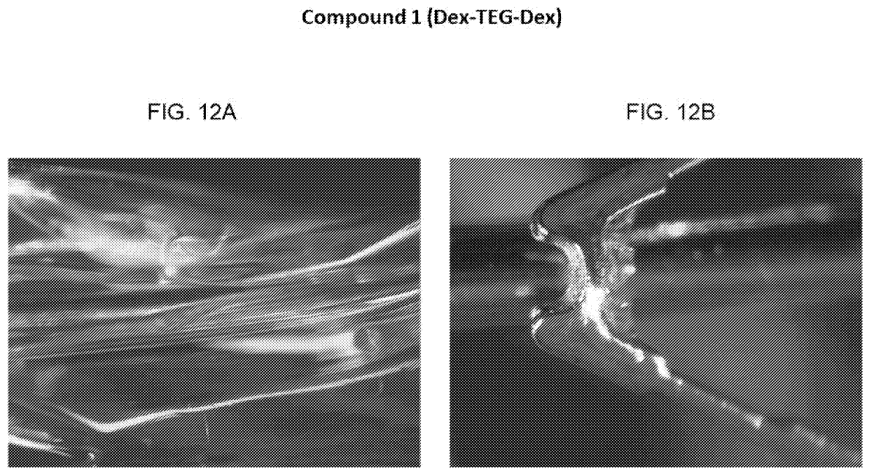

Compound 1 was dissolved in acetone and was drop coated onto an angioplasty balloon (FIG. 12A). Compound 1 was similarly spray coated onto a cardiac stent (FIG. 12B).

### Example 9. Rat Study with Coatings of Compound 1

Coatings of Compound 1 in various dexamethasone amounts (1.4-23.3 μg) were spray coated onto substrates as described in Example 4 and sterilized by ETO gas. Coated substrates were injected into the dorsal subcutaneous space of male SD rats with starting weights of 100-124 g (Envigo, Frederick, Md. USA). Animals were euthanized at 0, 3, 7 and 14 days and the skin surrounding the injection sites dissected. Coating remaining at each time point was quantified by HPLC.

Skin bleaching was noted for the skin surrounding the highest coating amount (23.3 μg) in day 14 animals. These results suggested that dexamethasone released from Compound 1 coatings were biologically active at doses relevant for local drug delivery.

### Example 10. Synthesis of Compound 2 (Hydrocortisone-Triethylene Glycol-Hydrocortisone), Coating onto Substrate Surface, and Drug Release from Coated Surface

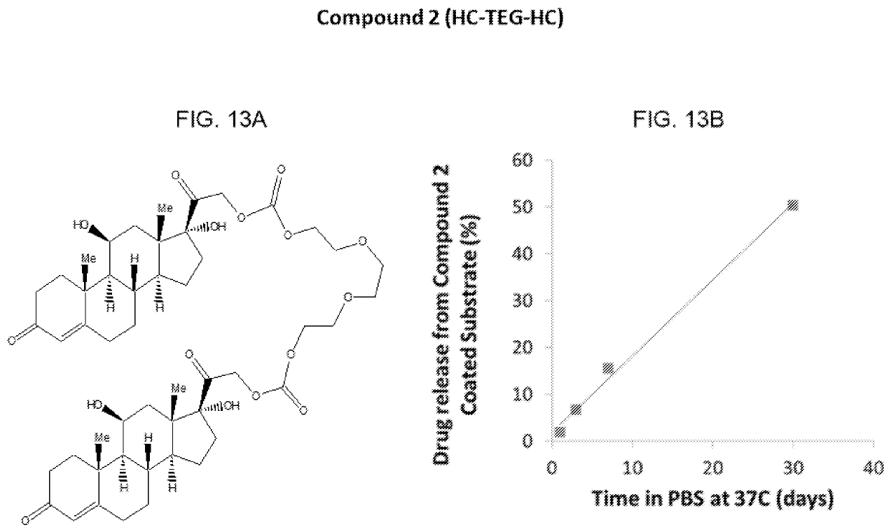

Hydrocortisone (42.68 g, 118 mmol) (USP grade) was recrystallized from hot anhydrous ethanol to give highly pure form of starting drug (16.94 g, 40% recovery). Recrystallized hydrocortisone (5 g, 13.8 mmol, 1 equiv) was suspended in dichloromethane (300 mL) on an ice bath (^{˜}0° C.) and triethylamine (3.86 mL, 2.80 g, 27.6 mmol, 2 equiv) and triethylene glycol bis(chloroformate) (1.70 mL, 2.28 g, 8.28 mmol, 0.6 equiv) were added to the mixture. The ice bath was allowed to warm to room temperature and the reaction was stirred overnight (^{˜}18 h). The solvent was removed in vacuo and the solid residue purified by normal phase automated column chromatography (hexane-ethyl acetate gradient). The product containing fractions were combined, concentrated in vacuo and the solid was crystallized from ethanol twice. The final product was dried under high vacuum at 50° C. for 4 h to give Compound 2 (FIG. 13A) as an off-white crystalline solid (1.92 g, 30%).

Compound 2: HPLC (mobile phase: H₂O/TFA and MeCN/TFA) 29.9 min; Elemental analysis: Anal. Calcd for C50H70O16: C, 64.78; H, 7.61; N, 0.00; Cl, 0.00 Found: C, 63.07; H, 7.71; N, <0.50; Cl <300 ppm. ¹H NMR (400 MHz, DMSO-d₆) δ (ppm) 0.78 (s, 6H, 2×C19 α-CH₃); 0.86 (dd, J=2.9 and 11.1 Hz, 2H, 2×C14-H), 0.99 (dd, J=4.0 and 13.7 Hz, 2H, 2×C8-H), 1.28 (t, J=6.0 Hz, 2H, 2×C7-CH); 1.36 (s, 6H, 2×C18-CH₃); 1.45 (m, 2H, 2×C7-H′); 1.65 (m, 6H, 2×C15-H, 2×C1-CH); 1.78 (dt, J=4.2 and 13.4 Hz, 2H, 2×C1-CH′), 1.92 (m, 6H, 2×C16-CH₂ and C6-CH₂), 2.09 (m, 2H, C6-CH₂′), 2.18 (m, 2H, 2×C9-CH₂), 2.42 (m, 6H, 2×C2-CH₂, 2×C9-CH); 3.56 (s, 4H, 2×TEG OCH₂); 3.64 (m, 4H, 2×TEG OCH₂); 4.22 (m, 4H, 2×TEG OCH₂); 4.26 (m, 2H, 2×C11-OCH); 4.36 (d, J=4.0 Hz, OH); 4.76 (d, 2H, AB, J=17.7 Hz, 2H, C21-CH₂O—); 5.11 (d, 2H, AB, J=17.7 Hz, 2H, C21-CH₂O′—); 5.42 (s, 2H, C17-OH); 5.56 (m, 2H, 2×alkene C4-CH). MS (ESI+) m/z: [M+H]+ Calcd for C₅₀H₇₁O₁₆ 927.47; Found 927.47.

Compound 2 was dissolved in DCM and was drop coated onto the bottom of a glass vial and solvent was evaporated to form the surface coating. Drug release was carried out in PBS at 37 C with buffer changes and was monitored by HPLC. Cumulative drug release calculated from the total drug in the coating (FIG. 13B) and demonstrates consistent drug release from the coated surface over time.

### Example 11. Synthesis of Compound 3 (Triamcinolone Acetonide-Triethylene Glycol-Triamcinolone Acetonide), Coating onto Substrate Surface, and Drug Release from Coated Surface

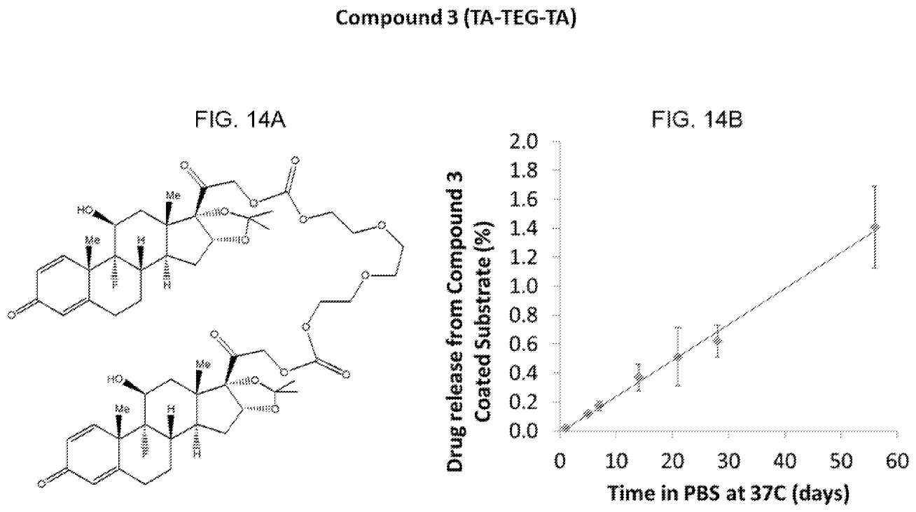

Triamcinolone acetonide (USP grade; 5 g, 11.5 mmol, 1 equiv) was suspended in dichloromethane (200 mL) on an ice bath (^{˜}0° C.) and triethylamine (3.21 mL, 2.33 g, 23.0 mmol, 2 equiv) and triethylene glycol bis(chloroformate) (1.42 mL, 1.90 g, 6.90 mmol, 0.6 equiv) were added to the mixture. The ice bath was allowed to warm to room temperature and the reaction was stirred overnight (^{˜}18 h). The solvent was removed in vacuo and the solid residue was purified by normal phase automated column chromatography (hexane-ethyl acetate gradient). The product containing fractions were combined, concentrated in vacuo and the solid was crystallized from acetonitrile twice. The final product was dried under high vacuum at 50° C. for 4 h to give Compound 3 (FIG. 14A) as an off-white crystalline solid (1.16 g, 19%).

Compound 3: HPLC (mobile phase: H₂O/TFA and MeCN/TFA) 36.2 min; Elemental analysis: Anal. Calcd for C₅₆H₇₂F₂O₁₈: C, 62.79; H, 6.78; N, 0.00; Cl, 0.00 Found: C, 62.64; H, 6.77; N, <0.50; Cl <225 ppm. ¹H NMR (400 MHz, DMSO-d₆) δ (ppm) 0.84 (s, 6H, 2×CH₃); 1.14 (s, 6H, 2×CH₃); 1.34 (s, 6H, 2×CH₃), 1.36 (m, 2H, 2×CH); 1.48 (s, 6H, 2×CH₃); 1.56 (m, 4H, 2×CH₂); 1.69 (d, J=7.0 Hz, 2H, 2×CH); 1.82 (m, 2H, 2×CH); 1.93 (dt, 2H, J=6.6 and 12.3 Hz, 2×CH); 2.03 (dt, 2H, J=3.2 and 13.3 Hz, 2×CH); 2.33 (dd, 2H, J=3.5 and 13.4 Hz, 2×CH); 2.44 (m, 2H, 2×CH); 2.53 (m, 2H, 2×CH); 2.62 (dt, 2H, J=6.0 and 13.3 Hz, 2×CH); 3.57 (s, 4H, 2×TEG OCH₂); 3.65 (m, 4H, 2×TEG OCH₂); 4.20 (m, 2H, 2×OCH), 4.24 (m, 4H, 2×TEG OCH₂); 4.75 (d, 2H, AB, J=18.0 Hz, 2H, C21-CH₂O—); 4.87 (d, 2H, J=4.6 Hz, 2×C11-OCH), 5.18 (d, 2H, AB, J=18.0 Hz, 2H, C21-CH2O—); 5.48 (d, 2H, J=3.9 Hz, 2×C11-OH); 6.02 (m, 2H, 2×alkene C4-CH); 6.23 (dd, 2H, J=10.1 and 1.9 Hz, CH, 2×alkene _{C2}—CH); 7.29 (d, 2H, C1-CH 2×alkene CH, 10.1 Hz, 2H). MS (ESI+) m/z: [M+H]+ Calcd for C₅₆H₇₃O₁₈F₂ 1071.48; Found 1071.48.

Compound 3 was dissolved in DCM and was drop coated onto the bottom of a glass vial and solvent was evaporated to form the surface coating. Drug release was carried out in PBS at 37 C with buffer changes and was monitored by HPLC. Cumulative drug release calculated from the total drug in the coating (FIG. 14B) and demonstrates consistent drug release from the coated surface over time.

### Example 12. Synthesis of Compound 4 (Dexamethasone-Hexane-Dexamethasone), Coating onto Substrate Surface, and Drug Release from Coated Surface

Dexamethasone (157 mg, 0.40 mmol, 1.0 equiv) was dissolved in THF (20 mL) under nitrogen and phosgene solution (2.86 mL of a 1.4 M solution in toluene, 4.0 mmol, 10 equiv) was added to the solution. The mixture was stirred at room temperature for 6 h. The excess phosgene and solvents were removed in vacuo and the solid residue was redissolved in DCM (20 mL). 1,6-hexanediol (24 mg, 0.20 mmol, 0.5 equiv) was added to the solution with pyridine (65 μL, 63 mg, 0.80 mmol, 2.0 equiv). The mixture was stirred overnight at room temperature (^{˜}18 h). The mixture was concentrated onto reverse phase silica (1 g) and purified by automated reverse phase chromatography (acetonitrile-water). The product containing fractions were combined and concentrated in vacuo. The final product was dried under high vacuum at 50° C. for 4 h to give Compound 4 as an off-white glassy solid (38 mg, 20%).

Compound 4: HPLC (mobile phase: H₂O/TFA and MeCN/TFA) 36.1 min; ¹H NMR (400 MHz, DMSO-d₆) δ (ppm) 0.79 (d, J=7 Hz, 6H, 2×C16 α-CH₃); 0.90 (s, 6H, Dex 2×C18-CH₃); 1.08 (m, 2H, 2×C16-H); 1.37 (m, 6H, 2×CH₂, 2×CH); 1.49 (s, 6H, C19-CH₃); 1.63 (m, 8H, 4×CH₂); 1.78 (m, 2H, C15-CH₂); 2.15 (m, 4H, 2×CH₂); 2.40 (m, 4H, 2×CH₂); 2.62 (m, 2H, 2×CH); 2.88 (m, 2H, 2×C12-CH); 4.13 (m, 6H, 2×OCH₂, 2×C11-OCH); 4.77 (d, 2H, AB, J=18.5 Hz, 2H, C21-CH₂O—); 5.07 (d, 2H, AB, J=18.5 Hz, 2H, C21-CH₂O—); 5.20 (s, 2H, C17-OH); 5.42 (s, 2H, C17-OH); 6.01 (d, 2H, J=1.9 Hz, alkene C4-CH); 6.22 (dd, 2H, J=10.1 and 1.9 Hz, CH, alkene C2-CH); 7.30 (d, 2H, C1-CH alkene CH, J=10.1 Hz, 2H). MS (ESI+) m/z: [M+H]+ Calcd for C₅₂H₆₉F₂O₁₄ 955.47; Found 955.47.

Compound 4 was spray coated on substrates (surface area of 3-6 mm²) in a similar manner to compound 1 as shown in Example 4. Using 20 sprays of a 20 mg/mL coating solution, the loading of dexamethasone in the form of Compound 4 was 3.1±1.0 μg.

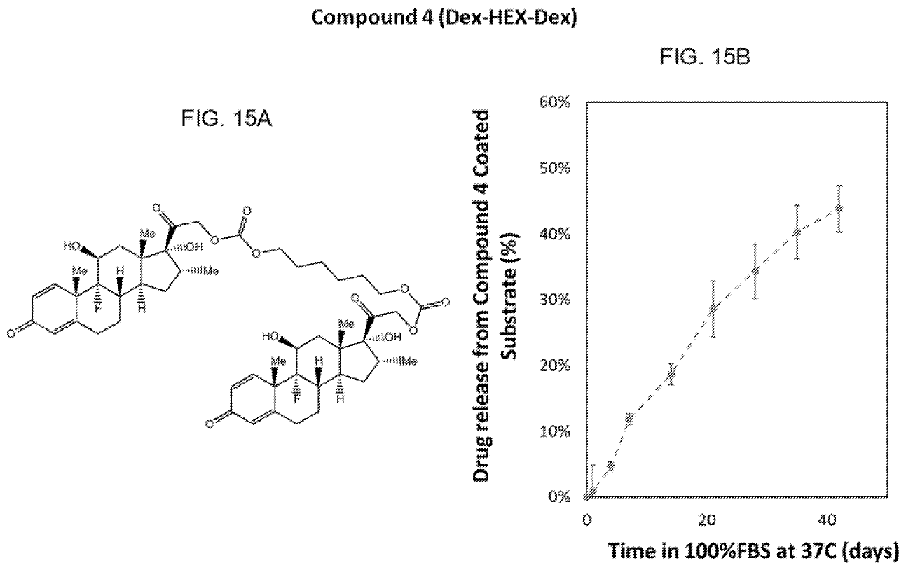

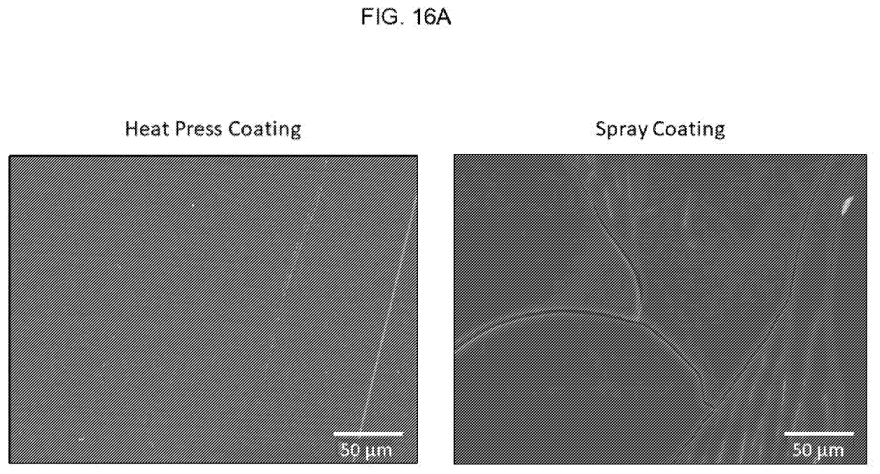

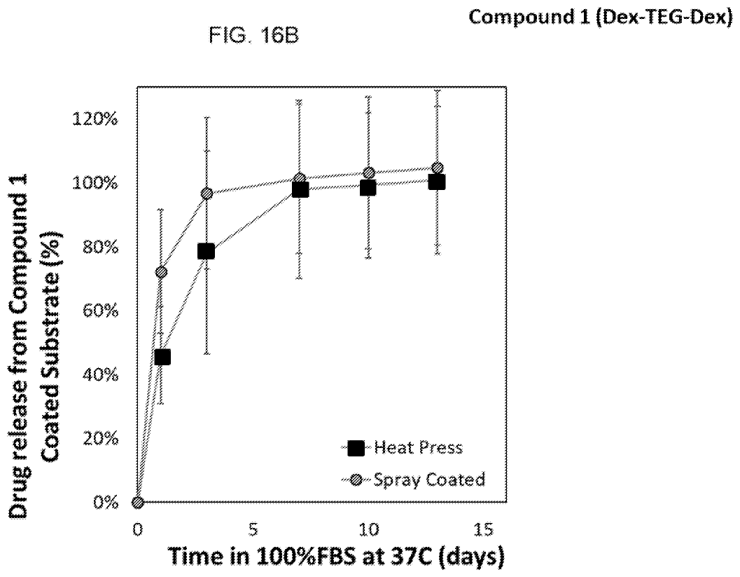

To study the drug release from coated surfaces, Compound 4 was drop coated on a substrate (surface area of 7 mm²) and solvent was evaporated to form the surface coating. Drug release was carried out in 100% FBS and agitation at 37 C with buffer changes and was monitored by HPLC. Cumulative drug release calculated from the total drug in the coating (FIG. 15B) and demonstrates consistent drug release from the coated surface over time.

### Example 13. Heat Press Process for Coating a Substrate Surface and Drug Release from Coated Surface

A crystalline powder of compound 1 (Dex-TEG-DEX) was distributed evenly across the surface to be coated and a sheet of aluminum foil, or another material, was placed on top. The powder was pre-melted without additional pressure at 185° C. for 2 minutes. The dexamethasone sample was then compressed at 1500 PSI for 30 seconds.

Using this heat press process yielded a surface coating with a larger thickness and therefore a higher drug load (37.1±8.1 μg/mm²) in comparison to spray-coating and drying (7.8±1.2 μg/mm²) with less cracking and surface defects (FIG. 16A). Spray-coated surface coatings were produced as described in Example 4 using a 50 mg/mL solution and 80 sprays. The heat press method shows reduced cracking of the coating at 4 to 5× the thickness leading to higher defect-free drug loading. At similar drug densities, heat press and spray coating have similar drug release profiles.

Depending on Compound 1 mass, a wide range of drug loadings (4-37 ag/mm²) was achieved by applying temperatures from 150 C-180 C and pressures from 700-3000 PSI for 30 s-10 min. Temperatures and pressures were applied using a Carver laboratory press with metallic heated plates and the coating was deposited on polymer and metallic substrates.

Drug release profiles from Compound 1 surface coatings with similar drug densities (8.2-8.3 μg/mm²) formed from the heat press process and spray coating process were similar (FIG. 16B), confirming that drug release is a material property and not influenced by coating technique.

### Example 14. Heat Press Process for Coating a Substrate Surface with Similar Drug Density at Different Surface Areas

A crystalline powder of Compound 1 was distributed evenly across a sheet of aluminum foil and pre-melted without applied pressure at 185° C. for 2 minutes to convert to an amorphous state. Pre-melted Compound 1 was ground manually using a mortar and pestle.

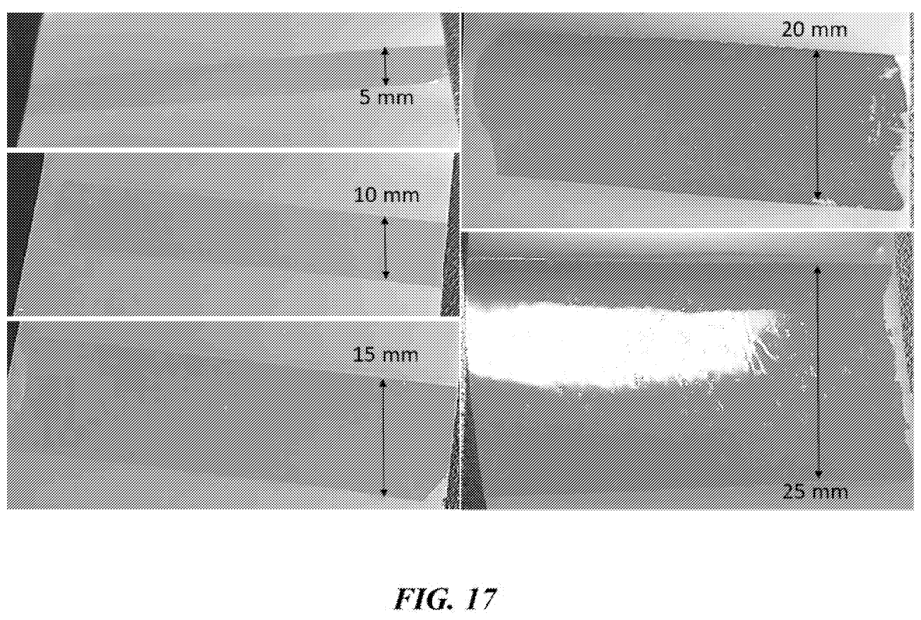

Polyester strips were used as a coating template and were placed at various distances apart (5 mm to 25 mm) on top of a substrate. Amorphous Compound 1 was distributed in small piles on the exposed substrate (between the polyester strips), covered with aluminum foil, and transferred to a Carver Laboratory press with heated metallic plates at a temperature of 180° C. A pressure of 1200 PSI was applied to the substrate for 1 minute to coat the exposed substrate. After initial coating, the substrate was removed from the heated press and allowed to cool gradually at ambient conditions. Once cooled, the aluminum foil was removed from the top and the coating was annealed at 180° C. for 20 seconds. Coatings looked uniform and free of defects (FIG. 17).

### Example 15. Rat Study of Compound 1 Coated on Different Substrates

Compound 1 was dissolved in acetone and spray coated onto thin films of a polymeric substrate or drop coated onto discs of a different polymeric substrate. Both coatings were dried at ambient conditions over night and sterilized using Ethylene oxide (ETO) gas. Coated substrates were then implanted into the dorsal subcutaneous space in female Wistar rats with starting weights of 250-290 g. Animals were euthanized at the peak of inflammatory phase in days 3 and 7. The cells in the surrounding area of both substrates were assessed by morphometric image analysis on cross-section of excised substrates with associated tissue that were stained for CD68 (inflammatory cell marker), ASMA (fibroblast marker) and DAPI (nuclease marker). The number of recruited CD68 positive cells were reduced in response to both substrates coated with compound 1 compared to non-coated substrates. Thus, compound 1 suppresses inflammation in device related foreign body reaction and fibrosis in the rat subcutaneous model regardless of coating method or substrate.

### Example 16. Rat Study with Coatings of Compound 1, Compound 4 and Dexamethasone to Investigate Suppression of Fibrosis

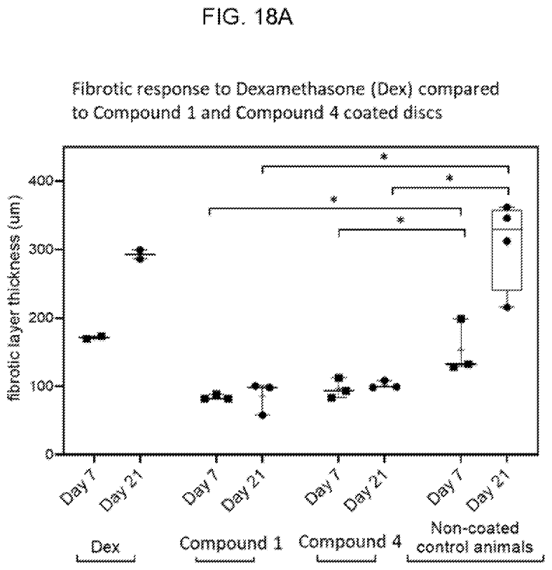

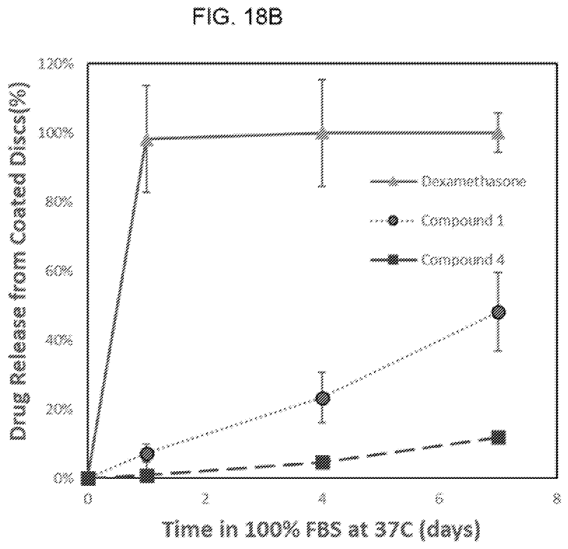

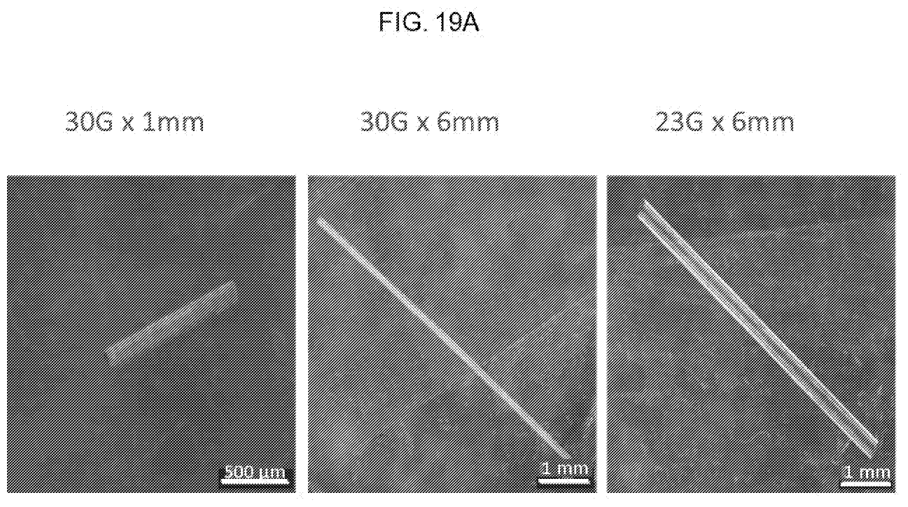

Compound 1 and Compound 4 were dissolved in acetone and drop coated onto polymeric discs (diameter: 3 mm, thickness: 2 mm). Discs were also drop coated using dexamethasone dissolved in acetone to obtain similar drug amounts as discs coated with compounds 1 and 4. The coatings were dried at ambient conditions over night and sterilized using Ethylene oxide (ETO) gas. Coated substrates were then implanted into the dorsal subcutaneous space in female Wistar rats with starting weights of 250-290 g. Animals were euthanized at day 7 (inflammatory phase) and day 21 (fibrotic phase) to assess the tissue response to coated versus non-coated implants. The thickness of the collagenous capsule wall surrounding the implant was quantified by morphometric image analysis on cross-section of excised capsules with associated tissue that were stained with Masson's Trichrome. The capsule wall diameter (fibrotic layer thickness) around non-coated implants increased by two folds from day 7 to day 21. The tissue surrounding the Dexamethasone coated implants did not show any difference to non-coated discs while Compounds 1 and 4 were statistically thinner at both time points. Thus, dexamethasone is not effective in suppressing device related fibrosis compared to Compounds 1 and 4 (FIG. 18A). This correlates well with in vitro drug release profiles demonstrating a burst release for dexamethasone and sustained release for Compounds 1 and 4 (FIG. 18B).

### Example 19. Compound 1 (Dexamethasone-Triethylene Glycol-Dexamethasone; Dex-TEG-Dex) Extruded into Glassy State Injectable Cylinders or a Glassy State Cylindrical Drug Depot, and Release Drug Via Surface Erosion

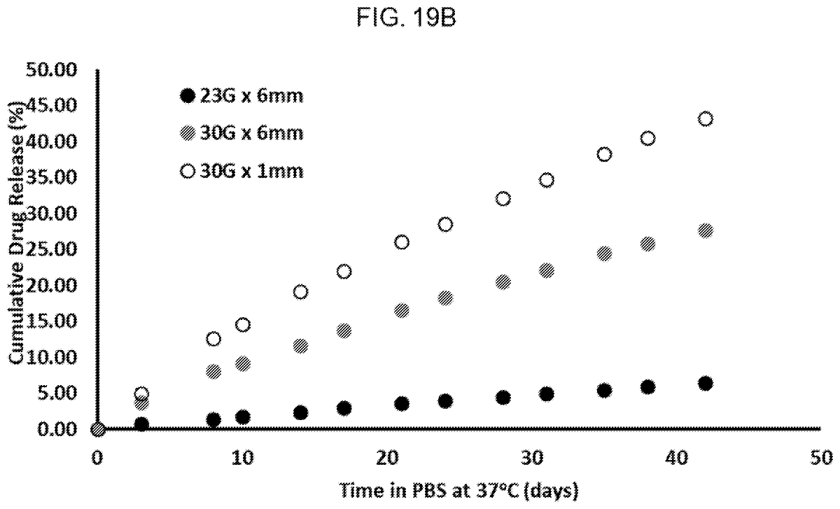

Compound 1 was formed into cylinders by heat extrusion from the intermediate glassy state by pre-melting compound 1 prior to extrusion. The cylinders were extruded with 23G and 30G nozzles and cut to different lengths as shown in FIG. 19A. In vitro drug release was carried out in PBS at 37 C and drug release was quantified by HPLC. Cumulative drug release was plotted as a percentage of total drug in each cylinder as shown in FIG. 19B and drug release occurred via surface erosion. The cylinders can be used as a drug depot within a medical device or injected adjacent to the medical device to release dexamethasone.

### Example 20. Compound 1 (Dex-TEG-Dex) can Formed into Fibrous Mesh Drug Depot in the Glassy State

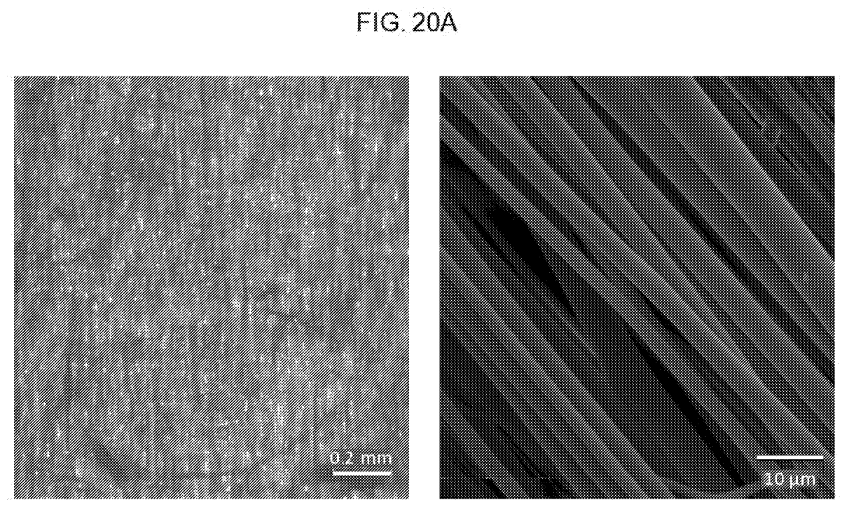

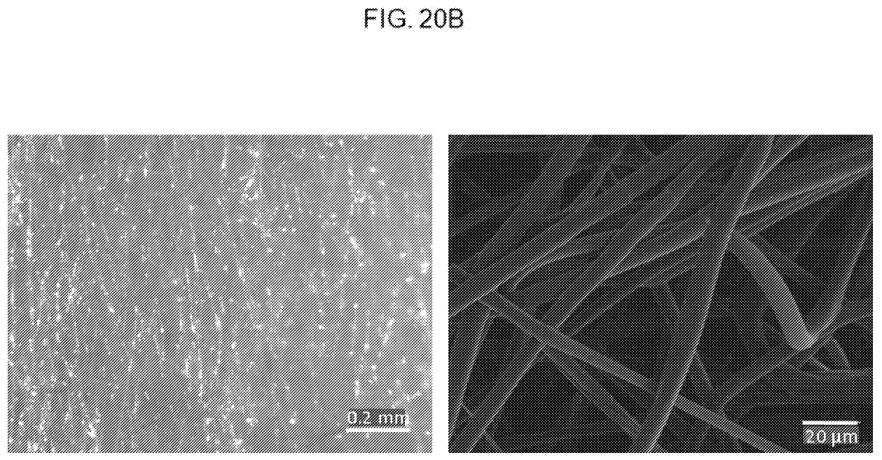

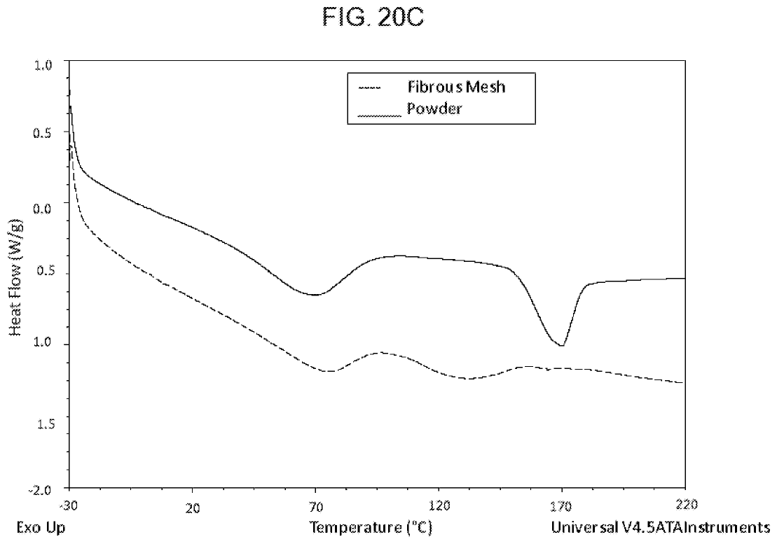

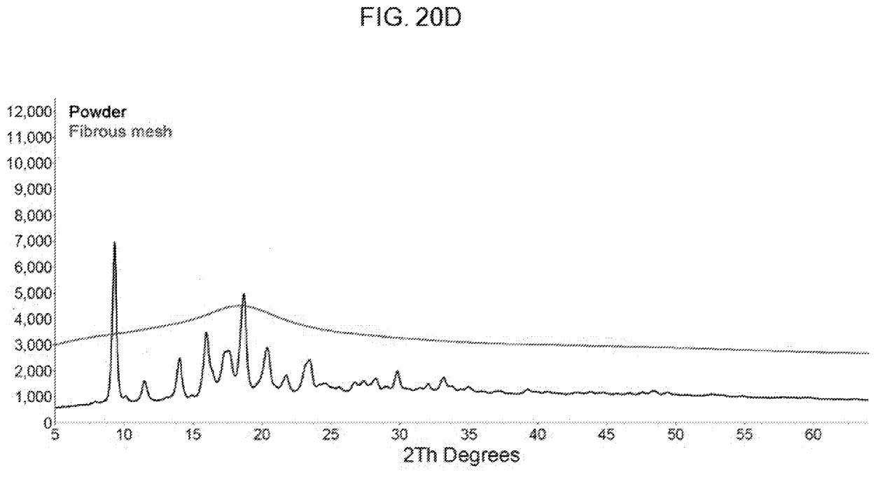

Non-woven fibrous meshes with aligned (FIG. 20A) and unaligned (FIG. 20B) morphologies were prepared by electrospinning. Compound 1 was dissolved in tetrahydrofuran (THF) and was electrosprayed onto a cylindrical rotating mandrel to obtain aligned fibers or onto a stationary collector surface to obtain unaligned fibers. Compound 1 as the starting powder and solvent-processed fibrous mesh were tested by DSC (FIG. 20C) and PXRD (FIG. 20D) to confirm the meshes were in the glassy state. The fibrous meshes can be inserted as a wafer adjacent to a medical device or can be electrospun around the device or a device component forming a fibrous pouch that acts as a drug depot to release dexamethasone.

### Example 21. Injectable Cylinders of Compound 1 (Dex-TEG-Dex) can be Injected Adjacent to a Medical Device Such as a Suture to Release an Anti-Inflammatory Corticosteroid

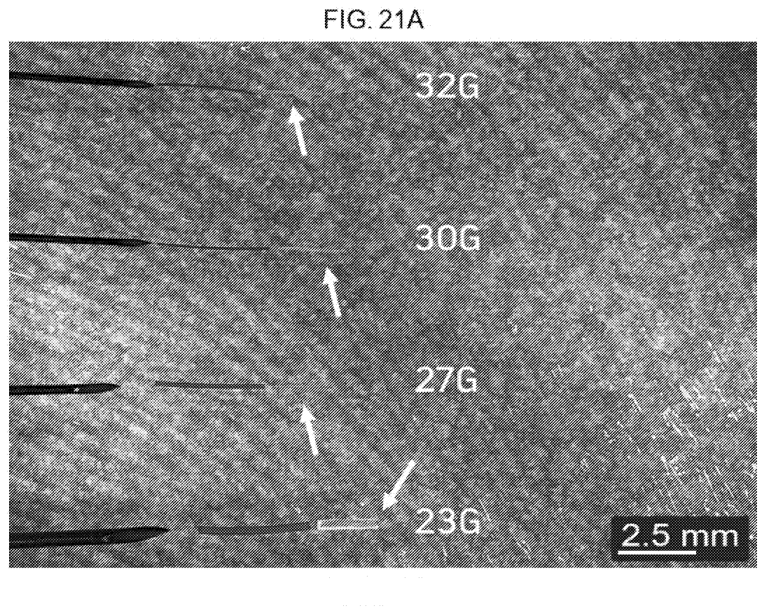

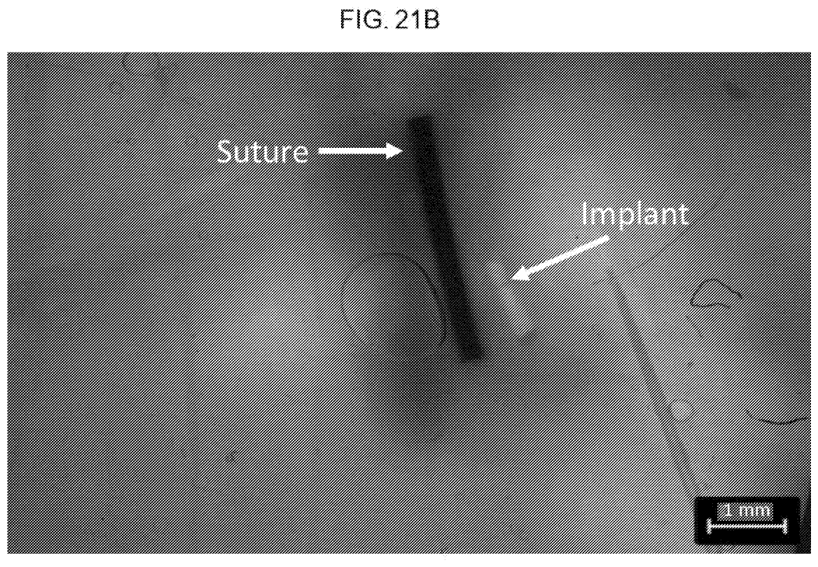

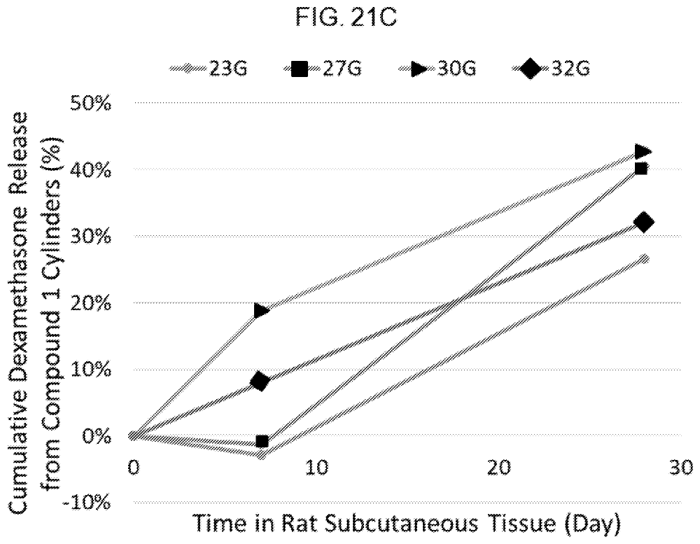

Cylinders of Compound 1 in various diameters (23G-32G) and 2 mm in length were processed as described in Example 1 and were loaded into needles with a piece of medical sutures as shown in FIG. 21A. The loaded needles were sterilized by ethylene oxide gas. The cylinders and sutures were injected into the dorsal subcutaneous space of male Sprague Dawley rats with starting weights of 100-124 g (Envigo, Frederick, Md. USA). Animals were euthanized at 7 and 28 days and the skin surrounding the injection sites were dissected. An image of a cylinder and suture in the subcutaneous tissue is shown in FIG. 21B. Pellet drug content at each time point was calculated from in-situ image analysis, confirmed by HPLC, and was plotted as cumulative drug release from the cylinders (FIG. 21C). The results show sustained dexamethasone release from injectable compound 1 cylinders in the subcutaneous tissue of rats.

What is claimed is:

1. A system comprising an article body and a steroid material, the steroid material comprising a compound of formula (A-VIII):
D1-L-D2 (A-VIII)
or a pharmaceutically acceptable salt thereof,
wherein
(i) each of D1 and D2 is, independently, a steroid radical; and L is a linker covalently linking D1 to D2, and
(ii) the steroid material comprises the compound of formula (A-VIII) in an amount of at least 90% (w/w).

2. An article comprising an article body and a steroid material, the steroid material comprising a compound of formula (A-VIII):
D1-L-D2 (A-VIII)
or a pharmaceutically acceptable salt thereof,
wherein
(i) each of D1 and D2 is, independently, a steroid radical; and L is a linker covalently linking D1 to D2, and
(ii) the steroid material comprises the compound of formula (A-VIII) in an amount of at least 90% (w/w).

3. The system or article of either one of claims 1 or 2, wherein the steroid material is in the form of a second body, the second body being packaged with (e.g., as a kit) or affixed to the article body.

4. The system or article of claim 3, wherein the second body is affixed to the article body with an adhesive, a clamp, or a bolt.

5. The system or article of either one of claims 1 or 2, wherein the steroid material is in the form of a coating, the coating being on (e.g., at least partially covering) at least one surface of the article body.

6. The system or article of any one of the preceding claims, wherein the article body is an implant (e.g., sensor implant).

7. The system or article of any one of the preceding claims, wherein the steroid material (or second body or coating) is free of (e.g., comprises less than 5 wt. %, less than 2 wt. %, less than 1 wt. %) a controlled release excipient.

8. The system or article of any one of the preceding claims, wherein the steroid material provides release of free steroid therefrom without the need of a controlled release excipient.

9. The system or article of any one of the preceding claims, wherein the steroid material (or article body or coating) releases D1 and D2 at 37° C. in 100% bovine serum or at 37° C. in PBS at a rate such that t₁₀ is greater than or equal to 1/10 of t₅₀.

10. The system or article of any one of the preceding claims, wherein the steroid material (or article body or coating) comprises from 0.01 to 10% (w/w) of one or more plasticizing agents.

11. The system or article of any one of the preceding claims, wherein the steroid material is a surface coating or a co-implant (e.g., a drug depot co-implanted with the article body).

12. The system or article of claim 11, wherein the surface coating coats at most half (e.g., less than one-quarter, less than one-eighth, or less than one sixteenth) of the article body.

13. The system or article of claim 11, wherein the surface coating is a continuous layer on the article body (e.g., the surface coating does not contain cracks, fissures, gaps, or the like).

14. The system or article of claim 11, wherein the co-implant (e.g., the drug depot co-implanted with the article body) is selected from a pellet, a cylinder, a hollow tube, a microparticle, a nanoparticle, or a shaped article.

15. The system or article of claim 11, wherein the co-implant (e.g., the drug depot co-implanted with the article body) is separate from the article body (e.g., an implantable medical device).

16. The system or article of claim 11, wherein the co-implant (e.g., the drug depot co-implanted with the article body) is affixed (e.g., adhesively affixed, screwed, bolted, or the like) to the article body (e.g., an implantable medical device).

17. The system or article of any one of the preceding claims, wherein D1 and D2 are each anti-inflammatory steroids (e.g., dexamethasone), or pharmaceutically acceptable salts thereof, in their free form.

18. The system or article of any one of the preceding claims, wherein D1 and D2 are each intraocular pressure (IOP) lowering steroids (e.g., anecortave), or pharmaceutically acceptable salts thereof, in their free form.

19. A method of providing an implant into an individual, the method comprising (i) implanting an implant article into the individual at an implant location, and (ii) implanting a steroid material into the individual, the steroid material being implanted in proximity (e.g., within 20 mm, within 10 mm, within 5 mm, within 3 mm, or less) to the implant location, the steroid material being as described in an one of the preceding claims.

20. The method of claim 19, wherein the implant article and the steroid material are administered concurrently (e.g., wherein the steroid material is affixed to or coated on the implant article).

21. The method of claim 19, wherein the implant article and the steroid material are administered sequentially (e.g., the implant article or the steroid material implanted first, followed by implant of the other).

22. The method of any one of the preceding claims, wherein the steroid material remains implanted in the individual for at least 1 day, 1 week, 2 weeks, 1 month, or longer.

23. The method of any one of the preceding claims, wherein at least a portion of the steroid material is uptaken by the individual at a rate sufficient to produce a physiological effect (e.g., reduce inflammation (e.g., minimize an inflammatory response), reduce pressure (e.g., lower intraocular pressure (IOP)), or the like) in or around the implant location.

24. The method of any one of the preceding claims, wherein the rate at 37° C. in 100% bovine serum or at 37° C. in PBS is such that t₁₀ is greater than or equal to 1/10 of t₅₀.

25. The method of any one of the preceding claims, wherein inflammation in or around the implant location is reduced (e.g., by at least 10%, by at least 20%, by at least 30%, by at least 50%).

26. The method of any one of the preceding claims, wherein inflammation in or around the implant location is measured by fibrotic layer thickness (e.g., μm), collagen content (e.g., μM), hydroxyproline content (e.g., μM), inflammatory cell count (e.g. number of macrophages, foreign body giant cells, etc.), inflammatory cell type (e.g. myofibroblasts), inflammatory cytokines (e.g. IL-1β, TNF-α, etc.), or the like.
